(19) United States
(12) Patent Application Publication
(10) Pub. No.: US 2024/0336642 A1
(43) Pub. Date: Oct. 10, 2024

(54) INHIBITORS OF 12/15-LIPOXYGENASE

(71) Applicants: The General Hospital Corporation, Boston, MA (US); The Regents of the University of California, Oakland, CA (US)

(72) Inventors: Klaus van Leyen, Medford, MA (US); Yi Zheng, Cambridge, MA (US); Theodore Holman, Santa Cruz, CA (US); Graham Johnson, Sanbornton, NH (US); Robert Jason Herr, Voorheesville, NY (US)

(21) Appl. No.: 18/294,441

(22) PCT Filed: Aug. 5, 2022
(86) PCT No.: PCT/US2022/074621

§ 371 Date: Feb. 1, 2024

Related U.S. Application Data
(60) Provisional application No. 63/231,061, filed on Aug. 9, 2021.

(51) Int. Cl. C07F 9/653; A61K 31/421; A61K 31/422; A61K 31/4439; A61K 31/454; A61K 31/501; A61K 31/506; A61K 31/5377; A61K 31/675; A61P 9/10; A61P 25/28; C07D 263/48; C07D 413/12

(52) U.S. Cl. CPC C07F 9/653; A61K 31/421; A61K 31/422; A61K 31/4439; A61K 31/454; A61K 31/501; A61K 31/506; A61K 31/5377; A61K 31/675; A61P 9/10; A61P 25/28; C07D 263/48; C07D 413/12

(57) ABSTRACT

The present application provides oxazoles-containing compounds, or a pharmaceutically acceptable salt thereof, that are 12/15-LOX inhibitors. Pharmaceutical compositions and methods of using these compounds for treating various conditions wherein 12/15-LOX is implicated (such as stroke) are also provided.

### CLAIM OF PRIORITY

This application claims priority to U.S. Patent Application Ser. No. 63/231,061, filed on Aug. 9, 2021, the entire contents of which are hereby incorporated by reference.

### FEDERALLY SPONSORED RESEARCH OR DEVELOPMENT

This invention was made with government support under grant No. NS106854 awarded by National Institutes of Health. The government has certain rights in the invention.

### TECHNICAL FIELD

This invention relates to 2-(2,3,5-trisubstituted phenyl) oxazole compounds, useful in treating or preventing diseases and conditions in which 12/15-lipoxygenase (“12/15-LOX”)—or the ALOX15 gene which encodes it—is implicated.

### BACKGROUND

There are numerous deadly diseases affecting current human population. For example, stroke is a leading cause of mortality and disability worldwide and the economic costs of treatment and post-stroke care are substantial. Every year, more that 14 million people are affected by stroke, and over 6 million stroke patients die from this condition and associated complications.

### SUMMARY

This disclosure is based, at least in part, on a realization that 2-(2,3,5-trisubstituted phenyl) oxazole compounds potently inhibit 12/15-LOX. Hence, the compounds of this disclosure are advantageously useful to treat or prevent various disorders where 12/15-LOX is implicated in the pathology of the disorder (e.g., stroke).

In one general aspect, the present disclosure provides a compound of Formula (I):

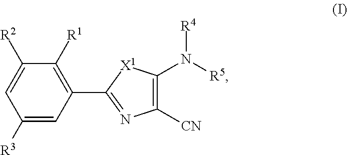

or a pharmaceutically acceptable salt thereof, wherein R¹, R², R³, R⁴, R⁵, and X¹ are as described herein.

In another general aspect, the present disclosure provides a pharmaceutical composition comprising a compound of Formula (I), or a pharmaceutically acceptable salt thereof, and a pharmaceutically acceptable carrier.

In yet another general aspect, the present disclosure provides a method of treating or preventing a disease or disorder in which 12/15-lipoxygenase (12/15-LOX) is implicated in the pathology (e.g., any one of the disorders described herein), the method comprising administering to a subject in need thereof a therapeutically effective amount of any one of the compounds described herein, or a pharmaceutically acceptable salt thereof, or a pharmaceutical composition comprising same.

Unless otherwise defined, all technical and scientific terms used herein have the same meaning as commonly understood by one of ordinary skill in the art to which the present application belongs. Methods and materials are described herein for use in the present application; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the present application will be apparent from the following detailed description and figures, and from the claims.

### DESCRIPTION OF DRAWINGS

FIG. 1 contains chemical schemes showing in vivo conversion of prodrug compounds 51, 52, 53 to their parent compounds 22, 1, and 1, respectively.

FIG. 2 contains bar graphs showing that compound 1 provided significant protection in both male and female mice, reducing infarct sizes at 24 h by 33.4% and 37.2%, respectively when given intraperitoneally.

FIG. 3 contains bar graphs showing that deuterated compound 44 has similar efficacy compared to compound 1.

FIG. 4 contains bar graphs showing that compound 32 leads to 31.8% infarct size reduction.

FIG. 5 contains bar graphs showing efficacy of compound 52 after intravenous delivery. Compound 52 was dissolved in in 50% Captisol and infused intravenously at a dosage of 40 mg/kg, immediately after reperfusion. 31.7% infarct size reduction at 24 h, p<0.01.

FIG. 6 contains bar graphs showing efficacy of compound 1 after intravenous delivery. Compound 1 was dissolved in 10% Solutol/90% PEG400 and infused intravenously at a dosage of 40 mg/kg, 2 h after reperfusion. 34.1% infarct size reduction at 24 h. n=8/group, p<0.01.

FIG. 7 contains bar graph showing infarct size reduction in mice after administration of cmpd. 1 at 20 mg/kg i.v. 2 hours post reperfusion.

FIG. 8A contains line plot showing Garcia score for mice after administration of cmpd. 1 40 mg/kg 2 hours post reperfusion. At 4 weeks after stroke, 68.5% improvement compared to vehicle treatment (p<0.01).

FIG. 8B contains line plot showing Corner test results for mice after administration of cmpd. 1 40 mg/kg 2 hours post reperfusion. At 4 weeks after stroke, 75.4% improvement compared to vehicle treatment (p<0.0001).

FIG. 8C contains line plot showing foot fault test results for mice after administration of cmpd. 1 40 mg/kg 2 hours post reperfusion. At 4 weeks after stroke, 77.0% improvement compared to vehicle treatment (p<0.0001).

FIG. 8D contains line plot showing tape removal test results for mice after administration of cmpd. 1 40 mg/kg 2 hours post reperfusion. At 4 weeks after stroke, 93.3% improvement compared to vehicle treatment (p<0.01).

### DETAILED DESCRIPTION

The lipoxygenases form a large family of enzymes capable of oxidizing arachidonic acid (AA) and related polyunsaturated fatty acids. In humans, in addition to 12/15-LOX, other members include 5-LOX, platelet-type-12-LOX (“P-12-LOX”), 12 (R)-LOX, epidermal LOX-3, and 15-LOX-2. The nomenclature of the lipoxygenase enzymes is based in part upon the carbon atom in AA that is oxidized with the aid of the enzyme. Correspondingly, 12/15-LOX can oxidize both C12 and C15 carbons of AA, forming 12- or 15-hydroperoxyeicosatetraenoic acid (12- or 15-HPETE), respectively. Lipoxygenases, including 12/15-LOX and its metabolites, are implicated in numerous disease states.

The present disclosure provides compounds containing a 2-(2,3,5-trisubstituted phenyl) oxazole structural moiety. These compounds are useful in treating or preventing pathological conditions associated with 12/15-LOX. Certain embodiments of the compounds and the 12/15-LOX-associated conditions are described in this disclosure. Pharmaceutical compositions, dosage forms, and combination treatments are also described.

### Therapeutic Compounds

In some embodiments, the present disclosure provides a compound of Formula (I):

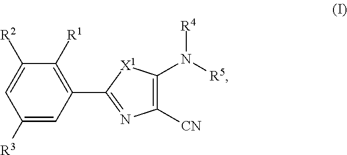

or a pharmaceutically acceptable salt thereof, wherein:
X¹ is selected from O and S;
R¹, R², and R³ are each independently selected from halo, CN, C₁₋₃ alkyl, C₁₋₃ haloalkyl, C₁₋₃ alkoxy, and C₁₋₃ haloalkoxy;
R⁴ is selected from H, C₁₋₃ alkyl, and HO—C₁₋₃ alkylene;
R⁵ is selected from C₁₋₆ alkyl, C₂₋₆ alkenyl, C₂₋₆ alkynyl, C(O)OR^{a1}, C(O)N(R^{a1})₂, P(═O)(OR^{a1})₂, and C(O)R^{b1}; wherein said C₁₋₆ alkyl, C₂₋₆ alkenyl, and C₂₋₆ alkynyl are each optionally substituted with a substituent selected from OR^{a1} and OP(═O)(OR^{a1})₂;
each R^{a1} is independently selected from H, C₁₋₆ alkyl, C₆₋₁₀ aryl, C₁₋₆ alkyl-C₆₋₁₀ aryl, and C₁₋₆ alkyl-C₆₋₁₀ aryl-C₁₋₆ alkyl, wherein said C₁₋₆ alkyl, C₆₋₁₀ aryl, C₁₋₆ alkyl-C₆₋₁₀ aryl, and C₁₋₆ alkyl-C₆₋₁₀ aryl-C₁₋₆ alkyl are each optionally substituted with a substituent selected from amino, C₁₋₆ alkylamino, (C₁₋₆ haloalkyl)amino, di(C₁₋₆ alkyl)amino, (C₁₋₆ alkyl) (C₁₋₆ haloalkyl)amino, (C₆₋₁₀ aryl)amino, (C₆₋₁₀ aryl) (C₁₋₆ alkyl)amino, (5-6-membered heteroaryl)amino, (5-6-membered heteroaryl) (C₁₋₆ alkyl)amino, C₆₋₁₀ aryl, 4-6 membered heterocycloalkyl, 5-6-membered heteroaryl, and OR^{a2}, wherein said C₆₋₁₀ aryl, 4-6 membered heterocycloalkyl, and 5-6-membered heteroaryl are each optionally substituted with 1, 2, or 3 substituents independently selected from amino, C₁₋₆ alkylamino, di(C₁₋₆ alkyl)amino, carboxy, and halo;
each R^{a2} is independently selected from H, C₁₋₃ alkyl, C₁₋₃ haloalkyl, C₁₋₃ alkoxy-C₁₋₃ alkyl, 4-7 membered heterocycloalkyl-C₁₋₃ alkyl, 5-6-membered heteroaryloxy-C₁₋₃ alkyl, C₆₋₁₀ aryl, and 5-6-membered heteroaryl, wherein said C₆₋₁₀ aryl and 5-6-membered heteroaryl are each optionally substituted with 1, 2, or 3 substituents independently selected from halo, C₁₋₃ alkoxy, C₁₋₃ haloalkoxy, C₁₋₃ alkyl, and C₁₋₃ haloalkyl; and
R^{b1} is C₁₋₆ alkyl, optionally substituted with a substituent selected from amino, C₁₋₆ alkylamino, di(C₁₋₆ alkyl)amino, and 4-7 membered heterocycloalkyl ring comprising at least one N atom.

In some embodiments, X¹ is O.

In some embodiments, X¹ is S.

In some embodiments, the compound has formula:

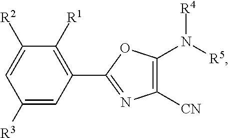

or a pharmaceutically acceptable salt thereof.

In some embodiments, the compound has formula:

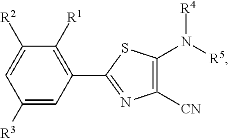

or a pharmaceutically acceptable salt thereof.

In some embodiments, R¹ is halo. In some embodiments, R¹ is CN. In some embodiments, R¹ is C₁₋₃ alkyl. In some embodiments, R¹ is C₁₋₃ haloalkyl.

In some embodiments, R² is halo. In some embodiments, R² is CN. In some embodiments, R² is C₁₋₃ alkyl. In some embodiments, R² is C₁₋₃ haloalkyl.

In some embodiments, R³ is halo. In some embodiments, R³ is CN. In some embodiments, R³ is C₁₋₃ alkyl. In some embodiments, R³ is C₁₋₃ haloalkyl.

In some embodiments, R¹, R², and R³ are each halo.

In some embodiments:
R² and R³ are each halo; and
R¹ is selected from C₁₋₃ alkyl, C₁₋₃ alkoxy, and C₁₋₃ haloalkoxy.

In some embodiments:
R¹ and R³ are each halo; and
R² is selected from C₁₋₃ alkyl, C₁₋₃ alkoxy, and C₁₋₃ haloalkyl.

In some embodiments:
R¹ and R² are each halo; and
R³ is selected from C₁₋₃ alkyl, C₁₋₃ haloalkyl, and CN.

In some embodiments, the compound has formula:

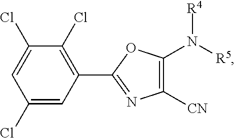

or a pharmaceutically acceptable salt thereof.

In some embodiments, R⁵ is C₁₋₃ alkyl.

In some embodiments, R⁵ is C₂₋₆ alkenyl, optionally substituted with OR^{a1}.

In some embodiments, R⁵ is C₂₋₆ alkynyl, optionally substituted with OR^{a1}.

In some embodiments, R⁵ is C₁₋₆ alkyl substituted with OR^{a1}.

In some embodiments, R⁵ is C₁₋₆ alkyl substituted with OP(═O)(OR^{a1})₂.

In some embodiments, R^{a1} is H.

In some embodiments, R^{a1} is C₁₋₆ alkyl, optionally substituted with C₆₋₁₀ aryl or OR^{a2}.

In some embodiments, R⁴ is H.

In some embodiments, R⁵ is C(O)OR^{a1}

In some embodiments, R^{a1} is C₁₋₆ alkyl, optionally substituted with a substituent selected from amino, C₁₋₆ alkylamino, and di(C₁₋₆ alkyl)amino.

In some embodiments, R⁵ is C(O)R^{b1}.

In some embodiments:
R⁴ is selected from H, C₁₋₃ alkyl, and HO—C₁₋₃ alkylene; and
R⁵ is selected from C₁₋₆ alkyl, C₂₋₆ alkenyl, and C₂₋₆ alkynyl, each of which is optionally substituted with a substituent selected from OR^{a1} and OP (═O) (OR^{a1})₂; and
each R^{a1} is independently selected from H and C₁₋₆ alkyl, wherein said C₁₋₆ alkyl is optionally substituted with a substituent selected from C₆₋₁₀ aryl and OR^{a2}.

In some embodiments:
R⁴ is selected from C(O)OR^{a1} and C(O)R^{b1};
R⁵ is C₁₋₃ alkyl; and
R^{a1} is C₁₋₆ alkyl, optionally substituted with a substituent selected from amino, C₁₋₆ alkylamino, and di(C₁₋₆ alkyl)amino.

In some embodiments, the compound of Formula (I) is selected from any one of the following compounds:

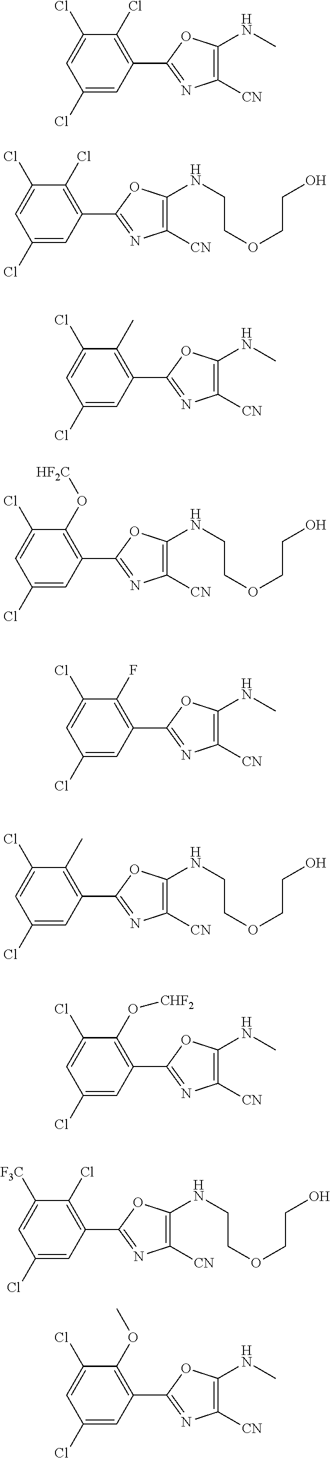

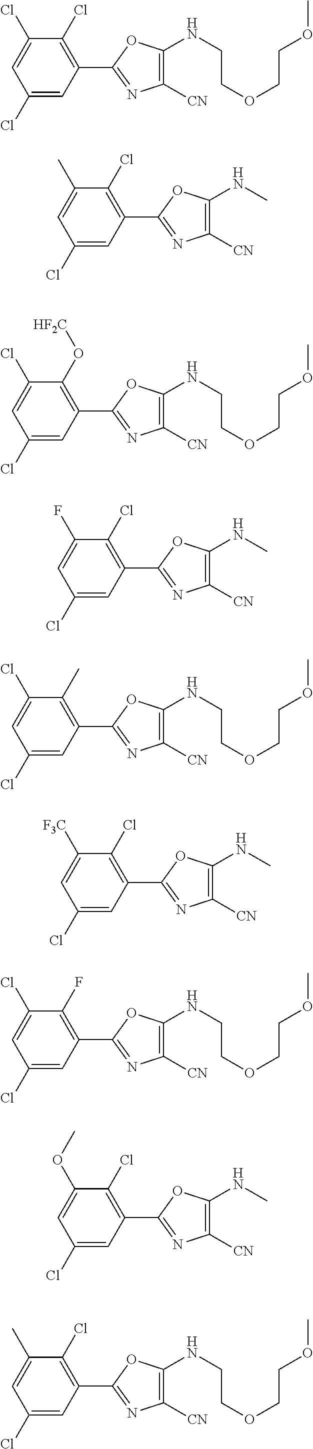

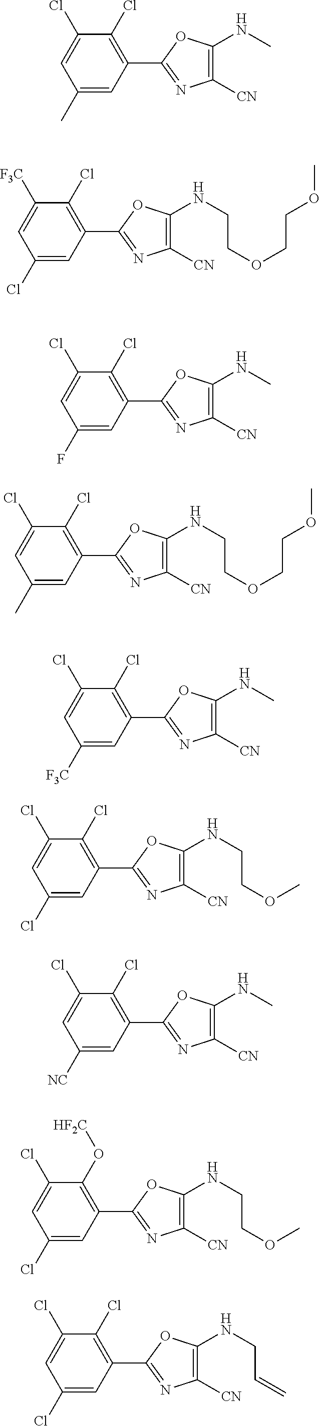

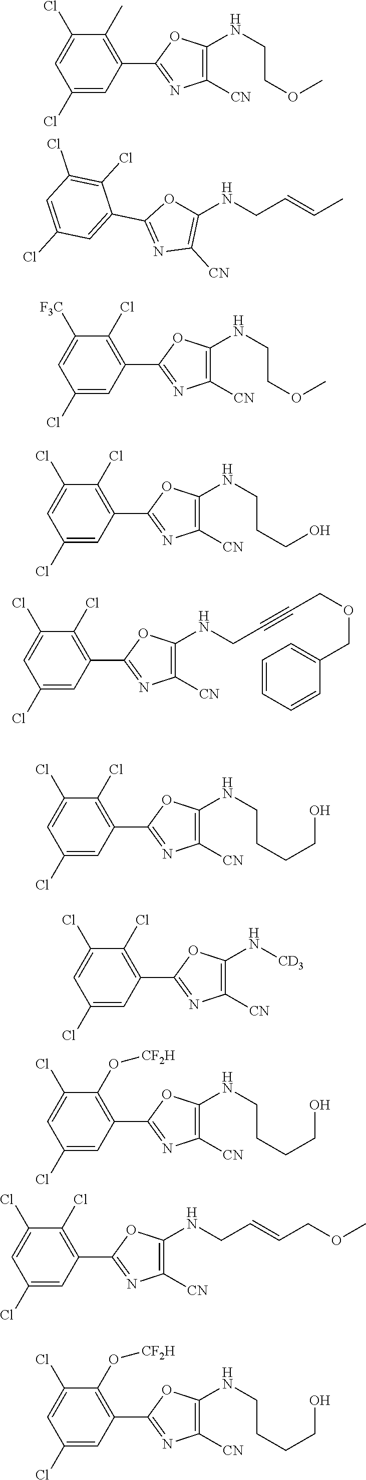

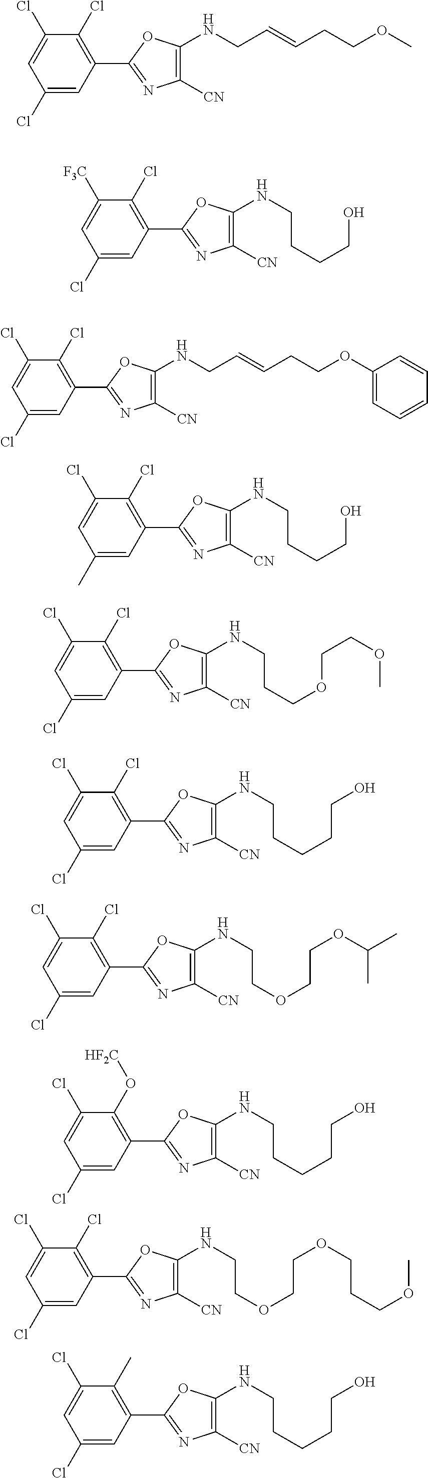

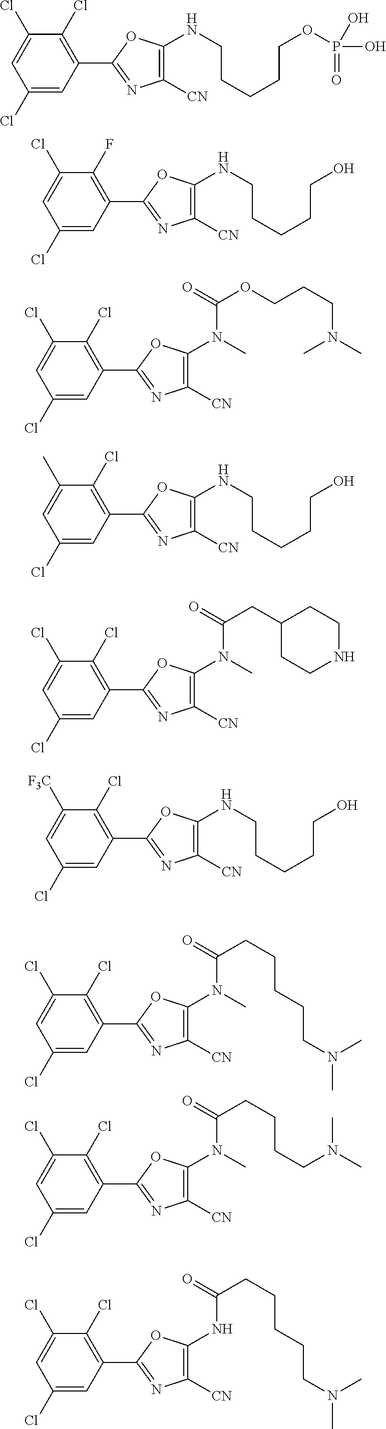

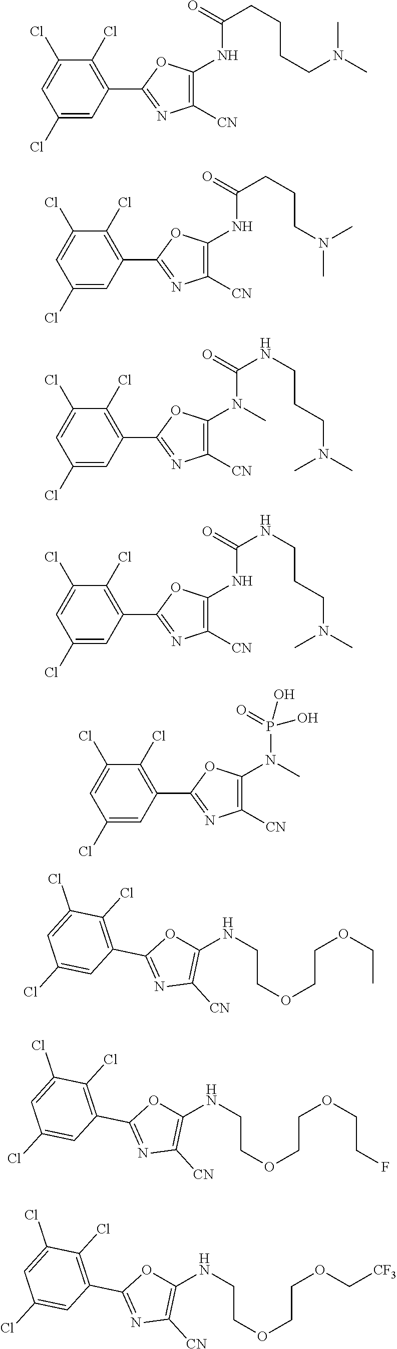

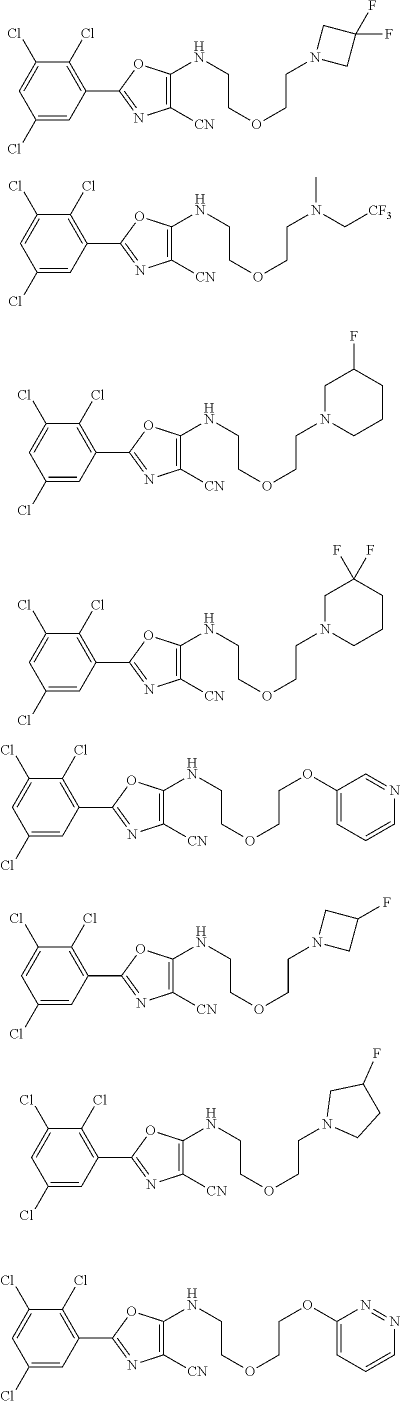

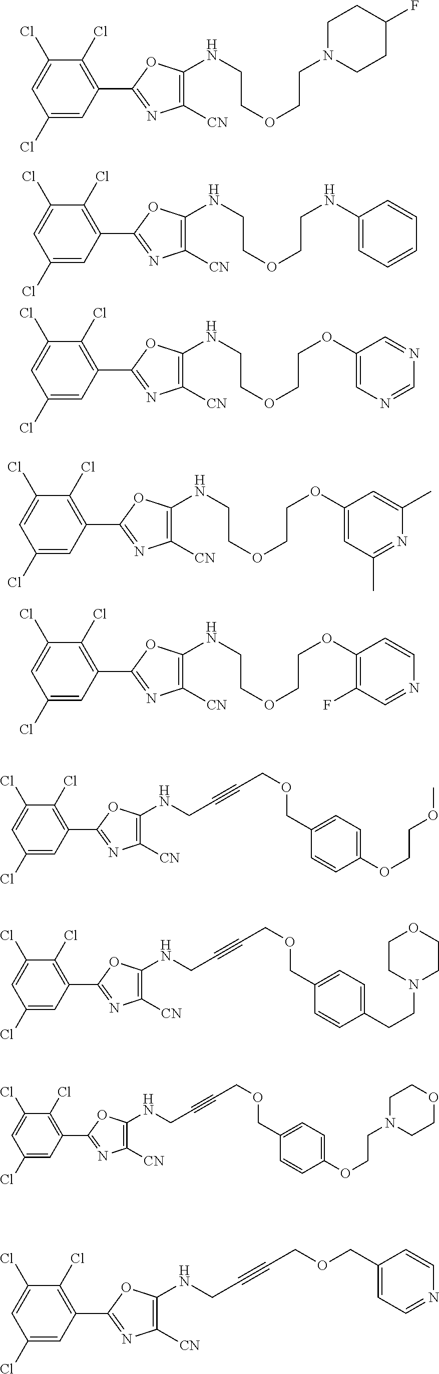

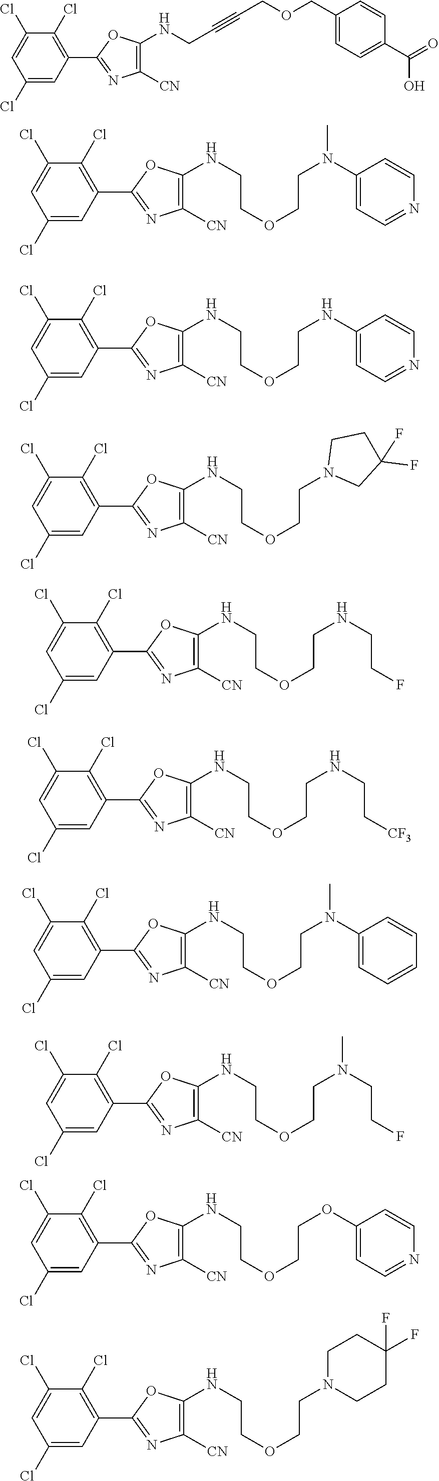

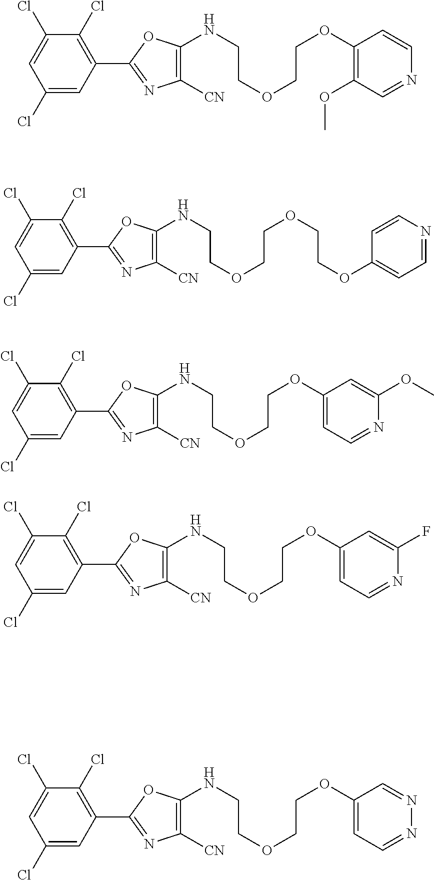

or a pharmaceutically acceptable salt thereof.

In some embodiments, the compound of Formula (I) is selected from any one of the following compounds:

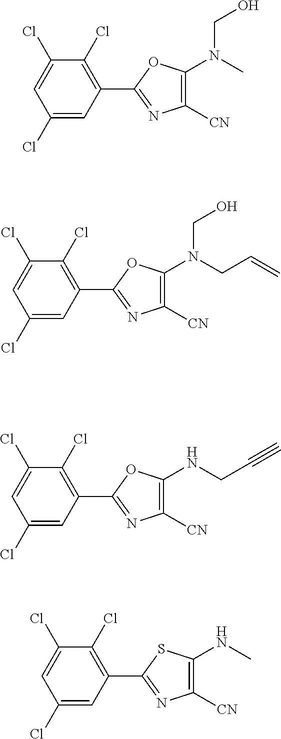 [SMILES: CN(CO)c1oc(-c2cc(Cl)cc(Cl)c2Cl)nc1C#N ; C=CCN(CO)c1oc(-c2cc(Cl)cc(Cl)c2Cl)nc1C#N ; C#CCNc1oc(-c2cc(Cl)cc(Cl)c2Cl)nc1C#N ; CNc1sc(-c2cc(Cl)cc(Cl)c2Cl)nc1C#N]

or a pharmaceutically acceptable salt thereof.

### Pharmaceutically Acceptable Salts

In some embodiments, a salt of a compound of Formula (I) is formed between an acid and a basic group of the compound, such as an amino functional group, or a base and an acidic group of the compound, such as a carboxyl functional group. According to another embodiment, the compound is a pharmaceutically acceptable acid addition salt.

In some embodiments, acids commonly employed to form pharmaceutically acceptable salts of the compounds of Formula (I) include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, ß-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid.

In some embodiments, bases commonly employed to form pharmaceutically acceptable salts of the compounds of Formula (I) include hydroxides of alkali metals, including sodium, potassium, and lithium; hydroxides of alkaline earth metals such as calcium and magnesium; hydroxides of other metals, such as aluminum and zinc; ammonia, organic amines such as unsubstituted or hydroxyl-substituted mono-, di-, or tri-alkylamines, dicyclohexylamine; tributyl amine; pyridine; N-methyl, N-ethylamine; diethylamine; triethylamine; mono-, bis-, or tris-(2-OH—(C1-C6)-alkylamine), such as N,N-dimethyl-N-(2-hydroxyethyl)amine or tri-(2-hydroxyethyl)amine; N-methyl-D-glucamine; morpholine; thiomorpholine; piperidine; pyrrolidine; and amino acids such as arginine, lysine, and the like.

In some embodiments, the compounds of Formula (I), or pharmaceutically acceptable salts thereof, are substantially isolated.

### Methods of Use

The compounds of the present disclosure advantageously and potently inhibit 12/15-LOX. As such, in some embodiments, the present disclosure provides a method of inhibiting 12/15-lipoxygenase (12/15-LOX) in a cell, the method comprising contacting the cell of a subject with an effective amount of a compound of the present disclosure, or a pharmaceutically acceptable salt thereof. In some embodiments, the cell is contacted in vitro, in vivo, or ex vivo. In some embodiments, the present disclosure provides a method of inhibiting 12/15-lipoxygenase (12/15-LOX) in a cell of a subject, the method comprising administering to the subject a therapeutically effective amount of a compound of the present disclosure, or a pharmaceutically acceptable salt thereof.

In some embodiments, the present disclosure provides a method of treating or preventing a disease or disorder in which 12/15-lipoxygenase (LOX) is implicated in the pathology, the method comprising administering to a subject (e.g., in need thereof) a therapeutically effective amount of any one of the compounds of the present disclosure, or a pharmaceutically acceptable salt thereof. Examples of such diseases or disorders include conditions that is worsened by the activity of 12/15-LOX and also conditions the progression of which is linked to the activity of 12/15-LOX. Without being bound by any theory, it is believed that such conditions can be therapeutically treated in a subject by inhibiting 12/15-LOX by the compounds of the present disclosure. The subject is said to be “in need thereof” (i.e., in need of treatment) when the need for treating or preventing the aforementioned disease or disorder in the subject is based on diagnosing the subject with the disease or disorder by a physician. Diagnostic methods for diagnosing the subject would be immediately apparent to a skilled physician. Such methods include, for example, visual observations, studying medical history, and physical exams, as well as various imaging techniques and laboratory tests. For example, a stroke, a brain injury, or an ischemic event can be diagnosed with the aid of neuroimaging (e.g., magnetic resonance imaging, computerized tomography, diffuse optical imaging, event-related optical signal, magnetoencephalography, positron emission tomography, or single-photon emission computed tomography).

Numerous scientific publications provide credible evidence that 12/15-LOX is implicated in pathology of a disease or condition selected from: diabetic retinopathy, non-alcoholic fatty liver disease (NAFLD), non-alcoholic steatohepatitis (NASH), stroke, diabetes, obesity, asthma, glomerulonephritis, osteoporosis, periventricular leukomalacia, cardiac arrest with resuscitation, atherosclerosis, neurodegenerative or neuroinflammatory disorders (e.g., Parkinson's disease, Alzheimer's disease, or dementia), cancer, brain injury, a disease involving hypoxia or anoxia, myocardial infarction, cardiovascular disease, heart failure (e.g., chronic or congestive heart failure), ischemia (e.g., cerebral ischemia, retinal ischemia, myocardial ischemia, or post-surgical cognitive dysfunction), inflammatory disease (e.g., arterial inflammation, inflammatory bowel disease, Crohn's disease, renal disease, asthma, allergic rhinitis, gout, cardiopulmonary inflammation, rheumatoid arthritis, osteoarthritis, muscle fatigue, acne, dermatitis, or psoriasis), chronic bronchitis, mucus hypersecretion, chronic obstructive pulmonary disease (COPD), pulmonary fibrosis (including fibrosis caused by chemotherapy), idiopathic pulmonary fibrosis, cystic fibrosis, adult respiratory distress syndrome, CNS disorders, psychiatric disorders (e.g., anxiety or depression), peripheral neuropathy (e.g., spinal cord injury, head injury, or surgical trauma), allograft tissue or organ transplant rejection, autoimmune disorder (e.g., eczema), and disorders involving bone loss or bone formation.

In some embodiments, the present disclosure provides a method of treating a stroke, the method comprising administering to a subject (e.g., in need thereof) a therapeutically effective amount of a compound of the present disclosure, or a pharmaceutically acceptable salt thereof. Strokes are sudden neurological disorders that occur when blood flow to the brain is disturbed. There are two kinds of strokes. One is called acute ischemic stroke (AIS), which is due to blood flow blockage. AIS is an episode of neurological dysfunction caused by focal brain, spinal cord, or retina ischemia with evidence of acute infarction. There are at least four different causes of blood flow interruption: (1) a blood clot in a blood vessel; (2) a blood clot in the dural venous sinuses, which drain blood from the brain; (3) an embolus clogging a blood vessel; or (4) a sudden drop in blood pressure. Stroke symptoms can and frequently do persist beyond 24 hours if the patient survives the initial damage. The other kind of stroke is called hemorrhagic stroke, which is caused by a weakened blood vessel that ruptures and bleeds into the surrounding brain tissue. A transient ischemic attack (TIA) is a condition mimicking AIS, in which a temporary interruption in blood flow to part of the brain results in impaired brain functions, but does not necessarily result in brain tissue damage. Without being bound by any theory, it is believed that oxidative stress, which including producing AA metabolites by LOX enzyme, contributes to the pathology of ischemic diseases such as stroke. Numerous scientific articles attest to increased activity of 12/15-LOX in neurons and brain vasculature during and after stroke. Without being bound by any theory, it is believed that LOX inhibitor compounds reduce infarct size, leakage or the blood brain barrier and edema formation, and hemorrhagic transformation following infusion of tPA. In some embodiments, the present disclosure provides a method of ameliorating symptoms of stroke (e.g., ischemic stroke). Suitable examples of such symptoms include sudden numbness, tingling, weakness, or loss of movement in the face, arm, or leg, especially on only one side of the body; sudden vision changes; sudden trouble speaking; sudden confusion or trouble understanding simple statements; sudden problems with walking or balance, and a sudden, severe headache that is different from past headaches. Other examples may include reducing infarct size in a permanent focal ischemia.

Further, with regard to stroke, the inhibitors of the present disclosure can be used as (i) a stand-alone treatment, either in an ambulance or after arrival at the hospital; (ii) in conjunction with tPA, the only drug currently approved by the FDA for acute stroke treatment; (iii) in conjunction with endovascular treatment with a stent retriever, to provide neuroprotection and protect the vasculature. The compounds also can be used to treat hemorrhagic forms of stroke, e.g., subarachnoid hemorrhage (SAH). The compounds could be administered orally or by parenteral delivery, for example through intravenous injection/infusion. In a similar matter, the compounds can be administered to the subject to treat other ischemic and/or lipoxygenase-related diseases, such as diabetes, diabetic retinopathy, liver diseases, and cancer.

In some embodiments, the 12/15-LOX inhibitors disclosed herein can be used to treat a stroke with hemorrhagic transformation. Hemorrhagic transformation refers to hemorrhages that develop inside areas of ischemia. For example, a subject who receives an oral anticoagulant and experiences a stroke, can be treated with the 12/15-LOX inhibitors disclosed herein. In some embodiments, the 12/15-LOX inhibitors disclosed herein can be administered as adjuvant to tPA. For the treatment of strokes, the 12/15-LOX inhibitors provided in this disclosure can be administered during the early phase of stroke or days (e.g., 1, 2, 3, 4, 5, 6, 7, 8, 9, 10, or more) after a stroke to aid in stroke recovery.

In some embodiments, the present disclosure provides a method of treating periventricular leukomalacia (PVL), the method comprising administering to a subject in need thereof a therapeutically acceptable amount of a compound of the present disclosure, or a pharmaceutically acceptable salt thereof. Scientific evidence clearly shows that PVL patients also feature increased 12/15-LOX. Periventricular leukomalacia is the most frequent cause of cerebral palsy in premature infants. This early neonatal disorder is due to the formation of single or multiple lesions of the ring of periventricular white matter, occurring during prenatal or neonatal life. Periventricular leukomalacia is responsible for the majority of motor sequelae of prematurity.

In some embodiments, the present disclosure provides a method of treating cancer, the method comprising administering to a subject (e.g., in need thereof) a therapeutically effective amount of a compound of the present disclosure, or a pharmaceutically acceptable salt thereof. Suitable examples of cancers include: prostate cancer, gastric cancer, breast cancer, pancreatic cancer, colorectal cancer, esophageal cancer, and airway carcinoma.

In some embodiments, the present disclosure provides a compound of the present disclosure, or a pharmaceutically acceptable salt thereof, for use in treating or preventing any of the diseases, disorders, or conditions disclosed herein.

In some embodiments, the present disclosure provides a pharmaceutical composition comprising a compound of the present disclosure, or a pharmaceutically acceptable salt thereof, and a pharmaceutically acceptable carrier, for use in treating or preventing any of the diseases, disorders, or conditions disclosed herein.

In some embodiments, the present disclosure provides use of a compound of the present disclosure, or a pharmaceutically acceptable salt thereof, or a pharmaceutical composition comprising same, in the manufacture of a medicament for the treatment of any one of the diseases, disorders, or conditions described herein.

### Pharmaceutical Compositions

The present application also provides pharmaceutical compositions comprising an effective amount of any of the compounds of the present disclosure, or a pharmaceutically acceptable salt thereof; and a pharmaceutically acceptable carrier. The pharmaceutical composition may also comprise any one of the additional therapeutic agents described herein. In certain embodiments, the application also provides pharmaceutical compositions and dosage forms comprising any one the additional therapeutic agents described herein. The carrier(s) are “acceptable” in the sense of being compatible with the other ingredients of the formulation and, in the case of a pharmaceutically acceptable carrier, not deleterious to the recipient thereof in an amount used in the medicament.

Pharmaceutically acceptable carriers, adjuvants and vehicles that may be used in the pharmaceutical compositions of the present application include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

The compositions or dosage forms may contain any one of the compounds and therapeutic agents described herein in the range of 0.005% to 100% with the balance made up from the suitable pharmaceutically acceptable excipients. The contemplated compositions may contain 0.001%-100% of any one of the compounds and therapeutic agents provided herein, in one embodiment 0.1-95%, in another embodiment 75-85%, in a further embodiment 20-80%, wherein the balance may be made up of any pharmaceutically acceptable excipient described herein, or any combination of these excipients.

### Routes of Administration and Dosage Forms

The pharmaceutical compositions of the present application include those suitable for any acceptable route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intranasal, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal.

Compositions and formulations described herein may conveniently be presented in a unit dosage form, e.g., tablets, sustained release capsules, and in liposomes, and may be prepared by any methods well known in the art of pharmacy. See, for example, Remington: The Science and Practice of Pharmacy, Lippincott Williams & Wilkins, Baltimore, MD (20th ed. 2000). Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product.

In some embodiments, any one of the compounds and therapeutic agents disclosed herein are administered orally. Compositions of the present application suitable for oral administration may be presented as discrete units such as capsules, sachets, granules or tablets each containing a predetermined amount (e.g., effective amount) of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption. In the case of tablets for oral use, carriers that are commonly used include lactose, sucrose, glucose, mannitol, and silicic acid and starches. Other acceptable excipients may include: a) fillers or extenders such as starches, lactose, sucrose, glucose, mannitol, and silicic acid, b) binders such as, for example, carboxymethylcellulose, alginates, gelatin, polyvinylpyrrolidone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. For oral administration in a capsule form, useful diluents include lactose and dried corn starch. When aqueous suspensions are administered orally, the active ingredient is combined with emulsifying and suspending agents. If desired, certain sweetening and/or flavoring and/or coloring agents may be added. Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions or infusion solutions which may contain antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, saline (e.g., 0.9% saline solution) or 5% dextrose solution, immediately prior to use. Extemporaneous injection solutions and suspensions may be prepared from sterile powders, granules and tablets. The injection solutions may be in the form, for example, of a sterile injectable aqueous or oleaginous suspension. This suspension may be formulated according to techniques known in the art using suitable dispersing or wetting agents and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical compositions of the present application may be administered in the form of suppositories for rectal administration. These compositions can be prepared by mixing a compound of the present application with a suitable non-irritating excipient which is solid at room temperature but liquid at the rectal temperature and therefore will melt in the rectum to release the active components. Such materials include, but are not limited to, cocoa butter, beeswax, and polyethylene glycols.

The pharmaceutical compositions of the present application may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. See, for example, U.S. Pat. No. 6,803,031. Additional formulations and methods for intranasal administration are found in Ilium, L., J Pharm Pharmacol, 56:3-17, 2004 and Ilium, L., Eur J Pharm Sci 11:1-18, 2000.

The topical compositions of the present disclosure can be prepared and used in the form of an aerosol spray, cream, emulsion, solid, liquid, dispersion, foam, oil, gel, hydrogel, lotion, mousse, ointment, powder, patch, pomade, solution, pump spray, stick, towelette, soap, or other forms commonly employed in the art of topical administration and/or cosmetic and skin care formulation. The topical compositions can be in an emulsion form. Topical administration of the pharmaceutical compositions of the present application is especially useful when the desired treatment involves areas or organs readily accessible by topical application. In some embodiments, the topical composition comprises a combination of any one of the compounds and therapeutic agents disclosed herein, and one or more additional ingredients, carriers, excipients, or diluents including, but not limited to, absorbents, anti-irritants, anti-acne agents, preservatives, antioxidants, coloring agents/pigments, emollients (moisturizers), emulsifiers, film-forming/holding agents, fragrances, leave-on exfoliants, prescription drugs, preservatives, scrub agents, silicones, skin-identical/repairing agents, slip agents, sunscreen actives, surfactants/detergent cleansing agents, penetration enhancers, and thickeners.

The compounds and therapeutic agents of the present application may be incorporated into compositions for coating an implantable medical device, such as prostheses, artificial valves, vascular grafts, stents, or catheters. Suitable coatings and the general preparation of coated implantable devices are known in the art and are exemplified in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition. Coatings for invasive devices are to be included within the definition of pharmaceutically acceptable carrier, adjuvant or vehicle, as those terms are used herein.

According to another embodiment, the present application provides an implantable drug release device impregnated with or containing a compound or a therapeutic agent, or a composition comprising a compound of the present application or a therapeutic agent, such that said compound or therapeutic agent is released from said device and is therapeutically active.

### Dosages and Regimens

In the pharmaceutical compositions of the present application, any of the compounds of the present disclosure is present in an effective amount (e.g., a therapeutically effective amount). Effective doses may vary, depending on the diseases treated, the severity of the disease, the route of administration, the sex, age and general health condition of the subject, excipient usage, the possibility of co-usage with other therapeutic treatments such as use of other agents and the judgment of the treating physician.

In some embodiments, an effective amount of any of the compounds of the present disclosure can range, for example, from about 0.001 mg/kg to about 500 mg/kg (e.g., from about 0.001 mg/kg to about 200 mg/kg; from about 0.01 mg/kg to about 200 mg/kg; from about 0.01 mg/kg to about 150 mg/kg; from about 0.01 mg/kg to about 100 mg/kg; from about 0.01 mg/kg to about 50 mg/kg; from about 0.01 mg/kg to about 10 mg/kg; from about 0.01 mg/kg to about 5 mg/kg; from about 0.01 mg/kg to about 1 mg/kg; from about 0.01 mg/kg to about 0.5 mg/kg; from about 0.01 mg/kg to about 0.1 mg/kg; from about 0.1 mg/kg to about 200 mg/kg; from about 0.1 mg/kg to about 150 mg/kg; from about 0.1 mg/kg to about 100 mg/kg; from about 0.1 mg/kg to about 50 mg/kg; from about 0.1 mg/kg to about 10 mg/kg; from about 0.1 mg/kg to about 5 mg/kg; from about 0.1 mg/kg to about 2 mg/kg; from about 0.1 mg/kg to about 1 mg/kg; or from about 0.1 mg/kg to about 0.5 mg/kg). In some embodiments, an effective amount of a compound is about 0.1 mg/kg, about 0.5 mg/kg, about 1 mg/kg, about 2 mg/kg, or about 5 mg/kg.

The foregoing dosages can be administered on a daily basis (e.g., as a single dose or as two or more divided doses, e.g., once daily, twice daily, thrice daily) or non-daily basis (e.g., every other day, every two days, every three days, once weekly, twice weekly, once every two weeks, once a month).

### Kits

The present invention also includes pharmaceutical kits useful, for example, in the treatment of disorders, diseases and conditions referred to herein, which include one or more containers containing a pharmaceutical composition comprising a therapeutically effective amount of a compound of the present disclosure. Such kits can further include, if desired, one or more of various conventional pharmaceutical kit components, such as, for example, containers with one or more pharmaceutically acceptable carriers, additional containers, etc. Instructions, either as inserts or as labels, indicating quantities of the components to be administered, guidelines for administration, and/or guidelines for mixing the components, can also be included in the kit. The kit may optionally include an additional therapeutic agent as described herein.

### Combinations

The compounds of the present disclosure can be used on combination with at least one medication or therapy useful, e.g., in treating or alleviating symptoms of 12/15-LOX-associated disorder. Suitable examples of such medications include various medications useful in treating stroke. For example, an antithrombotic agent, or a pharmaceutically acceptable salt thereof. Antithrombotic agents are further divided into the following three subtypes: anticoagulants, antiplatelet drugs, and thrombolytic drugs. Suitable examples of anticoagulants include: coumarins, heparin, warfarin, acenocoumarol, phenprocoumon, atromentin, phenindione, fondaparinux, idraparinux, direct factor Xa inhibitors, direct thrombin inhibitors, antithrombin protein therapeutics, batroxobin, and hementin. Suitable examples of antiplatelet drugs include: irreversible cyclooxygenase inhibitors (e.g., aspirin or triflusal), adenosine diphosphate receptor inhibitors (e.g., clopidogrel, prasugrel, ticagrelor, or ticlopidine), phosphodiesterase inhibitors (e.g., cilostazol), glycoprotein IIB/IIIA inhibitors (e.g., abciximab, eptifibatide, or tirofiban), adenosine reuptake inhibitors (e.g., dipyridamole), and thromboxane inhibitors (e.g., thromboxane synthase inhibitors or thromboxane receptor antagonists). Suitable examples of thrombolytic drugs include: tissue plasminogen activator t-PA (e.g., alteplase, reteplase, or tenecteplase), anistreplase, streptokinase, and urokinase. An ischemic stroke can also be treated by endovascular procedures, in which a catheter is sent to the blood flow blockage site to remove the blood clot. t-PA can be optionally administered during the endovascular procedures. The compound of the present disclosure may be administered to the patient simultaneously with the additional therapeutic agent (in the same pharmaceutical composition or dosage form or in different compositions or dosage forms) or consecutively (the additional therapeutic agent may be administered in a separate pharmaceutical composition or dosage form before or after administration of the compound of the present disclosure).

### Definitions

As used herein, the term “about” means “approximately” (e.g., plus or minus approximately 10% of the indicated value).

At various places in the present specification, substituents of compounds of the invention are disclosed in groups or in ranges. It is specifically intended that the invention include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl. In some embodiments, any alkyl group within any compound of this disclosure may contain at least one deuterium (“D”) atom.

At various places in the present specification various aryl, heteroaryl, cycloalkyl, and heterocycloalkyl rings are described. Unless otherwise specified, these rings can be attached to the rest of the molecule at any ring member as permitted by valency. For example, the term “a pyridine ring” or “pyridinyl” may refer to a pyridin-2-yl, pyridin-3-yl, or pyridin-4-yl ring.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

As used herein, the phrase “optionally substituted” means unsubstituted or substituted. The substituents are independently selected, and substitution may be at any chemically accessible position. As used herein, the term “substituted” means that a hydrogen atom is removed and replaced by a substituent. A single divalent substituent, e.g., oxo, can replace two hydrogen atoms. It is to be understood that substitution at a given atom is limited by valency.

Throughout the definitions, the term “Cₙ₋ₘ” indicates a range which includes the endpoints, wherein n and m are integers and indicate the number of carbons. Examples include C₁₋₄, C₁₋₆, and the like.

As used herein, the term “Cₙ₋ₘ alkyl”, employed alone or in combination with other terms, refers to a saturated hydrocarbon group that may be straight-chain or branched, having n to m carbons. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms. In some embodiments, any alkyl group within any compound of this disclosure may contain at least one deuterium (“D”) atom.

As used herein, the term “Cₙ₋ₘ haloalkyl”, employed alone or in combination with other terms, refers to an alkyl group having from one halogen atom to 2s+1 halogen atoms which may be the same or different, where “s” is the number of carbon atoms in the alkyl group, wherein the alkyl group has n to m carbon atoms. In some embodiments, the haloalkyl group is fluorinated only. In some embodiments, the alkyl group has 1 to 6, 1 to 4, or 1 to 3 carbon atoms.

As used herein, “Cₙ₋ₘ alkenyl” refers to an alkyl group having one or more double carbon-carbon bonds and having n to m carbons. Example alkenyl groups include, but are not limited to, ethenyl, n-propenyl, isopropenyl, n-butenyl, sec-butenyl, and the like. In some embodiments, the alkenyl moiety contains 2 to 6, 2 to 4, or 2 to 3 carbon atoms.

As used herein, “Cₙ₋ₘ alkynyl” refers to an alkyl group having one or more triple carbon-carbon bonds and having n to m carbons. Example alkynyl groups include, but are not limited to, ethynyl, propyn-1-yl, propyn-2-yl, and the like. In some embodiments, the alkynyl moiety contains 2 to 6, 2 to 4, or 2 to 3 carbon atoms.

As used herein, the term “Cₙ₋ₘ alkylene”, employed alone or in combination with other terms, refers to a divalent alkyl linking group having n to m carbons. Examples of alkylene groups include, but are not limited to, ethan-1,1-diyl, ethan-1,2-diyl, propan-1,1,-diyl, propan-1,3-diyl, propan-1,2-diyl, butan-1,4-diyl, butan-1,3-diyl, butan-1,2-diyl, 2-methyl-propan-1,3-diyl, and the like. In some embodiments, the alkylene moiety contains 2 to 6, 2 to 4, 2 to 3, 1 to 6, 1 to 4, or 1 to 2 carbon atoms.

As used herein, the term “Cₙ₋ₘ alkoxy”, employed alone or in combination with other terms, refers to a group of formula-O-alkyl, wherein the alkyl group has n to m carbons. Example alkoxy groups include, but are not limited to, methoxy, ethoxy, propoxy (e.g., n-propoxy and isopropoxy), butoxy (e.g., n-butoxy and tert-butoxy), and the like. In some embodiments, the alkyl group has 1 to 6, 1 to 4, or 1 to 3 carbon atoms.

As used herein, “Cₙ₋ₘ haloalkoxy” refers to a group of formula-O-haloalkyl having n to m carbon atoms. An example haloalkoxy group is OCF₃. In some embodiments, the haloalkoxy group is fluorinated only. In some embodiments, the alkyl group has 1 to 6, 1 to 4, or 1 to 3 carbon atoms.

As used herein, the term “amino” refers to a group of formula —NH₂.

As used herein, the term “Cₙ₋ₘ alkylamino” refers to a group of formula-NH (alkyl), wherein the alkyl group has n to m carbon atoms. In some embodiments, the alkyl group has 1 to 6, 1 to 4, or 1 to 3 carbon atoms. Examples of alkylamino groups include, but are not limited to, N-methylamino, N-ethylamino, N-propylamino (e.g., N-(n-propyl)amino and N-isopropylamino), N-butylamino (e.g., N-(n-butyl)amino and N-(tert-butyl)amino), and the like.

As used herein, the term “di(Cₙ₋ₘ-alkyl)amino” refers to a group of formula-N (alkyl)₂, wherein the two alkyl groups each has, independently, n to m carbon atoms. In some embodiments, each alkyl group independently has 1 to 6, 1 to 4, or 1 to 3 carbon atoms.

As used herein, “halo” refers to F, Cl, Br, or I. In some embodiments, a halo is F, Cl, or Br.

As used herein, “hydrogen,” or “H” refers to any stable isotope of the chemical element having only one proton in the nucleus. These isotopes include hydrogen-1 (one proton and zero neutrons in the nucleus), hydrogen-2 (also known as deuterium, or D, having one proton and one neutron in the nucleus), and hydrogen-3 (also known as tritium, or T, having one proton and two neutrons in the nucleus). Unless otherwise stated, when a position is designated specifically as “D” or “deuterium”, the position is understood to have deuterium at an abundance that is at least 3340 times greater than the natural abundance of deuterium, which is 0.015% (i.e., at least 50.1% incorporation of deuterium). In some embodiments, a compound of this disclosure has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

As used herein, “heterocycloalkyl” refers to non-aromatic monocyclic or polycyclic heterocycles having one or more ring-forming heteroatoms selected from O, N, or S. Included in heterocycloalkyl are monocyclic 4-, 5-, 6-, 7-, 8-, 9- or 10-membered heterocycloalkyl groups. Heterocycloalkyl groups can also include spirocycles. Example heterocycloalkyl groups include pyrrolidin-2-one, 1,3-isoxazolidin-2-one, pyranyl, tetrahydropuran, oxetanyl, azetidinyl, morpholino, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, benzazapene, and the like. Ring-forming carbon atoms and heteroatoms of a heterocycloalkyl group can be optionally substituted by 1 or 2 independently selected oxo or sulfido groups (e.g., C(O), S(O), C(S), or S(O)₂, etc.). The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. Also included in the definition of heterocycloalkyl are moieties that have one or more aromatic rings fused (i.e., having a bond in common with) to the cycloalkyl ring, for example, benzo or thienyl derivatives of piperidine, morpholine, azepine, etc. A heterocycloalkyl group containing a fused aromatic ring can be attached through any ring-forming atom including a ring-forming atom of the fused aromatic ring. In some embodiments, the heterocycloalkyl is a monocyclic 4-6 membered heterocycloalkyl having 1 or 2 heteroatoms independently selected from nitrogen, oxygen, or sulfur and having one or more oxidized ring members. In some embodiments, the heterocycloalkyl is a monocyclic or bicyclic 4-10 membered heterocycloalkyl having 1, 2, 3, or 4 heteroatoms independently selected from nitrogen, oxygen, or sulfur and having one or more oxidized ring members.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas a pyridin-3-yl ring is attached at the 3-position.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, N═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms. In some embodiments, the compound has the (R)-configuration. In some embodiments, the compound has the(S)-configuration.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “contacting” refers to the bringing together of indicated moieties in an in vitro system or an in vivo system. For example, “contacting” the 12/15-LOX with a compound of the invention includes the administration of a compound of the present invention to an individual or patient, such as a human, having 12/15-LOX, as well as, for example, introducing a compound of the invention into a sample containing a cellular or purified preparation containing the 12/16-LOX.

As used herein, the term “individual”, “patient”, or “subject” used interchangeably, refers to any animal, including mammals, preferably mice, rats, other rodents, rabbits, dogs, cats, swine, cattle, sheep, horses, or primates, and most preferably humans.

As used herein, the phrase “effective amount” or “therapeutically effective amount” refers to the amount of active compound or pharmaceutical agent that elicits the biological or medicinal response in a tissue, system, animal, individual or human that is being sought by a researcher, veterinarian, medical doctor or other clinician.

As used herein the term “treating” or “treatment” refers to 1) inhibiting the disease; for example, inhibiting a disease, condition or disorder in an individual who is experiencing or displaying the pathology or symptomatology of the disease, condition or disorder (i.e., arresting further development of the pathology and/or symptomatology), or 2) ameliorating the disease; for example, ameliorating a disease, condition or disorder in an individual who is experiencing or displaying the pathology or symptomatology of the disease, condition or disorder (i.e., reversing the pathology and/or symptomatology). As used herein, the term “preventing” or “prevention” of a disease, condition or disorder refers to decreasing the risk of occurrence of the disease, condition or disorder in a subject or group of subjects (e.g., a subject or group of subjects predisposed to or susceptible to the disease, condition or disorder). In some embodiments, preventing a disease, condition or disorder refers to decreasing the possibility of acquiring the disease, condition or disorder and/or its associated symptoms. In some embodiments, preventing a disease, condition or disorder refers to completely or almost completely stopping the disease, condition or disorder from occurring.

### EXAMPLES

### Example 1—Biological Activity of Exemplified Compounds as Inhibitors of 12/15-LOX

Assay: Compounds are dissolved in DMSO and diluted to 10 mM concentration in DMSO immediately before IC₅₀ analysis. To determine inhibition of the enzyme, 10 μL of DMSO was added to the first cuvette, and 10 μL of test compound stock solution was added to the second cuvette. Enzyme was then added to the cuvettes. The IC₅₀ experiment was performed for the following test compound concentrations: 20, 10, 3, 1, 0.3, 0.1, 0.03, and 0.01 μM. If the test compound was more potent than 0.3 μM, the concentrations used in the IC₅₀ determination were shifted to lower values such as: 3, 1, 0.3, 0.1, 0.03, 0.01, 0.003, and 0.001 μM. The % inhibition for each test compound concentration were calculated as follows:

$$\% Inhibition = 100 * ( 1 - \frac{Inhibitor Rate}{V e hicle Rate} )$$

| | | h12/15- | |
|---|---|---|---|
| | | LOX | |
| Cmpd. | | IC₅₀, | Max % |
| no. | Cmpd structure | μM | inhibition |
| 1 | 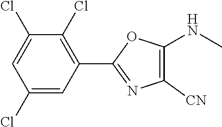 [SMILES: CNc1oc(-c2cc(Cl)cc(Cl)c2Cl)nc1C#N] | 0.12 | 94 |
| 2 | 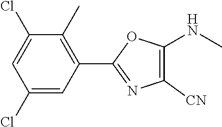 [SMILES: CNc1oc(-c2cc(Cl)cc(Cl)c2C)nc1C#N] | 0.063 | 85 |
| 3 | 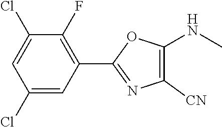 [SMILES: CNc1oc(-c2cc(Cl)cc(Cl)c2F)nc1C#N] | 0.33 | 88 |
| 4 | 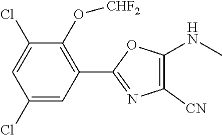 [SMILES: CNc1oc(-c2cc(Cl)cc(Cl)c2OC(F)F)nc1C#N] | 0.097 | 67 |
| 5 | 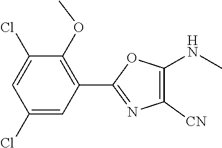 [SMILES: CNc1oc(-c2cc(Cl)cc(Cl)c2OC)nc1C#N] | 0.18 | 92 |
| 6 | 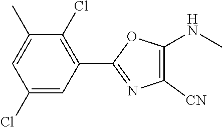 [SMILES: CNc1oc(-c2cc(Cl)cc(C)c2Cl)nc1C#N] | 0.18 | 99 |
| 7 | 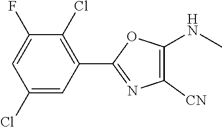 [SMILES: CNc1oc(-c2cc(Cl)cc(F)c2Cl)nc1C#N] | >20 | 37 |
| 8 | 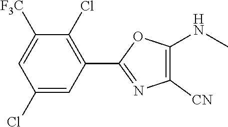 [SMILES: CNc1oc(-c2cc(Cl)cc(C(F)(F)F)c2Cl)nc1C#N] | 0.081 | 94 |
| 9 | 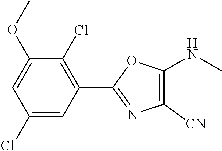 [SMILES: CNc1oc(-c2cc(Cl)cc(OC)c2Cl)nc1C#N] | 0.38 | 71 |
| 10 | 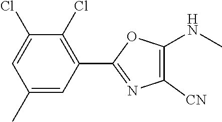 [SMILES: CNc1oc(-c2cc(C)cc(Cl)c2Cl)nc1C#N] | 0.38 | 80 |
| 11 | 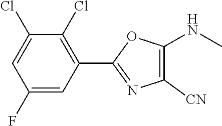 [SMILES: CNc1oc(-c2cc(F)cc(Cl)c2Cl)nc1C#N] | 0.87 | 93 |
| 12 | 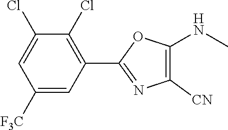 [SMILES: CNc1oc(-c2cc(C(F)(F)F)cc(Cl)c2Cl)nc1C#N] | >20 | 41 |
| 13 | 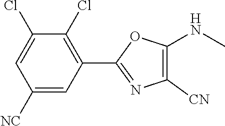 [SMILES: CNc1oc(-c2cc(C#N)cc(Cl)c2Cl)nc1C#N] | 1.1 | 100 |
| 14 | 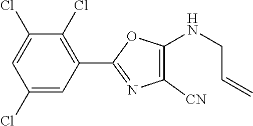 [SMILES: C=CCNc1oc(-c2cc(Cl)cc(Cl)c2Cl)nc1C#N] | 0.11 | 88 |
| 15 | 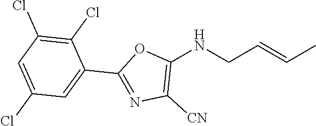 [SMILES: C/C=C/CNc1oc(-c2cc(Cl)cc(Cl)c2Cl)nc1C#N] | 0.17 | 92 |
| 16 | 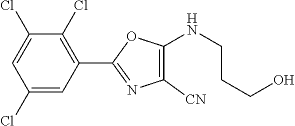 [SMILES: N#Cc1nc(-c2cc(Cl)cc(Cl)c2Cl)oc1NCCCO] | 4.3 | 100 |
| 17 | 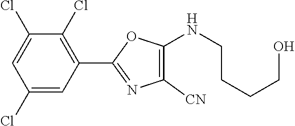 [SMILES: N#Cc1nc(-c2cc(Cl)cc(Cl)c2Cl)oc1NCCCCO] | 1.9 | 100 |
| 18 | 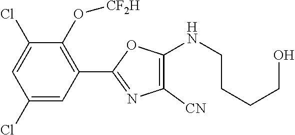 [SMILES: N#Cc1nc(-c2cc(Cl)cc(Cl)c2OC(F)F)oc1NCCCCO] | 2.0 | 90 |
| 19 | 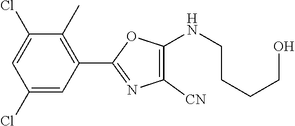 [SMILES: Cc1c(Cl)cc(Cl)cc1-c1nc(C#N)c(NCCCCO)o1] | 0.46 | 68 |
| 20 | 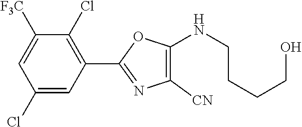 [SMILES: N#Cc1nc(-c2cc(Cl)cc(C(F)(F)F)c2Cl)oc1NCCCCO] | 1.5 | 97 |
| 21 | 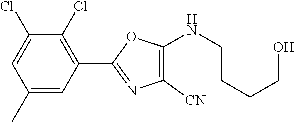 [SMILES: Cc1cc(Cl)c(Cl)c(-c2nc(C#N)c(NCCCCO)o2)c1] | 8.0 | 86 |
| 22 | 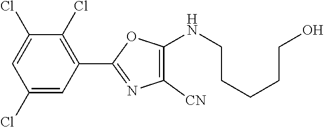 [SMILES: N#Cc1nc(-c2cc(Cl)cc(Cl)c2Cl)oc1NCCCCCO] | 0.51 | 82 |
| 23 | 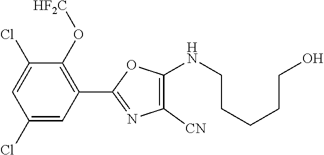 | 2.0 | 97 |
| 24 | 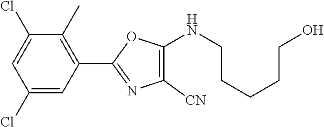 [SMILES: Cc1c(Cl)cc(Cl)cc1-c1nc(C#N)c(NCCCCCO)o1] | 0.32 | 80 |
| 25 | 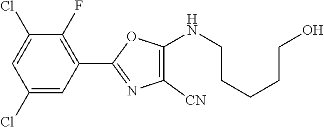 [SMILES: N#Cc1nc(-c2cc(Cl)cc(Cl)c2F)oc1NCCCCCO] | >20 | 38 |
| 26 | 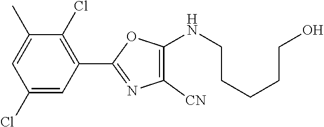 [SMILES: Cc1cc(Cl)cc(-c2nc(C#N)c(NCCCCCO)o2)c1Cl] | 0.80 | 100 |
| 27 | 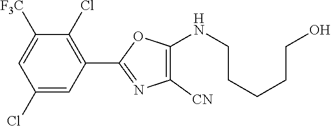 [SMILES: N#Cc1nc(-c2cc(Cl)cc(C(F)(F)F)c2Cl)oc1NCCCCCO] | 0.55 | 70 |
| 28 | 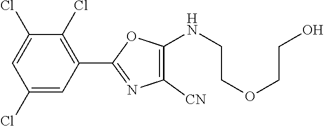 [SMILES: N#Cc1nc(-c2cc(Cl)cc(Cl)c2Cl)oc1NCCOCCO] | 1.5 | 87 |
| 29 | 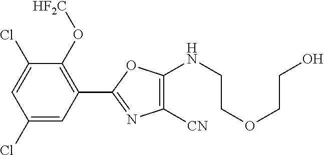 | 3.0 | 100 |
| 30 | 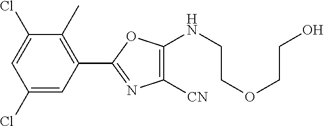 [SMILES: Cc1c(Cl)cc(Cl)cc1-c1nc(C#N)c(NCCOCCO)o1] | 0.67 | 100 |
| 31 | 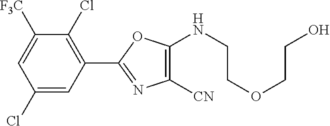 [SMILES: N#Cc1nc(-c2cc(Cl)cc(C(F)(F)F)c2Cl)oc1NCCOCCO] | 2.4 | 99 |
| 32 | 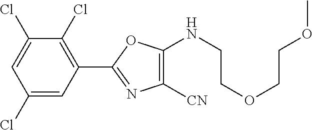 [SMILES: COCCOCCNc1oc(-c2cc(Cl)cc(Cl)c2Cl)nc1C#N] | 0.61 | 96 |
| 33 | 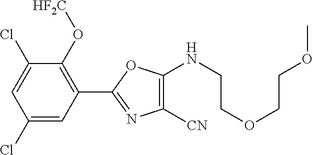 | 0.65 | 100 |
| 34 | 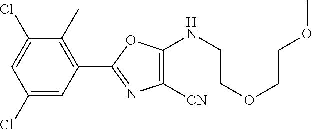 [SMILES: COCCOCCNc1oc(-c2cc(Cl)cc(Cl)c2C)nc1C#N] | 0.18 | 100 |
| 35 | 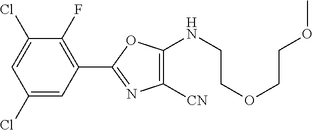 [SMILES: COCCOCCNc1oc(-c2cc(Cl)cc(Cl)c2F)nc1C#N] | 3.0 | 100 |
| 36 | 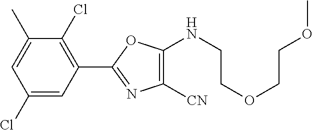 [SMILES: COCCOCCNc1oc(-c2cc(Cl)cc(C)c2Cl)nc1C#N] | 1.1 | 100 |
| 37 | 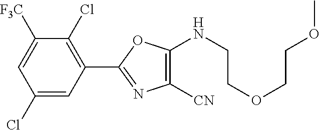 [SMILES: COCCOCCNc1oc(-c2cc(Cl)cc(C(F)(F)F)c2Cl)nc1C#N] | 1.8 | 96 |
| 38 | 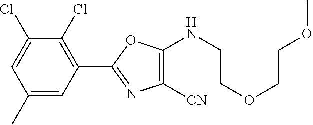 [SMILES: COCCOCCNc1oc(-c2cc(C)cc(Cl)c2Cl)nc1C#N] | 6.6 | 100 |
| 39 | 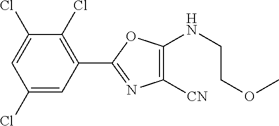 [SMILES: COCCNc1oc(-c2cc(Cl)cc(Cl)c2Cl)nc1C#N] | 0.37 | 100 |
| 40 | 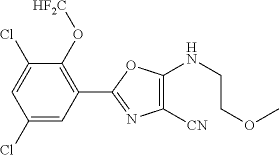 | 0.29 | 94 |
| 41 | 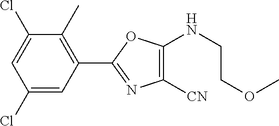 [SMILES: COCCNc1oc(-c2cc(Cl)cc(Cl)c2C)nc1C#N] | 0.17 | 89 |
| 42 | 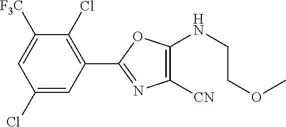 [SMILES: COCCNc1oc(-c2cc(Cl)cc(C(F)(F)F)c2Cl)nc1C#N] | 0.35 | 75 |
| 43 | 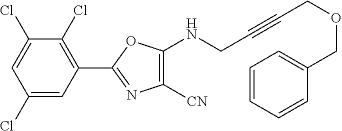 [SMILES: N#Cc1nc(-c2cc(Cl)cc(Cl)c2Cl)oc1NCC#CCOCc1ccccc1] | 0.23 | 93 |
| 44 | 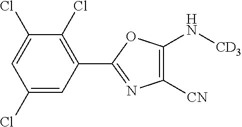 [SMILES: [2H]C([2H])([2H])Nc1oc(-c2cc(Cl)cc(Cl)c2Cl)nc1C#N] | 0.09 | 91 |
| 45 | 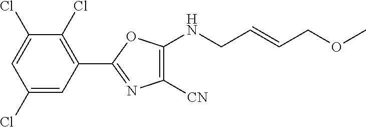 [SMILES: COC/C=C/CNc1oc(-c2cc(Cl)cc(Cl)c2Cl)nc1C#N] | 0.19 | 98 |
| 46 | 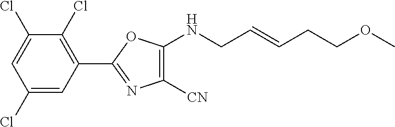 [SMILES: COCC/C=C/CNc1oc(-c2cc(Cl)cc(Cl)c2Cl)nc1C#N] | 0.21 | 95 |
| 47 | 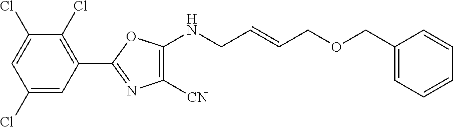 [SMILES: N#Cc1nc(-c2cc(Cl)cc(Cl)c2Cl)oc1NC/C=C/COCc1ccccc1] | 0.23 | 98 |
| 48 | 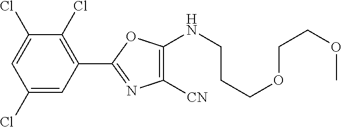 [SMILES: COCCOCCCNc1oc(-c2cc(Cl)cc(Cl)c2Cl)nc1C#N] | 0.62 | 100 |
| 49 | 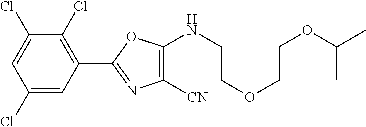 [SMILES: CC(C)OCCOCCNc1oc(-c2cc(Cl)cc(Cl)c2Cl)nc1C#N] | 0.66 | 95 |
| 50 | 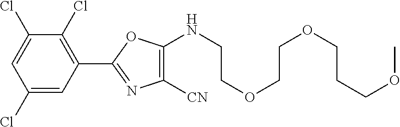 [SMILES: COCCCOCCOCCNc1oc(-c2cc(Cl)cc(Cl)c2Cl)nc1C#N] | 0.89 | 99 |
| 51 | 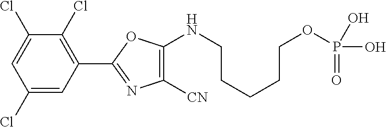 [SMILES: N#Cc1nc(-c2cc(Cl)cc(Cl)c2Cl)oc1NCCCCCOP(=O)(O)O] | >20 | n/a |
| 52 | 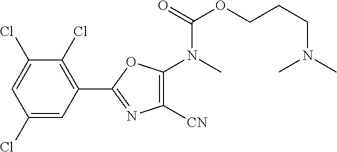 [SMILES: CN(C)CCCOC(=O)N(C)c1oc(-c2cc(Cl)cc(Cl)c2Cl)nc1C#N] | 1.4 | n/a |
| 53 | 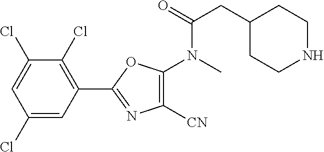 [SMILES: CN(C(=O)CC1CCNCC1)c1oc(-c2cc(Cl)cc(Cl)c2Cl)nc1C#N] | >20 | n/a |
| 54 | 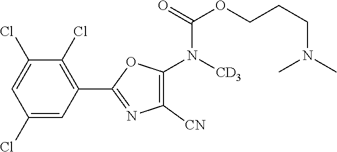 [SMILES: [2H]C([2H])([2H])N(C(=O)OCCCN(C)C)c1oc(-c2cc(Cl)cc(Cl)c2Cl)nc1C#N] | >20 | n/a |
| 55 | 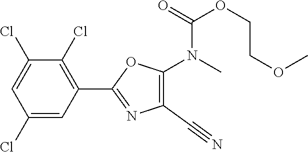 [SMILES: COCCOC(=O)N(C)c1oc(-c2cc(Cl)cc(Cl)c2Cl)nc1C#N] | >20 | n/a |
| 56 | 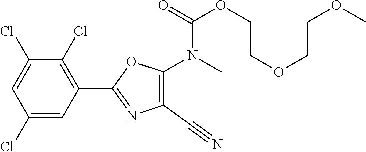 [SMILES: COCCOCCOC(=O)N(C)c1oc(-c2cc(Cl)cc(Cl)c2Cl)nc1C#N] | >20 | n/a |
| 57 | 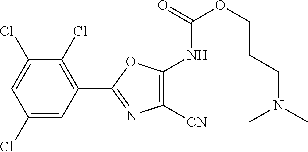 [SMILES: CN(C)CCCOC(=O)Nc1oc(-c2cc(Cl)cc(Cl)c2Cl)nc1C#N] | >20 | n/a |
| 58 | 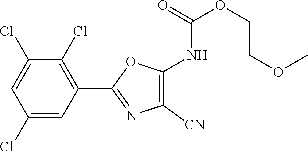 [SMILES: COCCOC(=O)Nc1oc(-c2cc(Cl)cc(Cl)c2Cl)nc1C#N] | >20 | n/a |
| 59 | 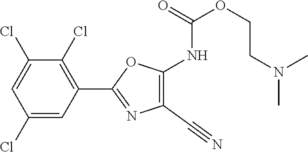 [SMILES: CN(C)CCOC(=O)Nc1oc(-c2cc(Cl)cc(Cl)c2Cl)nc1C#N] | >20 | n/a |
| 60 | 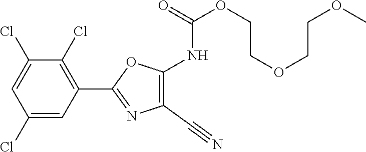 [SMILES: COCCOCCOC(=O)Nc1oc(-c2cc(Cl)cc(Cl)c2Cl)nc1C#N] | >20 | n/a |
| 61 | 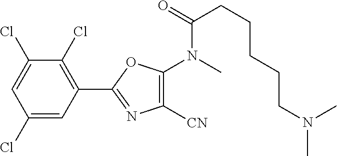 [SMILES: CN(C)CCCCCC(=O)N(C)c1oc(-c2cc(Cl)cc(Cl)c2Cl)nc1C#N] | >20 | n/a |
| 62 | 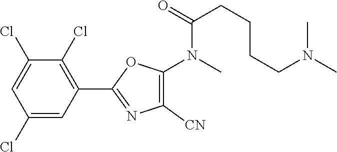 [SMILES: CN(C)CCCCC(=O)N(C)c1oc(-c2cc(Cl)cc(Cl)c2Cl)nc1C#N] | 8.30 | 100 |
| 63 | 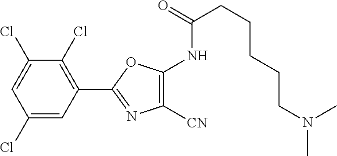 [SMILES: CN(C)CCCCCC(=O)Nc1oc(-c2cc(Cl)cc(Cl)c2Cl)nc1C#N] | >20 | n/a |
| 64 | 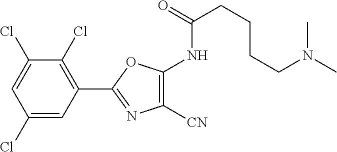 [SMILES: CN(C)CCCCC(=O)Nc1oc(-c2cc(Cl)cc(Cl)c2Cl)nc1C#N] | >20 | n/a |
| 65 | 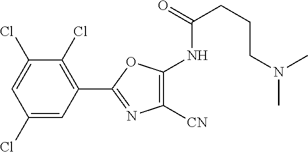 [SMILES: CN(C)CCCC(=O)Nc1oc(-c2cc(Cl)cc(Cl)c2Cl)nc1C#N] | >20 | n/a |
| 66 | 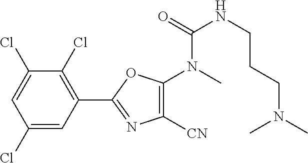 [SMILES: CN(C)CCCNC(=O)N(C)c1oc(-c2cc(Cl)cc(Cl)c2Cl)nc1C#N] | >20 | n/a |
| 67 | 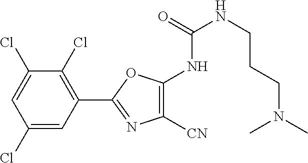 [SMILES: CN(C)CCCNC(=O)Nc1oc(-c2cc(Cl)cc(Cl)c2Cl)nc1C#N] | >20 | n/a |
| 68 | 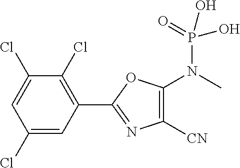 [SMILES: CN(c1oc(-c2cc(Cl)cc(Cl)c2Cl)nc1C#N)P(=O)(O)O] | >20 | n/a |
| 69 | 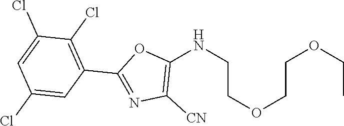 [SMILES: CCOCCOCCNc1oc(-c2cc(Cl)cc(Cl)c2Cl)nc1C#N] | 0.330 | 100 |
| 70 | 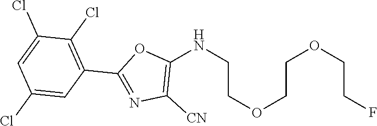 [SMILES: N#Cc1nc(-c2cc(Cl)cc(Cl)c2Cl)oc1NCCOCCOCCF] | 1.10 | 110 |
| 71 | 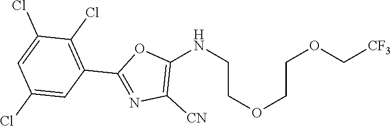 [SMILES: N#Cc1nc(-c2cc(Cl)cc(Cl)c2Cl)oc1NCCOCCOCC(F)(F)F] | 0.160 | 95 |
| 72 | 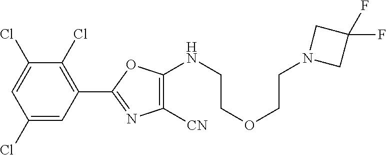 [SMILES: N#Cc1nc(-c2cc(Cl)cc(Cl)c2Cl)oc1NCCOCCN1CC(F)(F)C1] | >20 | n/a |
| 73 | 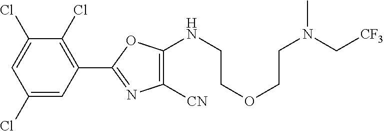 [SMILES: CN(CCOCCNc1oc(-c2cc(Cl)cc(Cl)c2Cl)nc1C#N)CC(F)(F)F] | 0.470 | 70 |
| 74 | 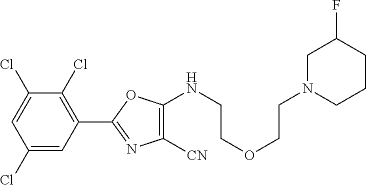 [SMILES: N#Cc1nc(-c2cc(Cl)cc(Cl)c2Cl)oc1NCCOCCN1CCCC(F)C1] | >20 | n/a |
| 75 | 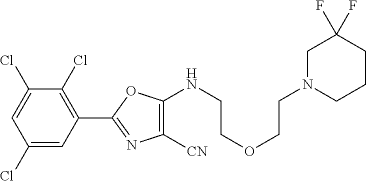 [SMILES: N#Cc1nc(-c2cc(Cl)cc(Cl)c2Cl)oc1NCCOCCN1CCCC(F)(F)C1] | 1.10 | 61 |
| 76 | 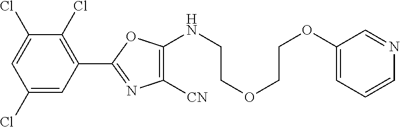 [SMILES: N#Cc1nc(-c2cc(Cl)cc(Cl)c2Cl)oc1NCCOCCOc1cccnc1] | 0.25 | 74 |
| 77 | 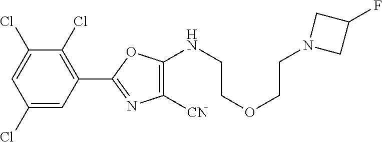 [SMILES: N#Cc1nc(-c2cc(Cl)cc(Cl)c2Cl)oc1NCCOCCN1CC(F)C1] | >20 | n/a |
| 78 | 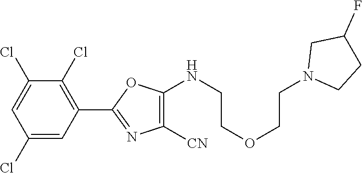 [SMILES: N#Cc1nc(-c2cc(Cl)cc(Cl)c2Cl)oc1NCCOCCN1CCC(F)C1] | >20 | 49 |
| 79 | 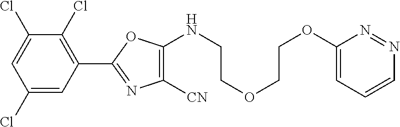 [SMILES: N#Cc1nc(-c2cc(Cl)cc(Cl)c2Cl)oc1NCCOCCOc1cccnn1] | >20 | 17 |
| 80 | 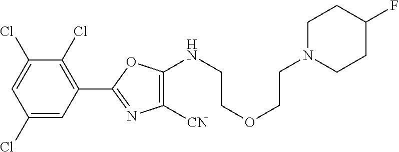 [SMILES: N#Cc1nc(-c2cc(Cl)cc(Cl)c2Cl)oc1NCCOCCN1CCC(F)CC1] | >20 | n/a |
| 81 | 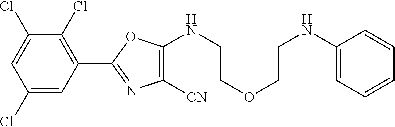 [SMILES: N#Cc1nc(-c2cc(Cl)cc(Cl)c2Cl)oc1NCCOCCNc1ccccc1] | 0.26 | 95 |
| 82 | 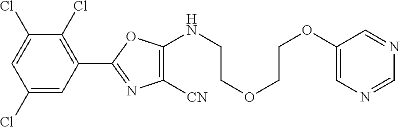 [SMILES: N#Cc1nc(-c2cc(Cl)cc(Cl)c2Cl)oc1NCCOCCOc1cncnc1] | 0.36 | 82 |
| 83 | 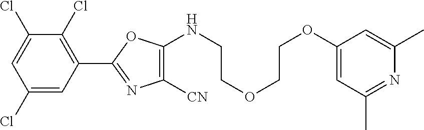 [SMILES: Cc1cc(OCCOCCNc2oc(-c3cc(Cl)cc(Cl)c3Cl)nc2C#N)cc(C)n1] | 0.18 | 61 |
| 84 | 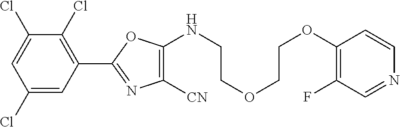 [SMILES: N#Cc1nc(-c2cc(Cl)cc(Cl)c2Cl)oc1NCCOCCOc1ccncc1F] | 0.49 | 68 |
| 85 | 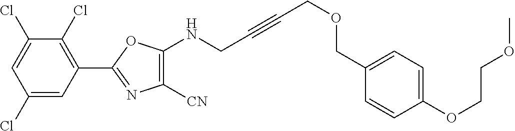 [SMILES: COCCOc1ccc(COCC#CCNc2oc(-c3cc(Cl)cc(Cl)c3Cl)nc2C#N)cc1] | 0.082 | 92 |
| 86 | 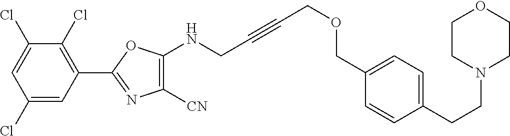 [SMILES: N#Cc1nc(-c2cc(Cl)cc(Cl)c2Cl)oc1NCC#CCOCc1ccc(CCN2CCOCC2)cc1] | 0.23 | 100 |
| 87 | 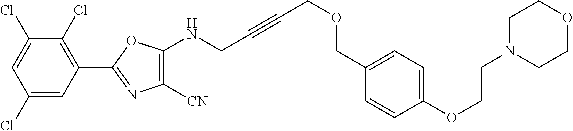 [SMILES: N#Cc1nc(-c2cc(Cl)cc(Cl)c2Cl)oc1NCC#CCOCc1ccc(OCCN2CCOCC2)cc1] | 0.21 | 94 |
| 88 | 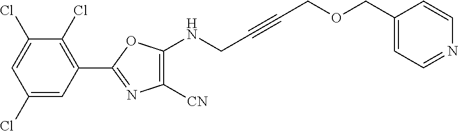 [SMILES: N#Cc1nc(-c2cc(Cl)cc(Cl)c2Cl)oc1NCC#CCOCc1ccncc1] | 0.081 | 74 |
| 89 | 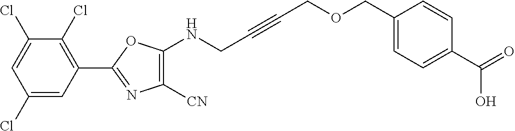 [SMILES: N#Cc1nc(-c2cc(Cl)cc(Cl)c2Cl)oc1NCC#CCOCc1ccc(C(=O)O)cc1] | 1.30 | 100 |
| 90 | 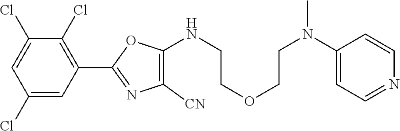 [SMILES: CN(CCOCCNc1oc(-c2cc(Cl)cc(Cl)c2Cl)nc1C#N)c1ccncc1] | >20 | n/a |
| 91 | 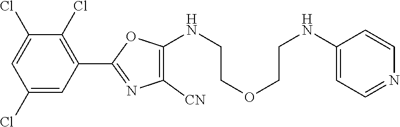 [SMILES: N#Cc1nc(-c2cc(Cl)cc(Cl)c2Cl)oc1NCCOCCNc1ccncc1] | >20 | n/a |
| 92 | 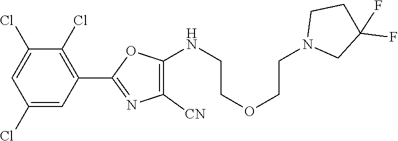 [SMILES: N#Cc1nc(-c2cc(Cl)cc(Cl)c2Cl)oc1NCCOCCN1CCC(F)(F)C1] | 8.80 | 86 |
| 93 | 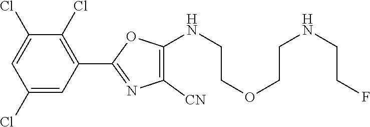 [SMILES: N#Cc1nc(-c2cc(Cl)cc(Cl)c2Cl)oc1NCCOCCNCCF] | 4.90 | 98 |
| 94 | 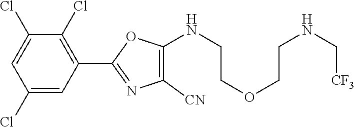 [SMILES: N#Cc1nc(-c2cc(Cl)cc(Cl)c2Cl)oc1NCCOCCNCC(F)(F)F] | 0.30 | 77 |
| 95 | 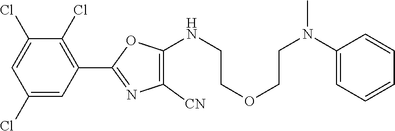 [SMILES: CN(CCOCCNc1oc(-c2cc(Cl)cc(Cl)c2Cl)nc1C#N)c1ccccc1] | 0.26 | 83 |
| 96 | 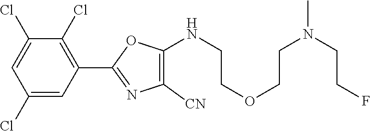 [SMILES: CN(CCF)CCOCCNc1oc(-c2cc(Cl)cc(Cl)c2Cl)nc1C#N] | 4.40 | 93 |
| 97 | 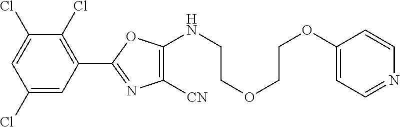 [SMILES: N#Cc1nc(-c2cc(Cl)cc(Cl)c2Cl)oc1NCCOCCOc1ccncc1] | 0.110 | 68 |
| 98 | 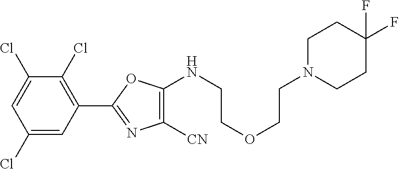 [SMILES: N#Cc1nc(-c2cc(Cl)cc(Cl)c2Cl)oc1NCCOCCN1CCC(F)(F)CC1] | 3.1 | 79 |
| 99 | 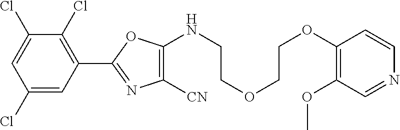 [SMILES: COc1cnccc1OCCOCCNc1oc(-c2cc(Cl)cc(Cl)c2Cl)nc1C#N] | 1.70 | 66 |
| 100 | 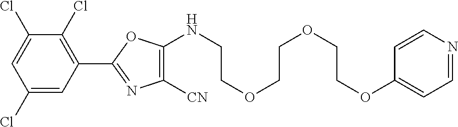 [SMILES: N#Cc1nc(-c2cc(Cl)cc(Cl)c2Cl)oc1NCCOCCOCCOc1ccncc1] | >20 | n/a |
| 101 | 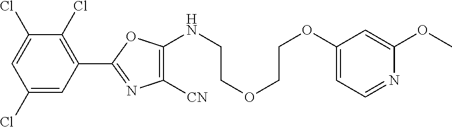 [SMILES: COc1cc(OCCOCCNc2oc(-c3cc(Cl)cc(Cl)c3Cl)nc2C#N)ccn1] | 0.072 | 62 |
| 102 | 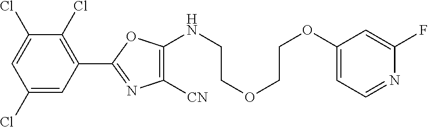 [SMILES: N#Cc1nc(-c2cc(Cl)cc(Cl)c2Cl)oc1NCCOCCOc1ccnc(F)c1] | 0.110 | 86 |
| 103 | 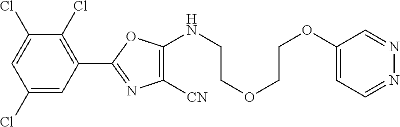 [SMILES: N#Cc1nc(-c2cc(Cl)cc(Cl)c2Cl)oc1NCCOCCOc1ccnnc1] | 4.6 | 73 |

### Example 1A—Preparation of Exemplified Compounds

### Preparation of 3-(dimethylamino) propyl (4-cyano-2-(2,3,5-trichlorophenyl) oxazol-5-yl)(methyl) carbamate hydrochloride (Compound 52)

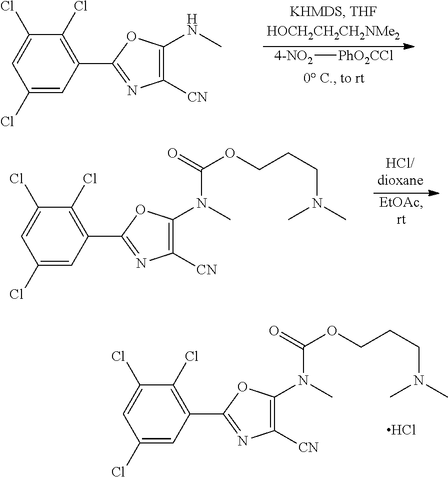

### Step 1. 3-(Dimethylamino) propyl (4-cyano-2-(2,3,5-trichlorophenyl) oxazol-5-yl)(methyl) carbamate

Potassium bis(trimethylsilyl)amide (KHMDS, 1.8 mL, 1.0 M solution in THF, 1.8 mmol, 1.5 equiv) was added drop-wise to a mixture of 5-(methylamino)-2-(2,3,5-trichlorophenyl) oxazole-4-carbonitrile (370 mg, 1.2 mmol) in anhydrous THF (10 mL, 27 volumes of oxazole) at 0° C. under nitrogen, after which the mixture was stirred for 15 min. In a separate flask, potassium bis(trimethylsilyl) amide (KHMDS, 3.1 mL, 1.0 M solution in THF, 3.1 mmol, 2.5 equiv) was added drop-wise to a mixture of 3-dimethylamino-1-propanol (0.36 mL, 3.1 mmol, 2.5 equiv) in anhydrous THF (5 mL, 14 volumes of oxazole) at 0° C. under nitrogen, after which the mixture was stirred for 15 min. To this mixture was added drop-wise a solution of 4-nitrophenyl chloroformate (493 mg, 2.4 mmol, 2.0 equiv) in anhydrous THF (9 mL, 24 volumes of oxazole), after which the mixture was stirred at 0° C. for 30 min. To this mixture was then added drop-wise the KHMDS/oxazole/THF solution, after which the resulting mixture was stirred at 0° C. for 30 min and then slowly warmed to room temperature, stirring for a total of 2 h. The mixture was diluted with saturated aqueous sodium chloride solution (80 mL) and extracted with ethyl acetate (2×80 mL). The combined organic extracts were dried over anhydrous sodium sulfate, filtered and the solvents were removed under reduced pressure. The residue was purified by column chromatography on silica gel, eluting with methanol/ethyl acetate (gradient from 0:100 to 60:40), to provide 3-(dimethylamino) propyl(4-cyano-2-(2,3,5-trichlorophenyl) oxazol-5-yl)(methyl) carbamate (178 mg, 35%) as a colorless oil: ¹H NMR (300 MHz, CDCl₃) δ 7.89 (d, J=2.5 Hz, 1H), 7.63 (d, J=2.5 Hz, 1H), 4.39 (t, J=6.7 Hz, 2H), 3.46 (s, 3H), 2.34 (t, J=7.0 Hz, 2H), 2.22 (s, 6H), 1.98-1.88 (m, 2H) ppm; ESI MS m/z 431 [M+H]⁺.

### Step 2. 3-(Dimethylamino) propyl (4-cyano-2-(2,3,5-trichlorophenyl) oxazol-5-yl)(methyl) carbamate hydrochloride

Hydrochloric acid (1.6 mL, 4.0 N solution in dioxane, 6.4 mmol, 10 equiv.) was added drop-wise to a solution of 3-(dimethylamino) propyl (4-cyano-2-(2,3,5-trichlorophenyl) oxazol-5-yl)(methyl) carbamate (276 mg, 0.64 mmol) in anhydrous ethyl acetate (5.5 mL, 20 volumes of oxazole) at room temperature under nitrogen, after which the mixture was stirred for 10 min. Heptane (15 mL) was added and the resulting mixture was stirred for 5 min, after which the solvents were removed under reduced pressure to produce 3-(dimethylamino) propyl (4-cyano-2-(2,3,5-trichlorophenyl) oxazol-5-yl)(methyl) carbamate hydrochloride (294 mg, 98%) as an off-white solid: ¹H NMR (300 MHz, DMSO-d₆) δ 9.78 (br s, 1H), 8.16 (d, J=2.5 Hz, 1H), 8.06 (d, J=2.5 Hz, 1H), 4.33 (t, J=6.2 Hz, 2H), 3.43 (s, 3H), 3.16 (t, J=5.0 Hz, 2H), 2.76 (s, 6H), 2.14-2.04 (m, 2H) ppm; ESI MS m/z 431 [free base M+H]⁺.

The following compounds were prepared in accordance with the methods and procedures similar to those described above for compound 52

| Cmpd. | | | Mass |
|---|---|---|---|
| No. | Name | NMR | spec |
| 54 | 3-(Dimethylamino)propyl (4- | ¹H NMR (500 MHz, | ESI |
| | cyano-2-(naphthalen-1- | DMSO-d₆) δ 9.98 (s, 1H), | MS m/z |
| | yl)oxazol-5-yl)(methyl- | 8.14 (d, J = 2.5 Hz, 1H), | 434 [M + |
| | d3)carbamate hydrochloride | 8.05 (d, J = 2.5 Hz, 1H), | H]⁺ |
| | | 4.33 (t, J = 6.5 Hz, 2H), | |
| | | 3.16 (t, J = 7.5 Hz, 2H), | |
| | | 2.73 (s, 6H), 2.11 (quint, J = | |
| | | 6.0 Hz, 2H) ppm | |
| 55 | 2-Methoxyethyl (4-cyano-2- | ¹H NMR (300 MHZ, | ESI |
| | (2,3,5-trichlorophenyl)oxazol-5- | CDCl₃) δ 7.88 (d, J = 2.4 | MS m/z |
| | yl)(methyl)carbamate | Hz, 1H), 7.63 (d, J = 2.4 Hz, | 403 [M + |
| | | 1H), 4.49-4.46 (m, 2H), | H]⁺ |
| | | 3.73-3.70 (m, 2H), 3.47 (s, | |
| | | 3H), 3.39 (s, 3H) ppm | |
| 56 | 2-(2-Methoxyethoxy)ethyl (4- | ¹H NMR (300 MHZ, | ESI |
| | cyano-2-(2,3,5- | CDCl₃) δ 7.88 (d, J = 2.4 | MS m/z |
| | trichlorophenyl)oxazol-5- | Hz, 1H), 7.63 (d, J = 2.4 Hz, | 448 [M + |
| | yl)(methyl)carbamate | 1H), 4.50-4.47 (m, 2H), | H]⁺ |
| | | 3.84-3.81 (m, 2H), 3.67- | |
| | | 3.64 (m, 2H), 3.55-3.52 (m, | |
| | | 2H), 3.46 (s, 3H), 3.36 (s, | |
| | | 3H) ppm | |

### Preparation of 3-(dimethylamino) propyl (4-cyano-2-(2,3,5-trichlorophenyl) oxazol-5-yl) carbamate hydrochloride (Compound 57)

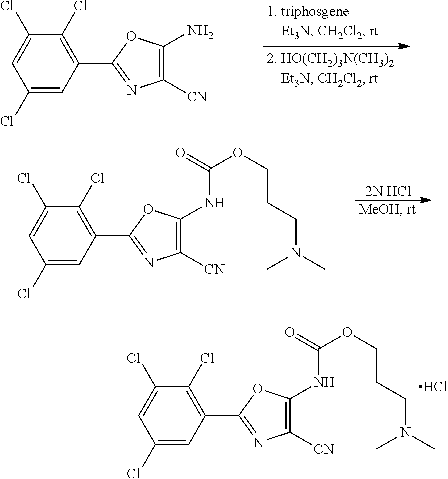 [SMILES: N#Cc1nc(-c2cc(Cl)cc(Cl)c2Cl)oc1N ; CN(C)CCCOC(=O)Nc1oc(-c2cc(Cl)cc(Cl)c2Cl)nc1C#N ; "2N HCl" ; "MeOH, rt" ; CN(C)CCCOC(=O)Nc1oc(-c2cc(Cl)cc(Cl)c2Cl)nc1C#N ; Cl]

### Step 1. 3-(Dimethylamino) propyl (4-cyano-2-(2,3,5-trichlorophenyl) oxazol-5-yl) carbamate

A solution of triphosgene (102 mg, 0.35 mmol) in anhydrous dichloromethane (0.8 mL) was added drop-wise to a solution of 5-amino-2-(2,3,5-trichlorophenyl) oxazole-4-carbonitrile (100 mg, 0.35 mmol) in anhydrous dichloromethane (0.8 mL) at room temperature under nitrogen, after which the resulting suspension was stirred at room temperature for 5 min. A solution of triethylamine (0.10 mL, 0.76 mmol) in anhydrous dichloromethane (0.4 mL) was added drop-wise, and the resulting solution was stirred at room temperature for 3 h. The solvents were removed under reduced pressure and the residue was dissolved in anhydrous dichloromethane (4.0 mL) at room temperature under nitrogen. 3-Dimethylaminopropan-1-ol (60 μL, 0.52 mmol) was added, followed by triethylamine (96 μL, 0.69 mmol), and the mixture was stirred at room temperature for 18 h. The mixture was diluted with water (20 mL) and extracted with dichloromethane (3×20 mL). The combined organic extracts were washed with saturated aqueous sodium chloride solution (2×20 mL) and the solvents were removed under reduced pressure. The residue was purified by column chromatography on silica gel, eluting with methanol/dichloromethane (gradient from 0:100 to 10:90), followed by a second purification by column chromatography on silica gel, eluting with methanol/dichloromethane (gradient from 2:98 to 15:85), to provide 3-(dimethylamino) propyl (4-cyano-2-(2,3,5-trichlorophenyl) oxazol-5-yl) carbamate (17 mg, 11%) an off-white solid: ¹H NMR (300 MHz, DMSO-d₆) δ 7.95 (d, J=2.4 Hz, 1H), 7.80 (d, J=2.4 Hz, 1H), 4.04 (t, J=6.3 Hz, 2H), 3.11-3.03 (m, 2H), 2.74 (s, 6H), 2.11-1.90 (m, 2H) ppm; ESI MS m/z 417 [M+H]⁺.

### Step 2. 3-(Dimethylamino) propyl (4-cyano-2-(2,3,5-trichlorophenyl) oxazol-5-yl) carbamate hydrochloride

2N Hydrochloric acid (0.10 mL, 0.40 mmol), was added to a solution of 3-(dimethylamino) propyl (4-cyano-2-(2,3,5-trichlorophenyl) oxazol-5-yl) carbamate (17 mg, 0.041 mmol) in methanol (1 mL) at room temperature under nitrogen, after which the solvents were removed under reduced pressure. The residue was re-dissolved in anhydrous acetonitrile (2 mL) and the solvents were removed under reduced pressure and lyophilized to provide 3-(dimethylamino) propyl (4-cyano-2-(2,3,5-trichlorophenyl) oxazol-5-yl) carbamate hydrochloride (11 mg, 61%) as a light yellow solid: ¹H NMR (500 MHZ, DMSO-d₆) δ 12.07 (s, 1H), 9.57 (s, 1H), 8.13 (d, J=2.5 Hz, 1H), 7.90 (d, J=2.5 Hz, 1H), 4.28 (t, J=6.3 Hz, 2H), 3.19-3.11 (m, 2H), 2.79 (s, 6H), 2.11-2.01 (m, 2H) ppm; ESI MS m/z 417 [M+H]⁺.

The following compounds were prepared according to methods and procedures similar to those described above for Compound 57

| Cmpd. | | | Mass |
|---|---|---|---|
| No. | Name | NMR | spec |
| 58 | 2-Methoxyethyl (4- | ¹H NMR (500 MHz, DMSO-d₆) δ | ESI MS |
| | cyano-2-(2,3,5- | 12.10 (s, 1H), 8.12 (d, J = 2.5 Hz, | m/z 389 |
| | trichlorophenyl)oxazol-5- | 1H), 7.89 (d, J = 2.5 Hz, 1H), | [M + H]⁺ |
| | yl)carbamate | 4.35-4.31 (m, 2H), 2.62-3.58 (m, | |
| | | 2H), 3.29 (s, 3H) ppm | |
| 59 | 2-(Dimethylamino)ethyl | ¹H NMR (300 MHZ, CDCl₃) δ | ESI MS |
| | (4-cyano-2-(2,3,5- | 11.99 (s, 1H), 10.65 (s, 1H), 7.82 | m/z 401 |
| | trichlorophenyl)oxazol-5- | (d, J = 2.5 Hz, 1H), 7.59 (d, J = | [M − H]⁻ |
| | yl)carbamate | 1.8 Hz, 1H), 4.71-4.64 (m, 2H), | |
| | hydrochloride | 3.44-3.43 (m, 2H), 3.22-2.84 (m, | |
| | | 6H) ppm | |
| 60 | 2-(2- | ¹H NMR (300 MHZ, CDCl₃) δ | ESI MS |
| | Methoxyethoxy)ethyl (4- | 7.85 (d, J = 2.4 Hz, 1H), 7.71 (s, | m/z 432 |
| | cyano-2-(2,3,5- | 1H), 7.62 (d, J = 2.4 Hz, 1H), | [M − H]⁻ |
| | trichlorophenyl)oxazol-5- | 4.49-4.45 (m, 2H), 3.81-3.78 (m, | |
| | yl)carbamate | 2H), 3.69-3.66 (m, 2H), 3.59-3.56 | |
| | | (m, 2H), 3.39 (s, 3H) ppm | |

### Preparation of N-(4-cyano-2-(2,3,5-trichlorophenyl) oxazol-5-yl)-6-(dimethylamino)-N-methylhexanamide hydrochloride (Compound 61)

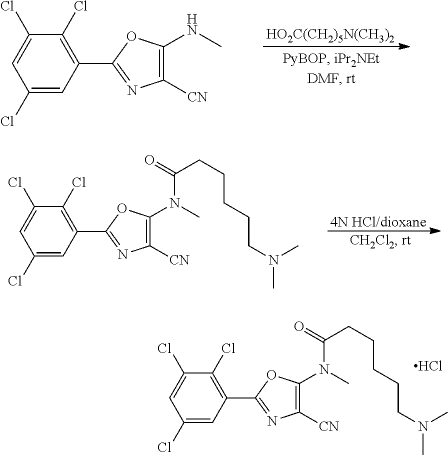

### Step 1. N-(4-Cyano-2-(2,3,5-trichlorophenyl) oxazol-5-yl)-6-(dimethylamino) hexanamide

PyBOP (134 mg, 0.25 mmol) was added to a solution of 6-(dimethylamino) hexanoic acid (39 mg, 0.25 mmol) in anhydrous DMF (1.6 mL) at room temperature under nitrogen, after which 5-(methylamino)-2-(2,3,5-trichlorophenyl) oxazole-4-carbonitrile (50 mg, 0.16 mmol) and diisopropylethylamine (71 μL, 0.41 mmol) were added and the resulting mixture was stirred at room temperature for 18 h. The mixture was diluted with water (50 mL) and extracted with ethyl acetate (3×30 mL). The combined organic extracts were washed with a saturated aqueous sodium bicarbonate solution (2× 30 mL) and saturated aqueous sodium chloride solution (3×20 mL) and the solvents were removed under reduced pressure. The residue was purified by column chromatography on silica gel, eluting with (2:18:80 concentrated ammonium hydroxide/methanol/dichloromethane)/dichloromethane (gradient from 0:100 to 100:0), followed by a second purification by column chromatography on silica gel, eluting with (2:18:80 concentrated ammonium hydroxide/methanol/dichloromethane)/dichloromethane (gradient from 5:95 to 40:60), to provide N-(4-cyano-2-(2,3,5-trichlorophenyl) oxazol-5-yl)-6-(dimethylamino)-N-methylhexanamide (39 mg, 53%) a yellow solid: ¹H NMR (500 MHZ, CDCl₃) δ 7.90 (d, J=2.5 Hz, 1H), 7.66 (d, J=2.5 Hz, 1H), 3.41 (s, 3H), 2.43 (t, J=7.4 Hz, 2H), 2.24 (t, J=7.4 Hz, 2H), 2.20 (s, 6H), 1.76-1.68 (m, 2H), 1.52-1.43 (m, 2H), 1.38-1.30 (m, 2H) ppm.

### Step 2. N-(4-Cyano-2-(2,3,5-trichlorophenyl) oxazol-5-yl)-6-(dimethylamino)-N-methylhexanamide hydrochloride

Hydrochloric acid (24 μL, 0.096 mmol, 4N solution in dioxane), was added to a solution of N-(4-cyano-2-(2,3,5-trichlorophenyl) oxazol-5-yl)-6-(dimethylamino)-N-methylhexanamide (39 mg, 0.088 mmol) in anhydrous dichloromethane (2 mL) at room temperature under nitrogen, after which the mixture was stirred at room temperature for 2 h. The resulting suspension was triturated with diethyl ether (20 mL). The solids were collected by filtration under reduced pressure to provide N-(4-cyano-2-(2,3,5-trichlorophenyl) oxazol-5-yl)-6-(dimethylamino)-N-methylhexanamide hydrochloride (36 mg, 86%) as an off-white solid: ¹H NMR (500 MHZ, DMSO-d₆) δ 9.85 (s, 1H), 8.15 (d, J=2.5 Hz, 1H), 8.06 (d, J=2.5 Hz, 1H), 3.45 (s, 3H), 3.04-2.97 (m, 2H), 2.73 (s, 6H), 2.64 (t, J=7.2 Hz, 2H), 1.71 (m, 4H), 1.38-1.28 (m, 2H) ppm; ESI MS m/z 443 [M+H]⁺.

The following compounds were prepared according to methods and procedures similar to those described above for Compound 61

| Cmpd. | | | Mass |
|---|---|---|---|
| No. | Name | NMR | spec |
| 62 | N-(4-Cyano-2-(2,3,5- | ¹H NMR (500 MHz, CDCl₃) δ | ESI MS |
| | trichlorophenyl)oxazol-5- | 12.59 (s, 1H), 7.92 (d, J = 2.5 Hz, | m/z 429 |
| | yl)-5-(dimethylamino)- | 1H), 7.67 (d, J = 2.5 Hz, 1H), 3.44 | [M + H]⁺ |
| | N-methylpentanamide | (s, 3H), 3.04-2.97 (m, 2H), 2.82- | |
| | hydrochloride | 2.78 (m, 6H), 2.60-2.52 (m, 2H), | |
| | | 2.02-1.92 (m, 2H), 1.86-1.76 (m, | |
| | | 2H) ppm | |

### Preparation of N-(4-cyano-2-(2,3,5-trichlorophenyl) oxazol-5-yl)-6-(dimethylamino) hexanamide hydrochloride (Compound 63)

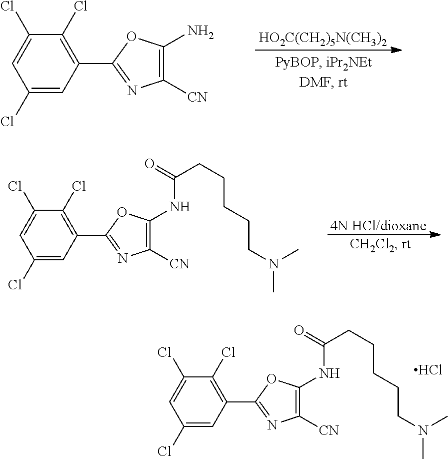

### Step 1. N-(4-Cyano-2-(2,3,5-trichlorophenyl) oxazol-5-yl)-6-(dimethylamino) hexanamide

PyBOP (135 mg, 0.26 mmol) was added to a solution of 6-(dimethylamino) hexanoic acid (41 mg, 0.26 mmol) in anhydrous DMF (1.8 mL) at room temperature under nitrogen, after which 5-amino-2-(2,3,5-trichlorophenyl) oxazole-4-carbonitrile (50 mg, 0.17 mmol) and diisopropylethylamine (75 μL, 0.43 mmol) were added and the resulting mixture was stirred at room temperature for 18 h. The mixture was diluted with water (50 mL) and extracted with ethyl acetate (3×30 mL). The combined organic extracts were washed with a saturated aqueous sodium bicarbonate solution (2×30 mL) and saturated aqueous sodium chloride solution (3×20 mL) and the solvents were removed under reduced pressure. The residue was purified by column chromatography on silica gel, eluting with (2:18:80 concentrated ammonium hydroxide/methanol/dichloromethane)/dichloromethane (gradient from 0:100 to 100:0), to provide N-(4-cyano-2-(2,3,5-trichlorophenyl) oxazol-5-yl)-6-(dimethylamino) hexanamide (59 mg, 79%) a yellow solid: ¹H NMR (500 MHZ, CDCl₃) δ 7.87 (d, J=2.5 Hz, 1H), 7.55 (d, J=2.4 Hz, 1H), 2.83 (t, J=6.5 Hz, 2H), 2.64 (s, 6H), 2.58-2.54 (m, 2H), 1.82-1.71 (m, 4H), 1.62-1.55 (m, 2H) ppm.

### Step 2. N-(4-Cyano-2-(2,3,5-trichlorophenyl) oxazol-5-yl)-6-(dimethylamino) hexanamide hydrochloride

Hydrochloric acid (34 μL, 0.14 mmol, 4N solution in dioxane), was added to a solution of N-(4-cyano-2-(2,3,5-trichlorophenyl) oxazol-5-yl)-6-(dimethylamino) hexanamide (59 mg, 0.14 mmol) in anhydrous dichloromethane (2 mL) at room temperature under nitrogen, after which the resulting suspension was triturated with diethyl ether (10 mL). The solids were collected by filtration under reduced pressure to provide N-(4-cyano-2-(2,3,5-trichlorophenyl) oxazol-5-yl)-6-(dimethylamino) hexanamide hydrochloride (49 mg, 83%) as an off-white solid: 1H NMR (500 MHZ, MeOD-d₄) δ 7.92 (d, J=2.5 Hz, 1H), 7.86 (d, J=2.5 Hz, 1H), 3.19-3.11 (m, 2H), 2.89 (s, 6H), 2.56 (t, J=7.1 Hz, 2H), 1.83-1.72 (m, 4H), 1.53-1.43 (m, 2H) ppm; ESI MS m/z 429 [M+H]⁺.

The following compounds were prepared according to methods and procedures similar to those described above for Compound 63

| Cmpd. | | | |
|---|---|---|---|
| No. | Name | NMR | Mass spec |
| 64 | N-(4-Cyano-2-(2,3,5- | ¹H NMR (500 MHz, | ESI MS m/z |
| | trichlorophenyl)oxazol-5-yl)- | CDCl₃) δ 11.56 (br s, 1H), | 415 [M + H]⁺ |
| | 5- | 11.37 (br s, 1H), 7.84 (d, J = | |
| | (dimethylamino)pentanamide | 2.5 Hz, 1H), 7.57 (d, J = | |
| | hydrochloride | 2.5 Hz, 1H), 3.19-3.13 (m, | |
| | | 2H), 2.88 (s, 6H), 2.72 (t, J = | |
| | | 7.0 Hz, 2H), 2.05-1.93 | |
| | | (m, 4H) ppm | |
| 65 | N-(4-Cyano-2-(2,3,5- | ¹H NMR (500 MHz, | ESI MS m/z |
| | trichlorophenyl)oxazol-5-yl)- | DMSO-d₆) δ 12.30 (s, 1H), | 399 [M − H]⁻ |
| | 4- | 9.79 (s, 1H), 8.13 (d, J = | |
| | (dimethylamino)butanamide | 2.5 Hz, 1H), 7.92 (d, J = | |
| | hydrochloride | 2.5 Hz, 1H), 3.11-3.07 (m, | |
| | | 2H), 2.78 (s, 6H), 2.59 (t, J = | |
| | | 7.2 Hz, 2H), 2.00-1.93 | |
| | | (m, 2H) ppm | |

### Preparation of 1-(4-cyano-2-(2,3,5-trichlorophenyl) oxazol-5-yl)-3-(3-(dimethylamino) propyl)-1-methylurea hydrochloride (Compound 66)

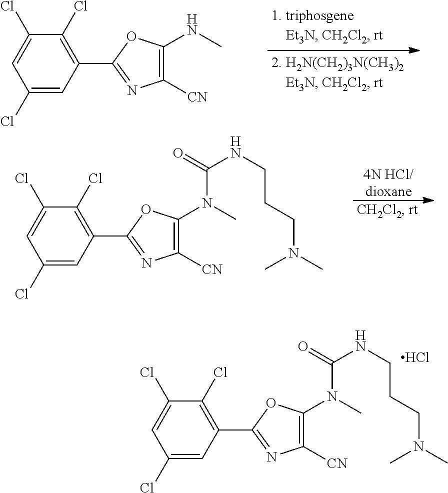 [SMILES: CNc1oc(-c2cc(Cl)cc(Cl)c2Cl)nc1C#N ; CN(C)CCCNC(=O)N(C)c1oc(-c2cc(Cl)cc(Cl)c2Cl)nc1C#N ; "CH2Cl2, rt" ; "4N HCl/dioxane" ; CN(C)CCCNC(=O)N(C)c1oc(-c2cc(Cl)cc(Cl)c2Cl)nc1C#N ; Cl]

### Step 1. 1-(4-Cyano-2-(2,3,5-trichlorophenyl) oxazol-5-yl)-3-(3-(dimethylamino) propyl)-1-methylurea

A solution of triphosgene (983 mg, 0.33 mmol) in anhydrous dichloromethane (0.6 mL) was added drop-wise to a solution of 5-(methylamino)-2-(2,3,5-trichlorophenyl) oxazole-4-carbonitrile (100 mg, 0.33 mmol) in anhydrous dichloromethane (0.6 mL) at room temperature under nitrogen, after which the resulting suspension was stirred at room temperature for 5 min. A solution of triethylamine (0.10 mL, 0.73 mmol) in anhydrous dichloromethane (0.3 mL) was added drop-wise, and the resulting solution was stirred at room temperature for 1.5 h. The solvents were removed under reduced pressure and the residue was dissolved in anhydrous dichloromethane (4.0 mL) at room temperature under nitrogen. 3-Dimethylaminopropylamine (83 μL, 0.66 mmol) was added, followed by triethylamine (91 μL, 0.66 mmol), and the mixture was stirred at room temperature for 2 h. The mixture was diluted with water (20 mL) and extracted with dichloromethane (3×20 mL). The combined organic extracts were washed with saturated aqueous sodium chloride solution (2× 20 mL) and the solvents were removed under reduced pressure. The residue was purified by column chromatography on silica gel, eluting with (2:18:80 concentrated ammonium hydroxide/methanol/dichloromethane)/dichloromethane (gradient from 25:75 to 75:25), followed by a second purification by column chromatography on silica gel, eluting with (2:18:80 concentrated ammonium hydroxide/methanol/dichloromethane)/dichloromethane (gradient from 0:100 to 20:80), to provide 1-(4-cyano-2-(2,3,5-trichlorophenyl) oxazol-5-yl)-3-(3-(dimethylamino) propyl)-1-methylurea (26 mg, 18%) a yellow solid: ¹H NMR (300 MHz, DMSO-d₆) δ 8.11-8.08 (m, 2H), 7.76 (s, 1H), 4.09-3.98 (m, 2H), 3.43 (s, 3H), 2.27 (t, J=3.6 Hz, 2H), 2.12 (s, 6H), 1.77-1.66 (m, 2H) ppm.

### Step 2. 1-(4-Cyano-2-(2,3,5-trichlorophenyl) oxazol-5-yl)-3-(3-(dimethylamino) propyl)-1-methylurea hydrochloride

Hydrochloric acid (16 μL, 0.066 mmol, 4N solution in dioxane), was added to a solution of 1-(4-cyano-2-(2,3,5-trichlorophenyl) oxazol-5-yl)-3-(3-(dimethylamino) propyl)-1-methylurea (26 mg, 0.060 mmol) in anhydrous dichloromethane (2 mL) at room temperature under nitrogen, after which the resulting suspension was triturated with diethyl ether (20 mL). The solids were collected by filtration under reduced pressure to provide 1-(4-cyano-2-(2,3,5-trichlorophenyl) oxazol-5-yl)-3-(3-(dimethylamino) propyl)-1-methylurea hydrochloride (25 mg, 83%) as an off-white solid: ¹H NMR (500 MHz, DMSO-d₆) δ 10.63 (br s, 1H), 10.21 (br s, 2H), 8.27-8.16 (m, 2H), 4.15 (t, J=7.1 Hz, 2H), 3.63 (s, 3H), 3.21-3.09 (m, 2H), 2.76 (s, 6H), 2.06-1.95 (m, 2H) ppm; ESI MS m/z 430 [M+H]⁺.

### Preparation of 1-(4-cyano-2-(2,3,5-trichlorophenyl) oxazol-5-yl)-3-(3-(dimethylamino) propyl) urea hydrochloride (Compound 67)

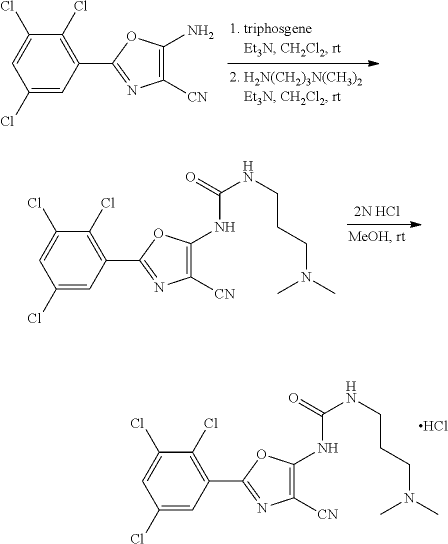

### Step 1. 1-(4-Cyano-2-(2,3,5-trichlorophenyl) oxazol-5-yl)-3-(3-(dimethylamino) propyl) urea

A solution of triphosgene (103 mg, 0.35 mmol) in anhydrous dichloromethane (0.9 mL) was added drop-wise to a solution of 5-amino-2-(2,3,5-trichlorophenyl) oxazole-4-carbonitrile (100 mg, 0.35 mmol) in anhydrous dichloromethane (0.9 mL) at room temperature under nitrogen, after which the resulting suspension was stirred at room temperature for 5 min. A solution of triethylamine (0.11 mL, 0.76 mmol) in anhydrous dichloromethane (0.5 mL) was added drop-wise, and the resulting solution was stirred at room temperature for 3 h. The solvents were removed under reduced pressure and the residue was dissolved in anhydrous dichloromethane (4.0 mL) at room temperature under nitrogen. 3-Dimethylaminopropylamine (65 μL, 0.52 mmol) was added, followed by triethylamine (95 μL, 0.69 mmol), and the mixture was stirred at room temperature for 18 h. The mixture was diluted with water (20 mL) and extracted with dichloromethane (3×20 mL). The combined organic extracts were washed with saturated aqueous sodium chloride solution (2×20 mL) and the solvents were removed under reduced pressure. The residue was purified by column chromatography on silica gel, eluting with (2:18:80 concentrated ammonium hydroxide/methanol/dichloromethane)/dichloromethane (gradient from 0:100 to 50:50), followed by a second purification by column chromatography on silica gel, eluting with (2:18:80 concentrated ammonium hydroxide/methanol/dichloromethane)/dichloromethane (gradient from 0:100 to 50:50), to provide 1-(4-cyano-2-(2,3,5-trichlorophenyl) oxazol-5-yl)-3-(3-(dimethylamino) propyl) urea (33 mg, 23%) a yellow solid: ¹H NMR (300 MHZ, DMSO-d₆) δ 8.94 (br s, 2H), 8.09 (d, J=2.5 Hz, 1H), 8.00 (d, J=2.5 Hz, 1H), 3.97-3.85 (m, 2H), 2.31-2.22 (m, 2H), 2.18 (s, 6H), 1.82-1.71 (m, 2H) ppm.

### Step 2. 1-(4-Cyano-2-(2,3,5-trichlorophenyl) oxazol-5-yl)-3-(3-(dimethylamino) propyl) urea hydrochloride

2N Hydrochloric acid (45 μL, 0.090 mmol), was added to a solution of 1-(4-cyano-2-(2,3,5-trichlorophenyl) oxazol-5-yl)-3-(3-(dimethylamino) propyl) urea (33 mg, 0.080 mmol) in methanol (2 mL) at room temperature under nitrogen, after which the solvents were removed under reduced pressure. The residue was re-dissolved in anhydrous acetonitrile (2 mL) and the solvents were removed under reduced pressure and lyophilized to provide 3-(dimethylamino) propyl (4-cyano-2-(2,3,5-trichlorophenyl) oxazol-5-yl) carbamate hydrochloride (30 mg, 82%) as a light yellow solid: ¹H NMR (500 MHZ, DMSO-d₆) δ 9.83 (br s, 1H), 8.75 (br s, 2H), 8.09 (d, J=2.5 Hz, 1H), 7.99 (d, J=2.5 Hz, 1H), 4.03 (t, J=7.1 Hz, 2H), 3.15-3.07 (m, 2H), 2.78-2.74 (m, 6H), 2.00-1.90 (m, 2H) ppm; ESI MS m/z 416 [M+H]⁺.

### Preparation of (4-cyano-2-(2,3,5-trichlorophenyl) oxazol-5-yl)(methyl)phosphoramidic acid (Compound 68)

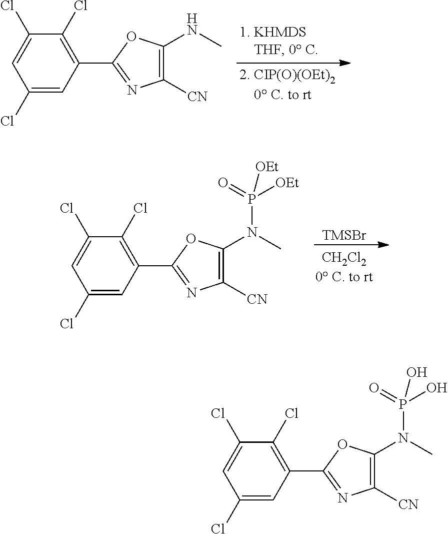 [SMILES: "TMSBr" ; CNc1oc(-c2cc(Cl)cc(Cl)c2Cl)nc1C#N ; CCOP(=O)(OCC)N(C)c1oc(-c2cc(Cl)cc(Cl)c2Cl)nc1C#N ; CN(c1oc(-c2cc(Cl)cc(Cl)c2Cl)nc1C#N)P(=O)(O)O]

### Step 1. Diethyl (4-cyano-2-(2,3,5-trichlorophenyl) oxazol-5-yl)(methyl)phosphoramidate

Potassium bis(trimethylsilyl) amide (KHMDS, 0.94 mL, 0.94 mmol, 1.0 M solution in THF, 1.5 equiv) was added drop-wise to a solution of 5-(methylamino)-2-(2,3,5-trichlorophenyl) oxazole-4-carbonitrile (190 mg, 0.63 mmol) in anhydrous THF (5.7 mL, 30 volumes of oxazole) at 0° C. under nitrogen, after which the mixture was stirred for 10 min. Diethyl chlorophosphate (0.27 mL, 1.9 mmol, 3.0 equiv) was added drop-wise, after which the mixture was slowly warmed to room temperature, stirring for a total of 2 h. The mixture was poured into saturated aqueous sodium chloride solution (30 mL) and extracted with ethyl acetate (2×30 mL). The combined organic extracts were dried over anhydrous sodium sulfate, filtered and the solvents were removed under reduced pressure. The residue was purified by column chromatography on silica gel, eluting with ethyl acetate/heptane (gradient from 0:100 to 50:50), to provide diethyl (4-cyano-2-(2,3,5-trichlorophenyl) oxazol-5-yl)(methyl)phosphoramidate (190 mg, 69%) as a tan solid: ¹H NMR (300 MHZ, CDCl₃) δ 7.85 (d, J=2.5 Hz, 1H), 7.61 (d, J=2.6 Hz, 1H), 4.35-4.14 (m, 4H), 3.41 (d, J=7.9 Hz, 3H), 1.63 (br s, 1H), 3.40 (t, J=7.02 Hz, 6H) ppm.

### Step 2. (4-Cyano-2-(2,3,5-trichlorophenyl) oxazol-5-yl)(methyl)phosphoramidic acid

Bromotrimethylsilane (0.55 mL, 4.3 mmol, 10.0 equiv) was added drop-wise to a solution of diethyl (4-cyano-2-(2,3,5-trichlorophenyl) oxazol-5-yl)(methyl)phosphoramidate (190 mg, 0.43 mmol) in anhydrous dichloromethane (1.9 mL, 10 volumes of phosphoramidate) at 0° C. under nitrogen, after which the mixture was slowly warmed to room temperature, stirring for a total of 17 h. The solvent was removed under reduced pressure and the residue was dissolved in THF (5 mL), treated with water (1 mL) and the biphasic mixture was stirred at room temperature for 30 min. The solvents were removed under reduced pressure and the residue was purified by reverse phase column chromatography, eluting with acetonitrile/water (gradient from 0:100 to 100:0), to produce (4-cyano-2-(2,3,5-trichlorophenyl) oxazol-5-yl)(methyl)phosphoramidic acid (71 mg, 43%) as an off-white solid: ¹H NMR (500 MHz, DMSO-d₆) δ 7.95 (d, J=2.5 Hz, 1H), 7.91 (d, J=2.2 Hz, 1H), 7.28 (br s, 1H), 6.98 (br s, 2H), 2.98 (d, J=4.9 Hz, 3H) ppm.

### Preparation of 5-((4-cyano-2-(2,3,5-trichlorophenyl) oxazol-5-yl)amino) pentyl dihydrogen phosphate (Compound 51)

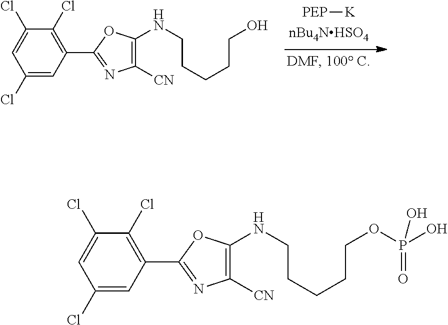

Anhydrous DMF (4.1 mL, 21 volumes of oxazole) was charged to a flask containing 5-((5-hydroxypentyl)-amino)-2-(2,3,5-trichlorophenyl) oxazole-4-carbonitrile (200 mg, 0.54 mmol), tetrabutylammonium bisulfate (109 mg, 0.32 mmol, 0.60 equiv) and phosphoenolpyruvic acid monopotassium salt (PEP-K, 646 mg, 3.2 mmol, 6.0 equiv) at room temperature under nitrogen, after which the mixture was heated to 100° C. to stir for 6 h. The mixture was cooled to room temperature and directly purified by reverse phase column chromatography, eluting with acetonitrile/water (gradient from 5:95 to 100:0), to produce 5-((4-cyano-2-(2,3,5-trichlorophenyl) oxazol-5-yl)amino) pentyl dihydrogen phosphate (154 mg, 63%) as an off-white solid: ¹H NMR (500 MHz, DMSO-d₆) δ 8.74 (t, J=5.8 Hz, 1H), 7.97 (d, J=2.3 Hz, 1H), 7.84 (d, J=2.8 Hz, 1H), 3.80 (q, J=6.8 Hz, 2H), 3.35 (q, J=6.2 Hz, 2H), 1.65-1.57 (m, 4H), 1.44-1.38 (m, 2H) ppm; ESI MS m/z 454 [M+H]⁺.

### Preparation of 5-((methyl-d3)amino)-2-(2,3,5-trichlorophenyl) oxazole-4-carbonitrile (Compound 44)

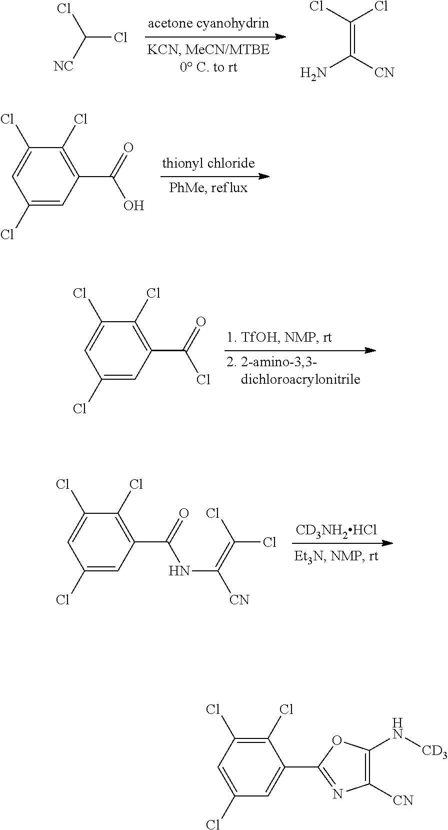

### Step 1. 2-Amino-3,3-dichloroacrylonitrile

Dichloroacetonitrile (14.6 g, 181.9 mmol) was added to a mixture of anhydrous methyl tert-butyl ether (180 mL) and anhydrous acetonitrile (720 mL) at 0° C. under nitrogen in a 2 L flask that was wrapped in foil to exclude light. Acetone cyanohydrin (18.3 mL, 200.1 mmol) was added followed by potassium cyanide (236 mg, 3.64 mmol) and the resulting mixture was slowly warmed to room temperature, stirring for a total of 18 h. The solvents were removed under reduced pressure and the residue was partitioned between methyl tert-butyl ether (150 mL) and saturated aqueous sodium bicarbonate solution (150 mL) in a flask covered to exclude light and the biphasic mixture was stirred at room temperature under the exclusion of light for 2 h. The organic phase was collected and washed with saturated aqueous sodium bicarbonate solution (3×100 mL). The aqueous washings were then back-extracted with methyl tert-butyl ether (2×100 mL). The combined organic extracts were washed with saturated aqueous sodium chloride solution (100 mL), treated with anhydrous sodium sulfate and activated carbon (6 g) and the resulting mixture was stirred at room temperature under the exclusion of light for 30 min. The mixture was filtered through Celite under reduced pressure and the filtrate solvents were removed under reduced pressure. The dried residue was again partitioned between methyl tert-butyl ether (100 mL) and saturated aqueous sodium bicarbonate solution (100 mL) in a flask covered to exclude light and the biphasic mixture was stirred at room temperature under the exclusion of light for 2.5 h. The organic phase was collected and washed with saturated aqueous sodium bicarbonate solution (4×75 mL). The aqueous washings were then back-extracted with methyl tert-butyl ether (3×75 mL). The combined organic extracts were washed with saturated aqueous sodium chloride solution (100 mL), treated with anhydrous sodium sulfate and activated carbon (6 g) and the resulting mixture was stirred at room temperature under the exclusion of light for 20 min. The mixture was filtered through Celite under reduced pressure and the filtrate solvents were removed under reduced pressure under the exclusion of light to provide 2-amino-3,3-dichloroacrylonitrile (15.3 g, 69%) as a light yellow solid: 1H NMR (500 MHz, CDCl₃) δ 3.66 (br s, 2H) ppm.

### Step 2. 2,3,5-Trichlorobenzoyl chloride

Thionyl chloride (5.27 g, 44.3 mmol) was added portion-wise to a solution of 2,3,5-trichlorobenzoic acid (2.00 g, 8.87 mmol) in anhydrous toluene (48 mL) at room temperature under nitrogen, after which the mixture was heated to reflux to stir for 4.5 h. The mixture was cooled to room temperature and the solvent was removed under reduced pressure. The residue was subjected to three iterations of treatment with toluene (5 mL) followed by removal of the solvent under reduced pressure to produce 2,3,5-trichlorobenzoyl chloride (2.16 g, quantitative) as an orange waxy solid: ¹H NMR (300 MHz, CDCl₃) δ 7.86 (d, J=2.4 H, 1H), 7.69 (d, J=2.4 Hz, 1H) ppm.

### Step 3. 2,3,5-Trichloro-N-(2,2-dichloro-1-cyanovinyl)benzamide

Trifluoromethanesulfonic acid (0.88 M solution in N-methyl pyrrolidinone, 52.9 mL, 46.6 mmol) was slowly added via addition funnel to a solution of 2,3,5-trichlorobenzoyl chloride (10.78 g, 44.4 mmol) in anhydrous N-methyl pyrrolidinone (24 mL) at 0° C. under nitrogen in a flask that was wrapped in foil to exclude light. 2-Amino-3,3-dichloroacrylonitrile (6.68 g, 48.8 mmol) was added portion-wise and the resulting mixture was slowly warmed to room temperature, stirring for a total of 18 h. The mixture was diluted with water (300 mL) and the resulting solids were collected by filtration under reduced pressure, washing with water (100 mL). The solids were dissolved in ethyl acetate (1.5 L) in a flask that was wrapped in foil to exclude light, dried over anhydrous sodium sulfate and filtered. The solvent was removed under reduced pressure to provide 2,3,5-trichloro-N-(2,2-dichloro-1-cyanovinyl)benzamide (14.2 g, 93%) as a yellow solid (14.2 g, 93%): ¹H NMR (300 MHz, DMSO-d₆) δ 11.15 (s, 1H), 8.06 (d, J=2.4 Hz, 1H), 7.78 (d, J=2.4 Hz, 1H) ppm.

### Step 4. 5-((Methyl-d₃)amino)-2-(2,3,5-trichlorophenyl) oxazole-4-carbonitrile

Methan-d₃-amine hydrochloride (0.107 g, 1.52 mmol) was added portion-wise to a solution of 2,3,5-trichloro-N-(2,2-dichloro-1-cyanovinyl)benzamide (350 mg, 1.02 mmol) in anhydrous N-methyl pyrrolidinone (1.10 mL) at room temperature under nitrogen in a flask that was wrapped in foil to exclude light, and the resulting mixture was stirred at room temperature for 18 h. Additional methan-d₃-amine hydrochloride (20.0 mg, 0.284 mmol) was added and the mixture was stirred at room temperature for an additional 4 h. The mixture was diluted with water (100 mL) and the resulting solids were collected by filtration under reduced pressure, washing with water (50 mL). The solids were further purified by column chromatography on silica gel, eluting with ethyl acetate/heptane (gradient from 0:100 to 30:70), to provide 5-((methyl-d₃)amino)-2-(2,3,5-trichlorophenyl) oxazole-4-carbonitrile (110 mg, 34%) as a white solid: ¹H NMR (500 MHz, DMSO-d₆) δ 8.55 (s, 1H), 7.99 (d, J=2.5 Hz, 1H), 7.86 (d, J=2.5 Hz, 1H) ppm; ESI MS m/z 304 [M+H]⁺.

### Preparation of 5-((2-(2-Methoxyethoxy)ethyl)amino)-2-(2,3,5-trichlorophenyl) oxazole-4-carbonitrile (Compound 32)

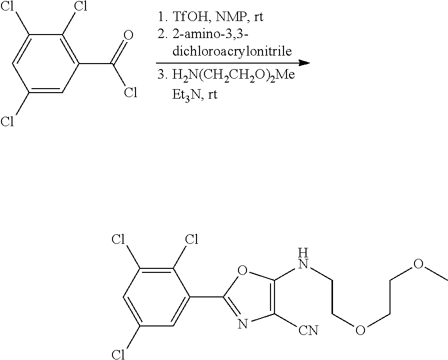 [SMILES: O=C(Cl)c1cc(Cl)cc(Cl)c1Cl ; COCCOCCNc1oc(-c2cc(Cl)cc(Cl)c2Cl)nc1C#N]

Trifluoromethanesulfonic acid (78 μL, 0.887 mmol) was added to a solution of 2,3,5-trichlorobenzoyl chloride (216 mg, 0.887 mmol) in anhydrous N-methylpyrrolidinone (1.0 mL) at room temperature under nitrogen in a flask that was wrapped in foil to exclude light. 2-Amino-3,3-dichloroacrylonitrile (133 mg, 0.976 mmol) was added portion-wise and the resulting mixture was slowly warmed to room temperature, stirring for a total of 18 h. Triethylamine (0.61 mL, 4.43 mmol) was added followed by 2-(2-methoxyethoxy) ethan-1-amine (0.17 mL, 1.33 mmol), and the resulting mixture was stirred at room temperature for 18 h. The mixture was diluted with water (40 mL) and extracted with ethyl acetate (3×30 mL). The combined organic extracts were washed with a saturated aqueous sodium chloride solution (3×20 mL) and the solvents were removed under reduced pressure. The residue was purified by column chromatography on silica gel, eluting with methanol/dichloromethane (gradient from 0:100 to 10:90), to provide 5-((2-(2-methoxyethoxy)ethyl)amino)-2-(2,3,5-trichlorophenyl) oxazole-4-carbonitrile (cmpd. 32, 99 mg, 28%) as a white solid: ¹H NMR (500 MHz, DMSO-d₆) δ 8.76 (t, J=5.8 Hz, 1H), 7.99 (d, J=2.5 Hz, 1H), 7.87 (d, J=2.5 Hz, 1H), 3.62-3.58 (m, 2H), 3.57-3.54 (m, 2H), 3.53-3.49 (m, 2H), 3.44-3.41 (m, 2H) 3.20 (s, 3H) ppm; ESI MS m/z 390 [M+H]⁺.

### Preparation of 5-(methylamino)-2-(2,3,5-trichlorophenyl) oxazole-4-carbonitrile (Compound 1)—synthetic route 1

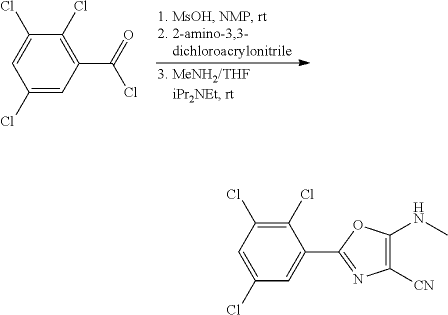 [SMILES: O=C(Cl)c1cc(Cl)cc(Cl)c1Cl ; CNc1oc(-c2cc(Cl)cc(Cl)c2Cl)nc1C#N]

Methanesulfonic acid (1.44 mL, 22.2 mmol mmol) was added drop-wise to a solution of 2,3,5-trichlorobenzoyl chloride (5.43 g, 22.2 mmol) in anhydrous N-methylpyrrolidinone (24 mL) at room temperature under nitrogen in a flask that was wrapped in foil to exclude light. 2-Amino-3,3-dichloroacrylonitrile (3.34 g, 24.4 mmol) was added portion-wise and the resulting mixture was slowly warmed to room temperature, stirring for a total of 18 h. The mixture was cooled to 0° C., after which diisopropylethylamine (19.3 mL, 111 mmol) was added followed by methylamine (22.2 mL, 44.4 mmol, 2.0 M solution in THF), and the resulting mixture was slowly warmed to room temperature, stirring for a total of 18 h. The mixture was diluted with water (500 mL) and the solids were collected by filtration under reduced pressure. The solids were triturated with methanol (50 mL) for 30 min, after which the solids were collected by filtration under reduced pressure to provide 5-(methylamino)-2-(2,3,5-trichlorophenyl) oxazole-4-carbonitrile (5.27 g, 78%) as an off-white solid: ¹H NMR (500 MHZ, DMSO-d₆) δ 8.57 (q, J=4.8 Hz, 1H), 7.99 (d, J=2.5 Hz, 1H), 7.87 (d, J=2.4 Hz, 1H), 3.00 (d, J=4.8 Hz, 3H) ppm; ESI MS m/z 302 [M+H]⁺.

### Preparation of 5-(methylamino)-2-(2,3,5-trichlorophenyl) oxazole-4-carbonitrile (Compound 1)-synthetic route 2

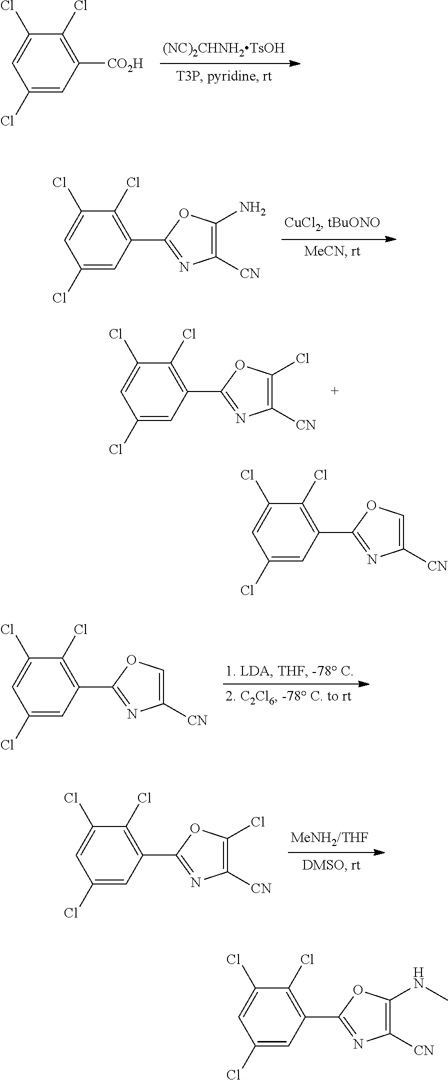

The synthetic route described below is described in Rai, G., et al. J. Med. Chem., 2014, 57, 4035-4048.

### Step 1. 5-Amino-2-(2,3,5-trichlorophenyl) oxazole-4-carbonitrile

1-Propanephosphonic anhydride solution (T3P, 54 mL, 50% solution in ethyl acetate, 93.1 mmol, 2.1 equiv) was added drop-wise to a mixture of 2,3,5-trichlorobenzoic acid (10.0 g, 44.3 mmol) and aminomalononitrile p-toluenesulfonate (11.8 g, 46.6 mmol, 1.05 equiv) in anhydrous pyridine (200 mL, 20 volumes of acid) at room temperature under nitrogen, after which the resulting mixture was stirred at room temperature for 22 h. The mixture was poured into water (1.2 L), stirred for 20 min and the solids were collected by filtration under reduced pressure, washing with water (2×30 mL), and dried overnight under high vacuum at 50° C. to produce 5-amino-2-(2,3,5-trichlorophenyl) oxazole-4-carbonitrile (11.04 g, 86%) as a light pink solid: ¹H NMR (300 MHz, CDCl₃) δ 7.78 (d, J=2.5 Hz, 1H), 7.56 (d, J=2.3 Hz, 1H), 5.22 (br s, 2H) ppm; ESI MS m/z 288 [M+H]⁺.

### Step 2. 5-Chloro-2-(2,3,5-trichlorophenyl) oxazole-4-carbonitrile from 5-amino-2-(2,3,5-trichlorophenyl) oxazole-4-carbonitrile

tert-Butyl nitrite (1.7 mL, 13.9 mmol, 2.0 equiv) was added slowly drop-wise to a suspension of copper (II) chloride (1.86 g, 13.9 mmol, 2.0 equiv) and 5-amino-2-(2,3,5-trichlorophenyl) oxazole-4-carbonitrile (2.0 g, 6.9 mmol) in anhydrous acetonitrile (40 mL, 20 volumes of oxazole) at room temperature under nitrogen, after which the resulting mixture was stirred at room temperature for 30 min. The mixture was diluted with 1N hydrogen chloride solution (100 mL) and extracted with ethyl acetate (80 mL). The organic extract was washed with saturated aqueous sodium chloride solution (60 mL), dried over anhydrous magnesium sulfate, filtered and the solvents were removed under reduced pressure. The residue was purified by column chromatography on silica gel, eluting with ethyl acetate/heptane (gradient from 0:100 to 10:90), to provide 5-chloro-2-(2,3,5-trichlorophenyl) oxazole-4-carbonitrile (548 mg, 26%) as an off-white solid: ¹H NMR (300 MHz, CDCl₃) δ 7.86 (d, J=2.4 Hz, 1H), 7.68 (d, J=2.4 Hz, 1H) ppm; ESI MS m/z 307 [M+H]⁺. Continued elution led to the isolation of byproduct 2-(2,3,5-trichlorophenyl) oxazole-4-carbonitrile (215 mg, 11%) as a light yellow solid: ¹H NMR (300 MHz, CDCl₃) δ 8.33 (s, 1H), 7.91 (d, J=2.1 Hz, 1H), 7.67 (d, J=2.4 Hz, 1H) ppm; ESI MS m/z 274 [M+H]⁺.

### Step 2a. 5-Chloro-2-(2,3,5-trichlorophenyl) oxazole-4-carbonitrile from 2-(2,3,5-trichlorophenyl) oxazole-4-carbonitrile

n-Butyllithium (1.24 mL, 2.5 M solution in hexanes, 3.11 mmol, 1.05 equiv) was added drop-wise to a solution of 2-(2,3,5-trichlorophenyl) oxazole-4-carbonitrile (810 mg, 2.96 mmol) in anhydrous THF (20 mL, 25 volumes of oxazole) at −78° C. under nitrogen, after which the mixture was stirred at −78° C. for 15 min. Solid hexachloroethane (757 mg, 3.20 mmol, 1.1 equiv) was added in one portion, after which the mixture was slowly warmed to room temperature, stirring for a total of 1 h. The mixture was diluted with saturated aqueous ammonium chloride solution (10 mL) and extracted with ethyl acetate (2×80 mL). The combined organic extracts were dried over anhydrous sodium sulfate, filtered and the solvents were removed under reduced pressure. The residue was purified by column chromatography on silica gel, eluting with ethyl acetate/heptane (gradient from 0:100 to 15:85), to provide 5-chloro-2-(2,3,5-trichlorophenyl) oxazole-4-carbonitrile (230 mg, 25%) as a light yellow solid.

### Step 3. 5-(Methylamino)-2-(2,3,5-trichlorophenyl) oxazole-4-carbonitrile

Methylamine (8.9 mL, 2.0 M solution in THF, 17.8 mmol, 10.0 equiv) was added to a solution of 5-chloro-2-(2,3,5-trichlorophenyl) oxazole-4-carbonitrile (548 mg, 1.80 mmol) in anhydrous DMSO (16 mL, 30 volumes of oxazole) at room temperature under nitrogen, after which the resulting mixture was stirred at room temperature for 4 h. The mixture was poured into saturated aqueous sodium chloride solution (100 mL) and extracted with ethyl acetate (2×70 mL). The combined organic extracts were dried over anhydrous sodium sulfate, filtered and the solvents were removed under reduced pressure. The residue was purified by column chromatography on silica gel, eluting with ethyl acetate/heptane (gradient from 0:100 to 40:60), to provide 5-(methylamino)-2-(2,3,5-trichlorophenyl) oxazole-4-carbonitrile (cmpd. 1, 340 mg, 63%) as an off-white solid: ¹H NMR (500 MHZ, DMSO-d₆) δ 8.57 (q, J=4.8 Hz, 1H), 7.99 (d, J=2.5 Hz, 1H), 7.87 (d, J=2.4 Hz, 1H), 3.00 (d, J=4.8 Hz, 3H) ppm; ESI MS m/z 302 [M+H]⁺.

### Preparation of Intermediate Amines

### Preparation of 2-(2-isopropoxyethoxy) ethan-1-amine

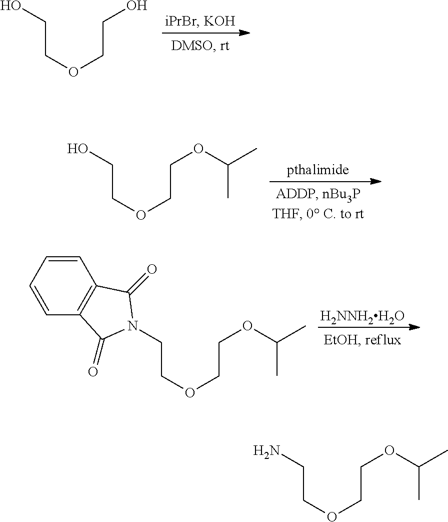

### Step 1. 2-(2-Isopropoxyethoxy) ethan-1-ol

Diethylene glycol (8.63 g, 81.30 mmol) was charged with anhydrous DMSO (110 mL) at room temperature under nitrogen and the solution stirred for 20 min. Potassium hydroxide (5.02 g, 89.43 mmol) and 2-bromopropane (3.81 mL, 40.62 mmol) were sequentially added, after which the mixture was stirred at room temperature 18 h. The mixture was diluted with water (500 mL), treated with 2N HCl (5 mL) and extracted with ethyl acetate (3×80 mL). The combined organic extracts were washed with saturated aqueous sodium chloride solution (300 mL), dried over anhydrous sodium sulfate, filtered and the solvents were removed under reduced pressure to provide 2-(2-isopropoxyethoxy) ethanol (339 mg, 6%) as a pale yellow oil: ¹H NMR (300 MHz, CDCl₃) δ 3.74-3.57 (m, 9H), 1.18 (d, J=6.1 Hz, 6H) ppm.

### Step 2. 2-(2-(2-Isopropoxyethoxy)ethyl) isoindoline-1,3-dione

1,1′-(Azodicarbonyl)dipiperidine (638 mg, 2.52 mmol) and phthalimide (370 mg, 2.74 mmol) were added sequentially to a solution of 2-(2-isopropoxyethoxy) ethanol (339 mg, 2.29 mmol) in anhydrous THF (20.8 mL) at 0° C. (ice/water bath) under nitrogen, after which tri-n-butylphosphine (0.68 mL, 2.74 mmol) was added drop-wise. The resulting solution was slowly warmed to room temperature, stirring for a total of 18 h. The mixture was diluted with water (100 mL) and extracted with ethyl acetate (3×20 mL). The combined organic extracts were washed with saturated aqueous sodium chloride solution (60 mL), dried over anhydrous sodium sulfate, filtered, and the solvents were removed under reduced pressure. The residue was purified by column chromatography on silica gel, eluting with ethyl acetate/dichloromethane (gradient from 0:100 to 50:50), to provide 2-(2-(2-isopropoxyethoxy)ethyl) isoindoline-1,3-dione (471 mg, 74%) as an off-white solid: ¹H NMR (300 MHz, CDCl₃) δ 7.86-7.83 (m, 2H), 7.72-7.70 (m, 2H), 3.92-3.89 (m, 2H), 3.77-3.73 (m, 2H), 3.64-3.59 (m, 3H), 3.55-3.51 (m, 2H), 1.09 (d, J=6.1 Hz, 6H) ppm.

### Step 3. 2-(2-Isopropoxyethoxy) ethan-1-amine

Hydrazine monohydrate (0.11 mL, 3.40 mmol) was added to a solution of 2-(2-(2-isopropoxyethoxy)ethyl) isoindoline-1,3-dione (471 mg, 1.70 mmol) in ethanol (15.5 mL) at room temperature under nitrogen, after which the mixture was heated to reflux to stir for 2 h. The mixture was cooled to room temperature and filtered under reduced pressure to remove solid byproducts. The filtrate solvents were removed under reduced pressure to provide 2-(2-isopropoxyethoxy) ethanamine (256 mg, >99%) as a colorless oil: ¹H NMR (300 MHz, CDCl₃) δ 3.61-3.56 (m, 5H), 3.51 (t, J=5.1 Hz, 2H), 2.86 (t, J=5.2 Hz, 2H), 1.17 (d, J=6.1 Hz, 6H) ppm.

### Preparation of 2-(2-(pyridin-4-yloxy) ethoxy) ethan-1-amine

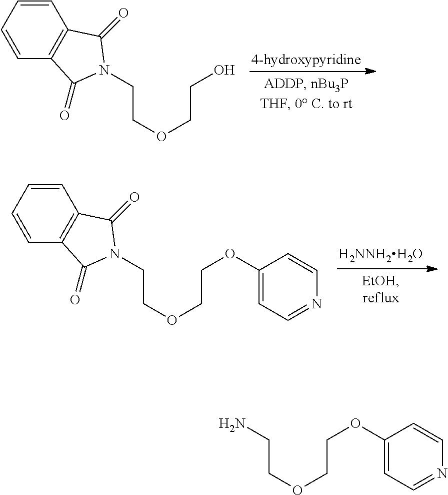

### Step 1. 2-(2-(3-(Pyridin-4-yl) propoxy)ethyl) isoindoline-1,3-dione

4-Hydroxypyridine (1.94 g, 20.41 mmol) and 1,1′-(azodicarbonyl)dipiperidine (6.44 g, 25.51 mmol) were added sequentially to a solution of 2-(2-(2-hydroxyethoxy)ethyl) isoindoline-1,3-dione (4.00 g, 17.00 mmol) in anhydrous THF (94.4 mL) at 0° C. (ice/water bath) under nitrogen, after which tri-n-butylphosphine (6.37 mL, 25.51 mmol) was added drop-wise. The mixture was slowly warmed to room temperature, stirring for a total of 18 h. The mixture was diluted with water (200 mL) and extracted with ethyl acetate (3×100 mL). The combined organic extracts were washed with saturated aqueous sodium chloride solution (60 mL), dried over anhydrous sodium sulfate, filtered, and the solvents were removed under reduced pressure. The residue was dissolved in dichloromethane and cooled to 0° C. for 20 min, after which the solids were removed by filtration under reduced pressure. The filtrate solvents were removed under reduced pressure and the residue was purified by column chromatography on silica gel, eluting with methanol/ethyl acetate (gradient from 0:100 to 40:60), to provide 2-(2-(3-(pyridin-4-yl) propoxy)ethyl) isoindoline-1,3-dione (1.48 g, 28%) as an off-white solid: ¹H NMR (300 MHz, CDCl₃) δ 8.36-8.34 (m, 2H), 7.83-7.80 (m, 2H), 7.72-7.69 (m, 2H), 6.73-6.71 (m, 2H), 4.12-4.09 (m, 2H), 3.94-3.90 (m, 2H), 3.87-3.80 (m, 4H) ppm.

### Step 2. 2-(2-(Pyridin-4-yloxy) ethoxy) ethan-1-amine

Hydrazine monohydrate (0.30 mL, 9.50 mmol) was added to a solution of 2-(2-(3-(pyridin-4-yl) propoxy)ethyl) isoindoline-1,3-dione (1.48 g, 4.75 mmol) in ethanol (43 mL) at room temperature under nitrogen, after which the mixture was heated to reflux to stir for 1 h. The mixture was cooled to room temperature and filtered under reduced pressure to remove solid byproducts. The filtrate solvents were removed under reduced pressure and the residue was purified by column chromatography on silica gel, eluting with methanol/dichloromethane (gradient from 0:100 to 4:96), to provide 2-(2-(pyridin-4-yloxy) ethoxy) ethan-1-amine (189 mg, 22%) as a colorless oil: ¹H NMR (300 MHz, CDCl₃) δ 8.44-8.42 (m, 2H), 6.84-6.82 (m, 2H), 4.20-4.16 (m, 2H), 3.86-3.83 (m, 2H), 3.58 (t, J=5.1 Hz, 2H), 2.90 (t, J=5.1 Hz, 2H) ppm.

### Preparation of 2-(2-(2-(pyridin-4-yloxy) ethoxy) ethoxy) ethan-1-amine

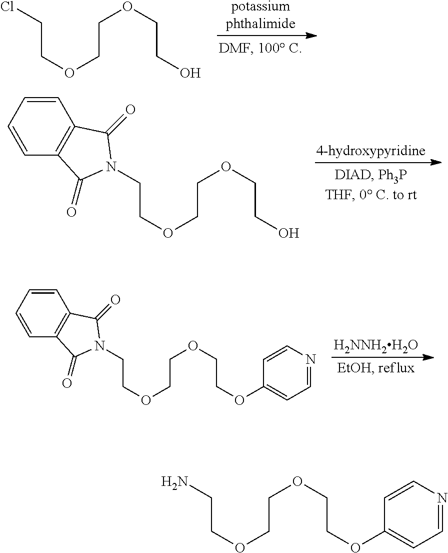

### Step 1. 2-(2-(2-(2-Hydroxyethoxy) ethoxy)ethyl) isoindoline-1,3-dione

Potassium phthalimide (2.42 g, 13.05 mmol) was added to a solution of 2-(2-(2-chloroethoxy) ethoxy) ethan-1-ol (1.72 mL, 11.86 mmol) in anhydrous DMF (14.5 mL) at room temperature under nitrogen, after which the mixture was heated to 100° C. to stir for 18 h. The mixture was cooled to room temperature and the solids were removed by filtration under reduced pressure. The filtrate solvents were removed under reduced pressure and the residue was diluted with water (60 mL) and extracted with dichloromethane (2×40 mL). The combined organic extracts were washed with saturated aqueous sodium chloride solution (60 mL), dried over anhydrous sodium sulfate, filtered, and the solvents were removed under reduced pressure to provide 2-(2-(2-(2-hydroxyethoxy) ethoxy)ethyl) isoindoline-1,3-dione (2.66 g, 80%) as a pale yellow oil: ¹H NMR (300 MHZ, CDCl₃) δ 7.88-7.80 (m, 2H), 7.79-7.68 (m, 2H), 3.93-3.90 (m, 2H), 3.78-3.74 (m, 2H), 3.69-3.59 (m, 6H), 3.55-3.50 (m, 2H) ppm.

### Step 2. 2-(2-(2-(2-(Pyridin-4-yloxy) ethoxy) ethoxy)ethyl) isoindoline-1,3-dione

Triphenylphosphine (2.33 g, 8.92 mmol) was added to a solution of 2-(2-(2-(2-hydroxyethoxy) ethoxy)ethyl) isoindoline-1,3-dione (1.66 g, 5.94 mmol), 4-hydroxypyridine (0.85 g, 8.92 mmol) and diisopropyl azodicarboxylate (1.75 mL, 8.92 mmol) in anhydrous THF (30 mL) at 0° C. (ice/water bath) under nitrogen, after which the mixture was slowly warmed to room temperature, stirring for a total of 18 h. The mixture was diluted with water (100 mL) and extracted with ethyl acetate (3×50 mL). The combined organic extracts were washed with saturated aqueous sodium chloride solution (150 mL), dried over anhydrous sodium sulfate, filtered, and the solvents were removed under reduced pressure. The residue was purified by column chromatography on silica gel, eluting with methanol/ethyl acetate (gradient from 0:100 to 40:60), to provide 2-(2-(2-(2-(pyridin-4-yloxy) ethoxy) ethoxy)ethyl) isoindoline-1,3-dione (664 mg, 31%) as an off-white solid: ¹H NMR (300 MHz, CDCl₃) δ 8.43-8.39 (m, 2H), 7.84-7.78 (m, 2H), 7.71-7.66 (m, 2H), 6.81-6.78 (m, 2H), 4.14-4.07 (m, 2H), 3.92-3.86 (m, 2H), 3.85-3.79 (m, 2H), 3.77-3.70 (m, 2H), 3.67 (s, 4H) ppm.

### Step 3. 2-(2-(2-(Pyridin-4-yloxy) ethoxy) ethoxy) ethan-1-amine

Hydrazine monohydrate (0.12 mL, 3.72 mmol) was added to a solution of 2-(2-(2-(2-(pyridin-4-yloxy) ethoxy) ethoxy)ethyl) isoindoline-1,3-dione (664 mg, 1.86 mmol) in ethanol (20 mL) at room temperature under nitrogen, after which the mixture was heated to reflux to stir for 1.5 h. The mixture was cooled to room temperature and filtered under reduced pressure to remove solid byproducts. The filtrate solvents were removed under reduced pressure and the residue was purified by column chromatography on silica gel, eluting with methanol/dichloromethane (gradient from 0:100 to 100:0), to provide 2-(2-(2-(pyridin-4-yloxy) ethoxy) ethoxy) ethan-1-amine (174 mg, 41%) as a colorless oil: ¹H NMR (300 MHz, CDCl₃) δ 8.34-8.32 (m, 2H), 7.01-6.99 (m, 2H), 4.25-4.22 (m, 2H), 3.88-3.85 (m, 2H), 3.72-3.69 (m, 2H), 3.65-3.61 (m, 2H), 3.51 (t, J=5.1 Hz, 2H), 2.78 (t, J=5.4 Hz, 2H) ppm.

### Preparation of 2-(2-(2-fluoroethoxy) ethoxy) ethan-1-amine

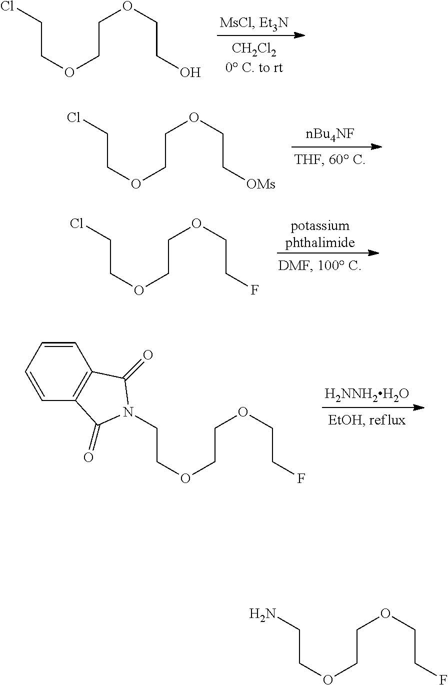

### Step 1. 2-(2-(2-Chloroethoxy) ethoxy)ethyl methanesulfonate

Methanesulfonyl chloride (1.84 mL, 23.72 mmol) was added slowly drop-wise to a solution of 2-(2-(2-chloroethoxy) ethoxy) ethan-1-ol (1.72 mL, 11.86 mmol) and triethylamine (4.93 mL, 35.58 mmol) in anhydrous dichloromethane at 0° C. (ice/water bath) under nitrogen, after which the mixture was slowly warmed to room temperature, stirring for a total of 3.5 h. The mixture was diluted with water (100 mL) and the organic layer was collected, washed with saturated aqueous sodium chloride solution (2×100 mL), dried over anhydrous sodium sulfate, filtered, and the solvents were removed under reduced pressure to provide 2-(2-(2-chloroethoxy) ethoxy)ethyl methanesulfonate (2.92 g, >99%) as a colorless oil: ¹H NMR (300 MHz, CDCl₃) δ 4.40-4.37 (m, 2H), 3.80-3.73 (m, 4H), 3.68 (s, 4H), 3.65-3.61 (m, 2H), 3.08 (s, 3H) ppm.

### Step 2. 1-Chloro-2-(2-(2-fluoroethoxy) ethoxy) ethane

Tetrabutylammonium fluoride (23.72 mL, 23.72 mmol, 1.0 M solution in THF) was added to a solution of 2 (2.92 g, 11.86 mmol) in anhydrous THF (1.0 mL) at room temperature under nitrogen, after which the mixture was heated to 60° C. to stir for 18 h. The mixture was cooled to room temperature and the solvents were removed under reduced pressure. The residue was dissolved in dichloromethane (25 mL), washed with water (2×100 mL) and saturated aqueous sodium chloride solution (100 mL), dried over anhydrous sodium sulfate, filtered, and the solvents were removed under reduced pressure to provide 1-chloro-2-(2-(2-fluoroethoxy) ethoxy) ethane (1.44 g, 72%) as an amber oil: ¹H NMR (300 MHZ, CDCl₃) δ 4.66-4.47 (m, 2H), 3.88-3.79 (m, 2H), 3.77-3.74 (m, 2H), 3.72-3.70 (m, 4H), 3.67-3.62 (m, 2H) ppm.

### Step 3. 2-(2-(2-(2-Fluoroethoxy) ethoxy)ethyl) isoindoline-1,3-dione

Potassium phthalimide (1.71 g, 9.26 mmol) was added to a solution of 1-chloro-2-(2-(2-fluoroethoxy) ethoxy) ethane (1.44 g, 8.41 mmol) in anhydrous DMF (10.4 mL) at room temperature under nitrogen, after which the mixture was heated to 100° C. to stir for 18 h. The mixture cooled to room temperature and the solids were removed by filtration under reduced pressure. The filtrate solvents were removed under reduced pressure and the residue was diluted with water (100 mL) and extracted with ethyl acetate (3×30 mL). The combined organic extracts were washed with saturated aqueous sodium chloride solution (60 mL), dried over anhydrous sodium sulfate, filtered, and the solvents were removed under reduced pressure to provide 2-(2-(2-(2-fluoroethoxy) ethoxy)ethyl) isoindoline-1,3-dione (441 mg, 19%) as an amber oil: ¹H NMR (300 MHz, CDCl₃) δ 7.87-7.82 (m, 2H), 7.75-7.67 (m, 2H), 4.58-4.39 (m, 2H), 3.93-3.86 (m, 2H), 3.77-3.72 (m, 2H), 3.70-3.62 (m, 6H) ppm.

### Step 4. 2-(2-(2-Fluoroethoxy) ethoxy) ethan-1-amine

Hydrazine monohydrate (0.10 mL, 3.14 mmol) was added to a solution of 2-(2-(2-(2-fluoroethoxy) ethoxy)ethyl) isoindoline-1,3-dione (441 mg, 1.57 mmol) in ethanol (14 mL) at room temperature under nitrogen, after which the mixture was heated to reflux to stir for 1 h. The mixture was cooled to room temperature and filtered under reduced pressure to remove solid byproducts. The filtrate solvents were removed under reduced pressure to provide 2-(2-(2-fluoroethoxy) ethoxy) ethan-1-amine (146 mg, 62%) as a colorless oil: ¹H NMR (300 MHZ, CDCl₃) δ (300 MHZ, CDCl₃) δ 4.67-4.48 (m, 2H), 3.72-3.63 (m, 6H), 3.52 (t, J=5.1 Hz, 2H), 2.88 (t, J=5.2 Hz, 2H) ppm.

### Preparation of 2-(2-(3,3-difluoroazetidin-1-yl) ethoxy) ethan-1-amine

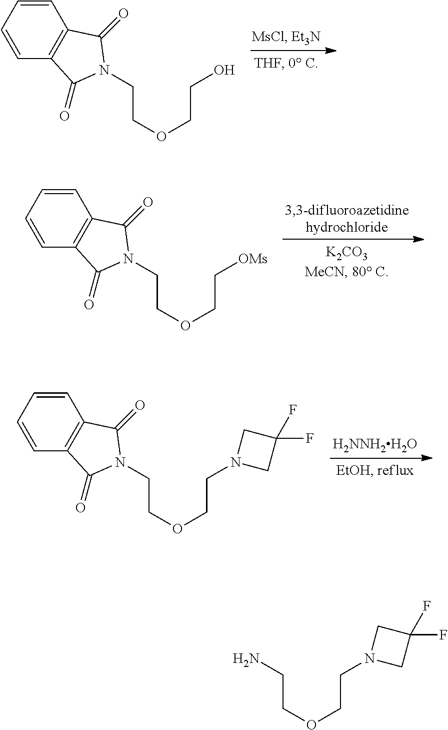

### Step 1. 2-(2-(1,3-Dioxoisoindolin-2-yl) ethoxy)ethyl methanesulfonate

Triethylamine (0.44 mL, 3.18 mmol) and methanesulfonyl chloride (0.17 mL, 2.22 mmol) were added to a solution of 2-(2-(2-hydroxyethoxy)ethyl) isoindoline-1,3-dione (0.50 g, 2.12 mmol) in anhydrous THF (20 mL) at 0° C. (ice/water bath) under nitrogen, after which the mixture was stirred at 0° C. for 2 h. The mixture was diluted with water (75 mL), warmed to room temperature and extracted with ethyl acetate (3×75 mL). The combine organic extracts were washed with saturated aqueous sodium chloride solution (2×50 mL), dried over anhydrous sodium sulfate, filtered, and the solvents were removed under reduced pressure to provide 2-(2-(1,3-dioxoisoindolin-2-yl) ethoxy)ethyl methanesulfonate (0.61 g, 91%) as an off-white solid: ¹H NMR (300 MHz, CDCl₃) δ 7.88-7.82 (m, 2H), 7.77-7.69 (m, 2H), 4.34-4.28 (m, 2H), 3.91 (t, J=5.5 Hz, 2H), 3.79-3.70 (m, 4H), 3.00 (s, 3H) ppm.

### Step 2. 2-(2-(2-(3,3-Difluoroazetidin-1-yl) ethoxy)ethyl) isoindoline-1,3-dione

3,3-Difluoroazetidine hydrochloride (0.13 g, 1.02 mmol) and potassium carbonate (0.42 g, 3.06 mmol) were added to a solution of 2-(2-(1,3-dioxoisoindolin-2-yl) ethoxy)ethyl methanesulfonate (0.30 g, 0.50 mmol) in anhydrous acetonitrile (3.5 mL) in a sealed reaction vessel at room temperature under nitrogen, after which the vessel was sealed and the mixture was heated to 80° C. to stir for 18 h. The mixture was cooled to room temperature and the solids were removed by filtration under reduced pressure. The filtrate solvents were removed under reduced pressure and the residue was purified by column chromatography on silica gel, eluting with ethyl acetate/heptane (gradient from 0:100 to 50:50), to provide 2-(2-(2-(3,3-difluoroazetidin-1-yl) ethoxy)ethyl) isoindoline-1,3-dione (194 mg, 73%) as a white solid: ESI MS m/z 311 [M+H]⁺.

### Step 3. 2-(2-(Pyridin-4-yloxy) ethoxy) ethan-1-amine

Hydrazine monohydrate (61 μL, 1.05 mmol) was added to a solution of 2-(2-(2-(3,3-difluoroazetidin-1-yl) ethoxy)ethyl) isoindoline-1,3-dione (194 mg, 0.63 mmol) in ethanol (6 mL) at room temperature under nitrogen, after which the mixture was heated to reflux to stir for 5 h. The mixture was cooled to room temperature and filtered under reduced pressure to remove solid byproducts. The filtrate solvents were removed under reduced pressure to provide 2-(2-(pyridin-4-yloxy) ethoxy) ethan-1-amine (111 mg, 98%) as an off-white semi-solid: ¹H NMR (300 MHz, CDCl₃) δ 3.68 (t, J=12.1 Hz, 4H), 3.55-3.48 (m, 4H), 3.26 (br s, 2H), 2.97-2.90 (m, 2H), 2.82-2.76 (m, 2H) ppm; ESI MS m/z 181 [M+H]⁺.

### Preparation of N-(2-(2-aminoethoxy)ethyl)-2,2,2-trifluoro-N-methylethan-1-amine

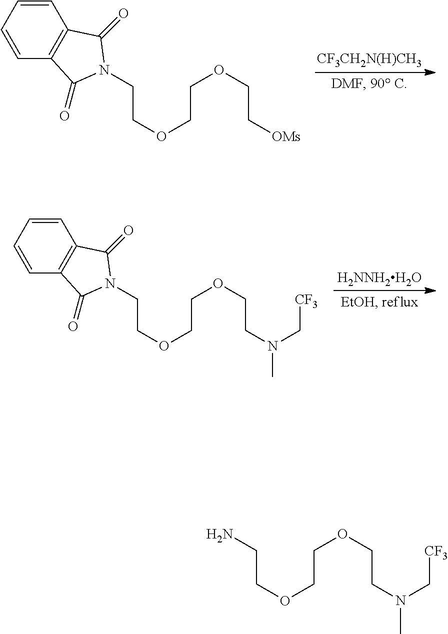

### Step 1. 2-(2-(2-(Methyl (2,2,2-trifluoroethyl)amino) ethoxy)ethyl) isoindoline-1,3-dione

N-Methyl N-trifluoroethylamine (0.50 g, 4.42 mmol) was added to a solution of 2-(2-(1,3-dioxoisoindolin-2-yl) ethoxy)ethyl methanesulfonate (0.28 g, 0.88 mmol) in anhydrous DMF (5.0 mL) in a sealed reaction vessel at room temperature under nitrogen, after which the vessel was sealed and the mixture was heated to 80° C. to stir for 96 h. The mixture was cooled to room temperature, diluted with water (25 mL) and extracted with ethyl acetate (3×40 mL). The combined organic extracts were washed sequentially with 10% aqueous lithium chloride solution (2×30 mL) and saturated aqueous sodium chloride solution (2×30 mL), dried over anhydrous sodium sulfate, filtered, and the solvents were removed under reduced pressure. The residue was purified by column chromatography on silica gel, eluting with ethyl acetate/heptane (gradient from 0:100 to 50:50), to provide 2-(2-(2-(methyl (2,2,2-trifluoroethyl)amino) ethoxy)ethyl) isoindoline-1,3-dione (174 mg, 59%) as a colorless oil: ¹H NMR (300 MHz, CDCl₃) δ 7.88-7.81 (m, 2H), 7.75-7.68 (m, 2H), 3.92 (t, J=5.6 Hz, 2H), 3.68 (t, J=5.6 Hz, Hz, 2H), 3.58 (t, J=5.4 Hz, 2H), 3.04 (q, J=9.6 Hz, 2H), 2.75 (t, J=5.4 Hz, 2H), 2.42 (s, 3H) ppm.

### Step 2. N-(2-(2-Aminoethoxy)ethyl)-2,2,2-trifluoro-N-methylethan-1-amine

Hydrazine monohydrate (50 μL, 1.05 mmol) was added to a solution of 2-(2-(2-(methyl (2,2,2-trifluoroethyl)amino) ethoxy)ethyl) isoindoline-1,3-dione (174 mg, 0.53 mmol) in ethanol (5 mL) at room temperature under nitrogen, after which the mixture was heated to reflux to stir for 4 h. The mixture was cooled to room temperature and filtered under reduced pressure to remove solid byproducts. The filtrate solvents were removed under reduced pressure to provide N-(2-(2-aminoethoxy)ethyl)-2,2,2-trifluoro-N-methylethan-1-amine (97 mg, 92%) as an off-white semi-solid: ¹H NMR (300 MHz, CDCl₃) δ 3.62-3.55 (m, 2H), 3.47 (t, J=5.2 Hz, 2H), 3.12 (q, J=9.6 Hz, 2H), 2.86 (t, J=5.3 Hz, 2H), 2.83-2.65 (m, 2H), 2.50 (s, 3H) ppm.

### Preparation of 4-(pyridin-4-ylmethoxy) but-2-yn-1-amine ditosylate

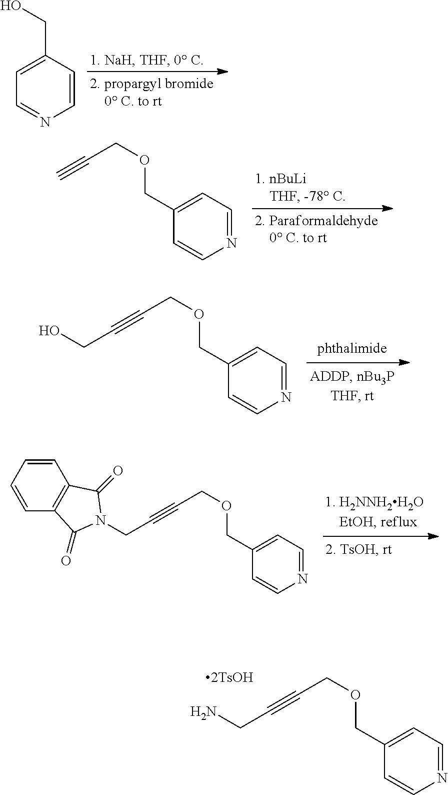 [SMILES: OCc1ccncc1 ; C#CCOCc1ccncc1 ; OCC#CCOCc1ccncc1 ; O=C1c2ccccc2C(=O)N1CC#CCOCc1ccncc1 ; NCC#CCOCc1ccncc1]

### Step 1. 4-((Prop-2-yn-1-yloxy)methyl) pyridine

Sodium hydride (1.47 g, 36.6 mmol, 60% suspension in mineral oil) was added portion-wise to a solution of 4-pyridinemthanol (2.00 g, 18.3 mmol) in anhydrous THF (200 mL) at 0° C. (ice/water bath) under nitrogen, after which the mixture was stirred at 0° C. for 30 min. Propargyl bromide (4.0 mL, 36.6 mmol) was added drop-wise, after which the mixture was slowly warmed to room temperature, stirring for a total of 23 h. The mixture was diluted saturated aqueous ammonium chloride solution (5 mL) and then with water (300 mL) and extracted with ethyl acetate (3×100 mL). The combined organic extracts were washed with saturated aqueous sodium chloride solution (200 mL), dried over anhydrous sodium sulfate, filtered, and the solvents were removed under reduced pressure. The residue was purified by column chromatography on silica gel, eluting with methanol/dichloromethane (gradient from 0:100 to 10:90), to provide 4-((prop-2-yn-1-yloxy)methyl) pyridine (1.89 g, 72%) as an orange oil: ¹H NMR (300 MHz, CDCl₃) δ 8.58-8.56 (m, 2H), 7.29-7.27 (m, 2H), 4.63 (s, 2H), 4.24 (d, J=2.4 Hz, 2H), 2.51 (t, J=2.3 Hz, 1H) ppm.

### Step 2. 4-(Pyridin-4-ylmethoxy) but-2-yn-1-ol

n-Butyl lithium (6.16 mL, 15.4 mmol, 2.5 M solution in hexanes) was added drop-wise to a solution of 4-((prop-2-yn-1-yloxy)methyl) pyridine (1.89 g, 12.8 mmol) in anhydrous THF (27.8 mL) at −78° C. under nitrogen, after which the mixture was stirred at −78° C. for 1 h and then warmed to 0° C. (ice/water bath). Paraformaldehyde (0.91 g) was added in one portion, after which the mixture was slowly warmed to room temperature, stirring for a total of 12 h. The reaction was quenched with saturated aqueous ammonium chloride solution (5 mL) and then with water (200 mL) and extracted with ethyl acetate (3×70 mL). The combined organic extracts were washed with saturated aqueous sodium chloride solution (200 mL), dried over anhydrous sodium sulfate, filtered, and the solvents were removed under reduced pressure. The residue was purified by column chromatography on silica gel, eluting with methanol/dichloromethane (gradient from 0:100 to 10:90), to provide 4-(pyridin-4-ylmethoxy) but-2-yn-1-ol (0.99 g, 44%) as an opaque oil: ¹H NMR (300 MHZ, CDCl₃) 8.48-8.46 (m, 2H), 7.27-7.24 (m, 2H), 4.54 (s, 2H), 4.18-4.16 (m, 4H) ppm.

### Step 3. 2-(4-(Pyridin-4-ylmethoxy) but-2-yn-1-yl) isoindoline-1,3-dione

Tri-n-butylphosphine (1.67 mL, 6.70 mmol) was added drop-wise to a solution of 1,1′-(azodicarbonyl)dipiperidine (1.55 g, 6.14 mmol), phthalimide (0.90 g, 6.14 mmol) and 4-(pyridin-4-ylmethoxy) but-2-yn-1-ol (0.99 g, 5.58 mmol) in anhydrous THF (27.9 mL) at 0° C. (ice/water bath) under nitrogen, after which the mixture was slowly warmed to room temperature, stirring for a total of 18 h. The mixture was diluted with water (200 mL) and extracted with ethyl acetate (3×70 mL). The combined organic extracts were washed with saturated aqueous sodium chloride solution (100 mL), dried over anhydrous sodium sulfate, filtered, and the solvents were removed under reduced pressure. The residue was purified by column chromatography on silica gel, eluting with ethyl acetate/dichloromethane (gradient from 0:100 to 60:40), to provide 2-(4-(pyridin-4-ylmethoxy) but-2-yn-1-yl) isoindoline-1,3-dione (920 mg, 54%) as an off-white solid: ¹H NMR (300 MHz, CDCl₃) δ 8.54 (d, J=5.7 Hz, 2H), 7.89-7.87 (m, 2H), 7.76-7.73 (m, 2H), 7.25 (d, J=5.9 Hz, 2H), 4.58 (s, 2H), 4.51 (t, J=1.8 Hz, 2H), 4.22 (t, J=1.8 Hz, 2H) ppm.

### Step 4. 4-(Pyridin-4-ylmethoxy) but-2-yn-1-amine ditosylate

Hydrazine monohydrate (0.09 mL, 2.93 mmol) was added to a solution of 2-(4-(pyridin-4-ylmethoxy) but-2-yn-1-yl) isoindoline-1,3-dione (448 mg, 1.46 mmol) in ethanol (13 mL) at room temperature under nitrogen, after which the mixture was heated to reflux to stir for 1 h. The mixture was cooled to room temperature and filtered under reduced pressure to remove solid byproducts. The filtrate was treated with p-toluenesulfonic acid (505 mg, 2.93 mmol) and the solvents were removed under reduced pressure to provide 4-(pyridin-4-ylmethoxy) but-2-yn-1-amine ditosylate (809 mg, >99%) as an off-white solid: ¹H NMR (300 MHz, CDCl₃) δ 8.56-8.54 (m, 2H), 7.49-7.46 (m, 4H), 7.33-7.31 (m, 2H), 7.13-7.10 (m, 4H), 4.60 (s, 2H), 4.33 (t, J=1.8 Hz, 2H), 3.84 (t, J=1.8 Hz, 2H), 2.51-2.46 (m, 2H), 2.28 (s, 6H) ppm.

### Preparation of 4-((4-(2-morpholinoethyl)benzyl)oxy) but-2-yn-1-amine

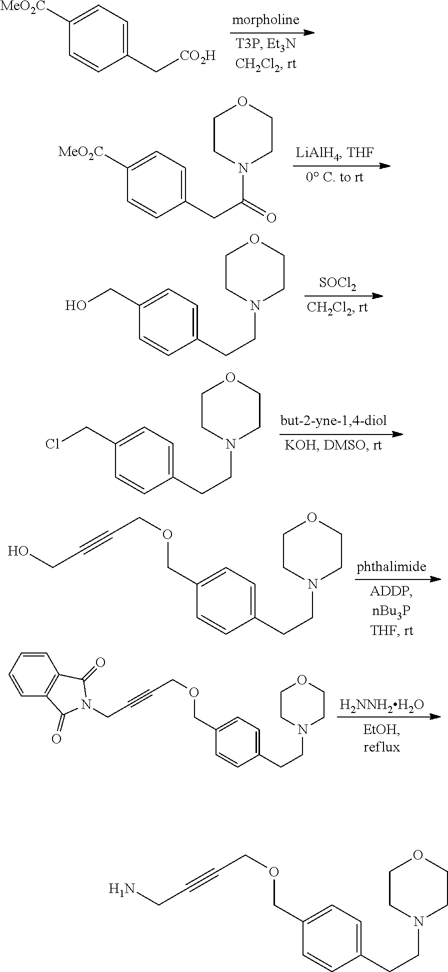

### Step 1. Methyl 4-(2-morpholino-2-oxoethyl)benzoate

Propanephosphonic acid anhydride (26.1 mL, 43.8 mmol, 50% wt. solution in ethyl acetate) was added drop-wise via addition funnel to a solution of morpholine (3.38 mL, 38.6 mmol), triethylamine (10.7 mL, 77.1 mmol) and 2-(4-(methoxycarbonyl)phenyl) acetic acid (5.00 g, 25.7 mmol) in anhydrous dichloromethane (122.3 mL) at 0° C. (ice/water bath) under nitrogen, after which the mixture was slowly warmed to room temperature, stirring for a total of 18 h. The reaction was diluted with water (200 mL) and extracted with dichloromethane (3×100 mL). The combined organic extracts were washed with saturated aqueous sodium bicarbonate solution (200 mL), dried over anhydrous sodium sulfate, filtered and the solvents were removed under reduced pressure to provide methyl 4-(2-morpholino-2-oxoethyl)benzoate (6.75 g, >99%) as an off-white solid: ¹H NMR (300 MHz, CDCl₃) δ 8.02-7.98 (m, 2H), 7.33-7.31 (m, 2H), 3.91 (s, 3H), 3.78 (s, 2H), 3.65 (s, 4H), 3.52-3.49 (m, 2H), 3.44-3.41 (m, 2H) ppm.

### Step 2. (4-(2-Morpholinoethyl)phenyl) methanol

A solution of methyl 4-(2-morpholino-2-oxoethyl)benzoate (3.00 g, 11.40 mmol) in anhydrous THF (45.6 mL) was added drop-wise via addition funnel to a solution of lithium aluminum hydride (17 mL, 34.10 mmol, 2.0 M solution in THF) in anhydrous THF (25 mL) at 0° C. (ice/water bath) under nitrogen, after which the mixture was slowly warmed to room temperature, stirring for a total of 18 h. The reaction was quenched with 2N sodium hydroxide solution (0.5 mL) and the resulting solids were removed by filtration under reduced pressure. The filtrate was diluted with water (200 mL) and extracted with ethyl acetate (3×100 mL). The combined organic extracts were washed with saturated aqueous sodium chloride solution (100 mL), dried over anhydrous sodium sulfate, filtered, and the solvents were removed under reduced pressure to provide (4-(2-morpholinoethyl)phenyl) methanol (2.81 g, >99%) as an off-white solid: ¹H NMR (300 MHz, CDCl₃) δ 7.28 (d, J=7.9 Hz, 2H), 7.16 (d, J=8.0 Hz, 2H), 4.63 (s, 2H), 3.71 (t, J=4.6 Hz, 4H), 2.80-2.74 (m, 2H), 2.58-2.47 (m, 6H) ppm.

### Step 3. 4-(4-(Chloromethyl) phenethyl) morpholine

Thionyl chloride (0.72 mL, 9.80 mmol) was added to a solution of (4-(2-morpholinoethyl)phenyl) methanol (1.81 g, 8.17 mmol) in anhydrous dichloromethane (22.7 mL) at room temperature under nitrogen and the mixture was stirred for 40 min. The solvents were removed under reduced pressure to provide 4-(4-(chloromethyl) phenethyl) morpholine (2.25 g, >99%) as a pale amber oil: ¹H NMR (300 MHz, CDCl₃) δ 7.39-7.25 (m, 4H), 4.56 (s, 2H), 4.35-4.27 (m, 2H), 4.02-3.97 (m, 2H), 3.51 (d, J=11.8 Hz, 2H), 3.33-3.15 (m, 4H), 2.98-2.85 (m, 2H) ppm.

### Step 4. 4-((4-(2-Morpholinoethyl)benzyl)oxy) but-2-yn-1-ol

Potassium hydroxide (1.16 g, 20.66 mmol) was added to a solution of 1,4-butyndiol (3.20 g, 18.78 mmol) in anhydrous DMSO (10 mL) at room temperature under nitrogen and the mixture was stirred for 15 min. A solution of 4-(4-(chloromethyl) phenethyl) morpholine (2.25 g, 9.39 mmol) in anhydrous DMSO (15.3 mL) was added and the resulting mixture was stirred at room temperature for 5.5 h. The mixture was diluted with water (200 mL) and extracted with ethyl acetate (3×75 mL). The combined organic extracts were washed with saturated aqueous sodium chloride solution (150 mL), dried over anhydrous sodium sulfate, filtered, and the solvents were removed under reduced pressure. The residue was purified by column chromatography on silica gel, eluting with methanol/dichloromethane (gradient from 0:100 to 10:90), to provide 4-((4-(2-morpholinoethyl)benzyl)oxy) but-2-yn-1-ol (1.74 g, 63%) as a colorless oil: ¹H NMR (300 MHz, CDCl₃) δ 7.28-7.26 (m, 2H), 7.20-7.17 (m, 2H), 4.55 (m, 2H), 4.32-4.31 (m, 2H), 4.20-4.19 (m, 2H), 3.74 (t, J=4.6 Hz, 4H), 2.83-2.78 (m, 2H), 2.61-2.51 (m, 6H) ppm.

### Step 5. 2-(4-((4-(2-Morpholinoethyl)benzyl)oxy) but-2-yn-1-yl) isoindoline-1,3-dione

Tri-n-butylphosphine (1.62 mL, 6.48 mmol) was added drop-wise to a solution of 1,1′-(azodicarbonyl)dipiperidine (1.50 g, 5.94 mmol), phthalimide (0.87 g, 5.94 mmol) and 4-((4-(2-morpholinoethyl)benzyl)oxy) but-2-yn-1-ol (1.56 g, 5.40 mmol) in anhydrous THF (49.1 mL) at 0° C. (ice/water bath) under nitrogen, after which the mixture was slowly warmed to room temperature, stirring for a total of 18 h. The mixture was diluted with water (150 mL) and extracted with ethyl acetate (3×50 mL). The combined organic extracts were washed with saturated aqueous sodium chloride solution (100 mL), dried over anhydrous sodium sulfate, filtered, and the solvents were removed under reduced pressure. The residue was purified by column chromatography on silica gel, eluting with methanol/dichloromethane (gradient from 0:100 to 10:90), to provide 2-(4-((4-(2-morpholinoethyl)benzyl)oxy) but-2-yn-1-yl) isoindoline-1,3-dione (2.79 g, >99%) as a white solid: ¹H NMR (300 MHz, CDCl₃) δ 7.90-7.87 (m, 2H), 7.76-7.73 (m, 2H), 7.28-7.24 (m, 2H), 7.17-7.15 (m, 2H), 4.52-4.51 (m, 4H), 4.14-4.12 (m, 2H), 3.75-3.72 (m, 4H), 2.81-2.75 (m, 2H), 2.59-2.50 (m, 6H) ppm.

### Step 6. 4-((4-(2-Morpholinoethyl)benzyl)oxy) but-2-yn-1-amine

Hydrazine monohydrate (0.43 mL, 13.34 mmol) was added to a solution of 2-(4-((4-(2-morpholinoethyl)benzyl)oxy) but-2-yn-1-yl) isoindoline-1,3-dione (2.79 g, 6.68 mmol) in ethanol (13 mL) at room temperature under nitrogen, after which the mixture was heated to reflux to stir for 1 h. The mixture was cooled to room temperature and filtered under reduced pressure to remove solid byproducts. The filtrate solvents were removed under reduced pressure and the residue was purified by column chromatography on silica gel, eluting with methanol/dichloromethane (gradient from 10:90 to 30:70), to provide 4-((4-(2-morpholinoethyl)benzyl)oxy) but-2-yn-1-amine (1.01 g, 53%) as a colorless oil: ¹H NMR (300 MHz, CDCl₃) δ 7.29-7.26 (m, 2H), 7.20-7.17 (m, 2H), 4.55 (s, 2H), 4.16 (t, J=1.8 Hz, 2H), 3.74 (t, J=4.5 Hz, 4H), 3.48 (t, J=1.8 Hz, 2H), 2.82-2.77 (m, 2H), 2.60-2.50 (m, 6H) ppm.

The following compounds were prepared in accordance with the methods and procedures similar to those described above for compound 1 (synthetic route 1 or synthetic route 2) using the intermediate amines as the starting materials

| Cmpd. | | | Mass |
|---|---|---|---|
| No. | Name | NMR | spec |
| 39 | 5-((2-Methoxyethyl)amino)-2-(2,3,5- | ¹H NMR (300 MHz, CDCl₃) δ | ESI MS |
| | trichlorophenyl)oxazole-4-carbonitrile | 7.78 (d, J = 2.4 Hz, 1H), 7.54 | m/z 346 |
| | | (d, J = 2.4 Hz, 1H), 5.38 (t, J = | [M + H]⁺ |
| | | 4.4 Hz, 1H), 4.70-3.60 (m, 4H), | |
| | | 3.42 (s, 3H) ppm | |
| 50 | 5-((2-(2-(2- | ¹H NMR (500 MHz, DMSO-d₆) | ESI MS |
| | Methoxyethoxy)ethoxy)ethyl)amino)- | δ 8.76 (t, J = 5.8 Hz, 1H), 7.99 | m/z 432 |
| | 2-(2,3,5-trichlorophenyl)oxazole- | (d, J = 2.5 Hz, 1H), 7.87 (d, J = | [M − H]⁻ |
| | 4-carbonitrile | 2.5 Hz, 1H), 3.62-3.60 (m, 2H), | |
| | | 3.57-3.50 (m, 6H), 3.48-3.46 | |
| | | (m, 2H), 3.39-3.37 (m, 2H), | |
| | | 3.20 (s, 3H) ppm | |
| 69 | 5-((2-(2-Ethoxyethoxy)ethyl)amino)- | ¹H NMR (300 MHz, CDCl₃) δ | ESI MS |
| | 2-(2,3,5-trichlorophenyl)oxazole- | 7.77 (d, J = 2.5 Hz, 1H), 7.53 | m/z 404 |
| | 4-carbonitrile | (d, J = 2.5 Hz, 1H), 5.94 (t, J = | [M + H]⁺ |
| | | 5.2 Hz, 1H), 3.78-3.74 (m, 2H), | |
| | | 3.72-3.66 (m, 4H), 3.64-3.60 | |
| | | (m, 2H), 3.59 (m, 2H), 1.25 (t, | |
| | | J = 7.0 Hz, 3H) ppm | |
| 70 | 5-((2-(2-(2- | ¹H NMR (300 MHz, CDCl₃) δ | ESI MS |
| | Fluoroethoxy)ethoxy)ethyl)amino)- | 7.76 (d, J = 2.4 Hz, 1H), 7.53 | m/z 422 |
| | 2-(2,3,5-trichlorophenyl)oxazole- | (d, J = 2.4 Hz, 1H), 5.80 (t, J = | [M + H]⁺ |
| | 4-carbonitrile | 6.4 Hz, 1H), 4.70-4.52 (m, 2H), | |
| | | 3.85-3.66 (m, 10H) ppm | |
| 49 | 5-((2-(2-Isopropoxyethoxy)ethyl)amino)- | ¹H NMR (300 MHz, CDCl₃) δ | ESI MS |
| | 2-(2,3,5-trichlorophenyl)oxazole- | 7.77 (d, J = 2.4 Hz, 1H), 7.53 | m/z 418 |
| | 4-carbonitrile | (d, J = 2.4 Hz, 1H), 6.02 (t, J = | [M + H]⁺ |
| | | 5.4 Hz, 1H), 3.78-3.74 (m, 2H), | |
| | | 3.70-3.65 (m, 5H), 3.63-3.60 | |
| | | (m, 2H), 1.22 (s, 3H), 1.20 (s, | |
| | | 3H) ppm | |
| 22 | 5-((5-Hydroxypentyl)amino)-2-(2,3,5- | ¹H NMR (500 MHz, DMSO-d₆) | ESI MS |
| | trichlorophenyl)oxazole-4-carbonitrile | δ 8.69 (t, J = 5.5 Hz, 1H), 7.98 | m/z 372 |
| | | (d, J = 2.5 Hz, 1H), 7.85 (d, J = | [M − H]⁻ |
| | | 2.5 Hz, 1H), 4.35 (t, J = 5.5 Hz, | |
| | | 1H), 3.40 (q, J = 5.5 Hz, 2H), | |
| | | 3.35 (q, J = 6.0 Hz, 2H), 1.60 | |
| | | (quint, J = 7.5 Hz, 2H), 1.45 | |
| | | (quint, J = 7.0 Hz, 2H), 1.40-1.35 | |
| | | (m, 2H) ppm | |
| 48 | 5-((4-(2-Methoxyethoxy)butyl)amino)- | ¹H NMR (500 MHz, DMSO-d₆) | ESI MS |
| | 2-(2,3,5-trichlorophenyl)oxazole- | δ 8.07 (t, J = 6.0 Hz, 1H), 7.98 | m/z 416 |
| | 4-carbonitrile | (d, J = 2.5 Hz, 1H), 7.85 (d, J = | [M − H]⁻ |
| | | 2.5 Hz, 1H), 3.49-3.46 (m, 2H), | |
| | | 3.44-3.41 (m, 4H), 3.37 (q, J = | |
| | | 6.0 Hz, 2H), 3.23 (s, 3H), | |
| | | 1.657-1.56 (m, 4H) ppm | |
| 16 | 5-((3-Hydroxypropyl)amino)-2-(2,3,5- | ¹H NMR (500 MHz, DMSO-d₆) | ESI MS |
| | trichlorophenyl)oxazole-4-carbonitrile | δ 8.65 (s, 1H), 7.98 (d, J = 2.5 | m/z 346 |
| | | Hz, 1H), 7.85 (d, J = 2.0 Hz, | [M + H]⁺ |
| | | 1H), 3.50 (t, J = 2.0 Hz, 2H), | |
| | | 3.42 (t, J = 7.0 Hz, 2H), 1.75 | |
| | | (quint, J = 5.5 Hz, 2H) ppm | |
| 17 | 5-((4-Hydroxybutyl)amino)-2-(2,3,5- | ¹H NMR (500 MHz, DMSO-d₆) | ESI MS |
| | trichlorophenyl)oxazole-4-carbonitrile | δ 8.708 (t, J = 5.0 Hz, 1H), 7.98 | m/z 360 |
| | | (d, J = 2.5 Hz, 1H), 7.85 (d, J = | [M + H]⁺ |
| | | 2.5 Hz, 1H), 4.42 (t, J = 5.0 Hz, | |
| | | 1H), 3.41 (q, J = 6.0 Hz, 2H), | |
| | | 3.35 (q, J = 6.5 Hz, 2H), 1.63 | |
| | | (quint, J = 7.0 Hz, 2H), 1.50 | |
| | | (quint, J = 6.5 Hz, 2H) ppm | |
| 28 | 5-((2-(2-Hydroxyethoxy)ethyl)amino)- | ¹H NMR (500 MHz, DMSO-d₆) | ESI MS |
| | 2-(2,3,5-trichlorophenyl)oxazole- | δ 8.74 (s, 1H), 7.99 (d, J = 2.5 | m/z 376 |
| | 4-carbonitrile | Hz, 1H), 7.87 (d, J = 2.5 Hz, | [M + H]⁺ |
| | | 1H), 4.58 (s, 1H), 3.62 (t, J = | |
| | | 5.5 Hz, 2H), 3.54-3.46 (m, 6H) | |
| | | ppm | |
| 71 | 2-(2,3,5-Trichlorophenyl)-5-((2-(2-(2,2,2- | ¹H NMR (500 MHz, DMSO-d₆) | ESI MS |
| | trifluoroethoxy)ethoxy)ethyl)amino)oxazole- | δ 8.76 (t, J = 5.9 Hz, 1H), 7.99 | m/z 458 |
| | 4-carbonitrile | (d, J = 2.5 Hz, 1H), 7.87 (d, J = | [M + H]⁺ |
| | | 2.5 Hz, 1H), 4.03 (q, J = 9.5 | |
| | | Hz, 2H), 3.72-3.69 (m, 2H), | |
| | | 3.63-3.58 (m, 4H), 3.55-3.50 | |
| | | (m, 2H) ppm | |
| 14 | 5-(allylamino)-2-(2,3,5- | ¹H NMR (300 MHz, DMSO-d₆) | ESI MS |
| | trichlorophenyl)oxazole-4-carbonitrile | δ 8.94 (s, 1H), 8.01 (d, J = 2.5 | m/z 328 |
| | | Hz, 1H), 7.87 (d, J = 2.5 Hz, | [M + H]⁺ |
| | | 1H), 5.98-5.87 (m, 1H), 5.32-5.18 | |
| | | (m, 2H), 4.00-3.98 (m, 2H) ppm | |
| 15 | (E)-5-(But-2-en-1-ylamino)-2-(2,3,5- | ¹H NMR (300 MHz, DMSO-d₆) | ESI MS |
| | trichlorophenyl)oxazole-4-carbonitrile | δ 8.86 (t, J = 6.1 Hz, 1H), 8.00 | m/z 342 |
| | | (d, J = 2.5 Hz, 1H), 7.85 (d, J = | [M + H]⁺ |
| | | 2.5 Hz, 1H), 5.79-5.67 (m, 1H), | |
| | | 5.60-5.50 (m, 1H), 3.91 (t, J = | |
| | | 5.7 Hz, 2H), 1.68 (dd, J = 6.3 | |
| | | Hz, 1.3 Hz, 3H) ppm | |
| 47 | (E)-5-((4-(Benzyloxy)but-2-en-1-yl)amino)- | ¹H NMR (500 MHz, CDCl₃-d) | ESI MS |
| | 2-(2,3,5-trichlorophenyl)oxazole-4- | δ 7.75 (d, J = 2.5 Hz, 1H), 7.52 | m/z 448 |
| | carbonitrile | (d, J = 2.5 Hz, 1H), 7.36-7.26 | [M + H]⁺ |
| | | (m, 5H), 5.95-5.90 (m, 1H), | |
| | | 5.88-5.83 (m, 1H), 5.50 (t, J = 6 | |
| | | Hz, 1H), 4.53 (s, 2H), 4.10-4.06 | |
| | | (m, 4H) ppm | |
| 46 | (E)-5-((5-Methoxypent-2-en-1-yl)amino)- | ¹H NMR (300 MHz, CDCl₃) δ | ESI MS |
| | 2-(2,3,5-trichlorophenyl)oxazole-4- | 7.77 (d, J = 2.4 Hz, 1H), 7.54 | m/z 386 |
| | carbonitrile | (d, J = 2.4 Hz, 1H), 5.85-5.78 | [M + H]⁺ |
| | | (m, 1H), 5.70-5.63 (m, 1H), | |
| | | 5.13 (t, J = 6.0 Hz, 1H), 4.09-4.05 | |
| | | (m, 2H), 3.45 (t, J = 6.3 Hz, 2H), | |
| | | 3.34 (s, 3H), 2.40-2.33 (m, 2H) ppm | |
| 45 | (E)-5-((4-Methoxybut-2-en-1-yl)amino)- | ¹H NMR (300 MHz, CDCl₃) δ | ESI MS |
| | 2-(2,3,5-trichlorophenyl)oxazole-4- | 7.77 (d, J = 2.4 Hz, 1H), 7.54 | m/z 370 |
| | carbonitrile | (d, J = 2.4 Hz, 1H), 5.94-5.81 | [M − H]⁻ |
| | | (m, 2H), 5.18 (t, J = 5.9 Hz, | |
| | | 1H), 4.12 (t, J = 4.7 Hz, 2H), | |
| | | 3.96-3.95 (m, 2H), 3.36 (s, 3H) ppm | |
| 11 | 2-(2,3-Dichloro-5-fluorophenyl)-5- | ¹H NMR (500 MHz, DMSO-d₆) | ESI MS |
| | (methylamino)oxazole-4-carbonitrile | δ 7.92 (dd J = 16.0, 2.0 Hz 2H), | m/z 286 |
| | | 2.60 (s, 3H) ppm | [M + H]⁺ |
| 2 | 2-(3,5-Dichloro-2-methylphenyl)-5- | ¹H NMR (500 MHz, DMSO-d₆) | ESI MS |
| | (methylamino)oxazole-4-carbonitrile | δ 8.42 (q, J = 5.0 Hz, 1H), 7.74 | m/z 280 |
| | | (d, J = 2.1 Hz, 1H), 7.73 (d, J = | [M − H]⁻ |
| | | 2.1 Hz, 1H), 3.00 (d, J = 5.0 | |
| | | Hz, 3H), 2.62 (s, 3H) ppm | |
| 3 | 2-(3,5-Dichloro-2-fluorophenyl)-5- | ¹H NMR (500 MHz, DMSO-d₆) | ESI MS |
| | (methylamino)oxazole-4-carbonitrile | δ 8.58 (s, 1H), 7.91 (dd, J = | m/z 284 |
| | | 5.8, 2.6 Hz, 1H), 7.80 (dd, J = | [M − H]⁻ |
| | | 5.8, 2.6 Hz, 1H), 3.00 (s, 3H) ppm | |
| 4 | 2-(3,5-Dichloro-2-(fluoromethoxy)phenyl)- | ¹H NMR (500 MHz, DMSO-d₆) | ESI MS |
| | 5-(methylamino)oxazole-4-carbonitrile | δ 8.35 (q, J = 5.1 Hz, 1H), 7.94 | m/z 332 |
| | | (d, J = 2.5 Hz, 1H), 7.86 (d, J = | [M − H]⁻ |
| | | 2.5 Hz, 1H), 7.12 (t, J = 73.5 | |
| | | Hz, 2H), 2.99 (s, 3H) ppm | |
| 10 | 2-(2,3-Dichloro-5-methylphenyl)-5- | ¹H NMR (500 MHz, DMSO-d₆) | ESI MS |
| | (methylamino)oxazole-4-carbonitrile | δ 8.46 (q, J = 4.7 Hz, 1H), 7.65 | m/z 282 |
| | | (d, J = 1.9 Hz, 1H), 7.63 (d, J = | [M + H]⁺ |
| | | 1.9 Hz, 1H), 2.99 (d, J = 4.7 | |
| | | Hz, 3H), 2.35 (s, 3H) ppm | |
| 12 | 2-(2,3-Dichloro-5-(trifluoromethyl)phenyl)- | ¹H NMR (500 MHz, DMSO-d₆) | ESI MS |
| | 5-(methylamino)oxazole-4-carbonitrile | δ 8.61 (q, J = 4.7 Hz, 1H), 8.23 | m/z 334 |
| | | (d, J = 1.8 Hz, 1H), 8.09 (d, J = | [M − H]⁻ |
| | | 1.8 Hz, 1), 3.00 (d, J = 4.7 Hz, | |
| | | 3H) ppm | |
| 13 | 2-(2,3-Dichloro-5-cyanophenyl)-5- | ¹H NMR (500 MHz, DMSO-d₆) | ESI MS |
| | (methylamino)oxazole-4-carbonitrile | δ 8.62 (s, 1H), 8.37 (d, J = 2 | m/z 291 |
| | | Hz, 1H), 8.29 ( d, J = 1.5 Hz, | [M − H]⁻ |
| | | 1H), 3.02 (s, 3H) ppm | |
| 6 | 2-(2,5-Dichloro-3-methylphenyl)-5- | ¹H NMR (500 MHz, DMSO-d₆) | ESI MS |
| | (methylamino)oxazole-4-carbonitrile | δ 8.46 (q, J = 5.0 Hz, 1H), 7.70 | m/z 280 |
| | | (d, J = 2.5 Hz, 1H), 7.63 (d, J = | [M − H]⁻ |
| | | 2.5 Hz, 1H), 2.99 (d, J = 5.0 | |
| | | Hz, 3H), 2.39 (s, 3H) ppm | |
| 9 | 2-(5-Chloro-3-methoxy-2-methylphenyl)-5- | ¹H NMR (500 MHz, DMSO-d₆) | ESI MS |
| | (methylamino)oxazole-4-carbonitrile | δ 8.48 (q, J = 5.0 Hz, 1H), 7.44 | m/z 298 |
| | | (d, J = 2.5 Hz, 1H), 7.37 (d, J = | [M + H]⁺ |
| | | 2 Hz, 1H), 3.93 (s, 3H), 2.99 (d, | |
| | | J = 5 Hz, 3H) ppm | |
| 8 | 2-(2,5-Dichloro-3-(trifluoromethyl)phenyl)- | ¹H NMR (500 MHz, DMSO-d₆) | ESI MS |
| | 5-(methylamino)oxazole-4-carbonitrile | δ 8.61 (q, J = 4.9 Hz, 1H), 8.17 | m/z 334 |
| | | (d, J = 2.5 Hz, 1H), 8.10 (d, J = | [M]⁻ |
| | | 2.5 Hz, 1H), 3.0 (d, J = 4.9 Hz, | |
| | | 3H) ppm | |
| 5 | 2-(3,5-Dichloro-2-methoxyphenyl)-5- | ¹H NMR (300 MHz, DMSO-d₆) | ESI MS |
| | (methylamino)oxazole-4-carbonitrile | δ 8.53 (m, 1H), 7.81(d, J = 2.6 | m/z 298 |
| | | Hz, 1H), 7.75 (d, J = 2.6 Hz, | [M + H]⁺ |
| | | 1H), 3.84 (s, 3H), 3.00 (d, J = | |
| | | 4.8 Hz, 3H) ppm | |
| 19 | 2-(3,5-Dichloro-2-methylpheny1)-5-((4- | ¹H NMR (500 MHz, DMSO-d₆) | ESI MS |
| | hydroxybutyl)amino)oxazole-4-carbonitrile | δ 8.50 (s, 1H), 7.73 (d, J = 2.0 | m/z 338 |
| | | Hz, 1H), 7.71 (d, J = 2.5 Hz, | [M − H]⁻ |
| | | 1H), 4.44 (s, 1H), 3.43 (t, J = | |
| | | 6.5 Hz, 2H), 3.36 (t, J = 7.0 Hz, | |
| | | 2H), 2.61 (s, 3H), 1.63 (quint, J = | |
| | | 7.0 Hz, 2H), 1.50 (q, J = 6.5 | |
| | | Hz, 2H) ppm | |
| 24 | 2-(3,5-Dichloro-2-methylphenyl)-5-((5- | ¹H NMR (500 MHz, DMSO-d₆) | ESI MS |
| | hydroxypentyl)amino)oxazole-4-carbonitrile | δ 8.58 (t, J = 5.5 Hz, 1H), 7.73 | m/z 352 |
| | | (d, J = 2.0 Hz, 1H), 7.71 (d, J = | [M − H]⁻ |
| | | 2.0 Hz, 1H), 4.35 (t, J = 5.0 Hz, | |
| | | 1H), 3.40 (q, J = 5.5 Hz, 2H), | |
| | | 3.34 (q, J = 7.0 Hz, 2H), 3.21 | |
| | | (s, 3H), 1.60 (quint, J = 7.0 Hz, | |
| | | 2H), 1.46 (quint, J = 7.0 Hz, 2H), | |
| | | 1.38 (quint, J = 7.5 Hz, 2H) ppm | |
| 34 | 2-(3,5-Dichloro-2-methylphenyl)-5-((5- | ¹H NMR (500 MHz, DMSO-d₆) | ESI MS |
| | hydroxypentyl)amino)oxazole-4-carbonitrile | δ 8.64 (t, J = 6.0 Hz, 1H), 7.37 | m/z 368 |
| | | (d, J = 2.5 Hz, 2H), 3.60 (t, J = | [M − H]⁻ |
| | | 5.0 Hz, 2H), 3.57-3.55 (m, 2H), | |
| | | 3.52 (q, J = 5.5 Hz, 2H), 3.44-3.342 | |
| | | (m, 2H), 3.21 (s, 3H), 2.62 (s, 3H) | |
| | | ppm | |
| 30 | 2-(3,5-Dichloro-2-methylphenyl)-5-((5- | ¹H NMR (500 MHz, DMSO-d₆) | ESI MS |
| | hydroxypentyl)amino)oxazole-4-carbonitrile | δ 8.62 (t, J = 5.5 Hz, 1H), 7.37 | m/z 354 |
| | | (d, J = 2.5 Hz, 2H), 4.57 (t, J = | [M − H]⁻ |
| | | 5.5 Hz, 1H), 3.62 (t, J = 5.5 Hz, | |
| | | 2H), 3.54-3.46 (m, 6H), 2.62 | |
| | | (s, 3H) ppm | |
| 41 | 2-(3,5-Dichloro-2-methylphenyl)-5-((2- | ¹H NMR (500 MHz, DMSO-d₆) | ESI MS |
| | methoxyethyl)amino)oxazole-4-carbonitrile | δ 8.66 (s, 1H), 7.71 (d, J = 2.0 | m/z 324 |
| | | Hz, 2H), 3.53 (s, 4H), 3.29 (s, | [M − H]⁻ |
| | | 3H), 2.61 (s, 3H) ppm | |
| 35 | 2-(3,5-Dichloro-2-fluorophenyl)-5-((2- | ¹H NMR (500 MHz, DMSO-d₆) | ESI MS |
| | (2-methoxyethoxy)ethyl)amino)oxazole- | 8.77 (t, J = 6.0 Hz, 1H), 7.92 | m/z 372 |
| | 4-carbonitrile | (dd, J = 6.0, 3.0 Hz, 1H), 7.81 | [M − H]⁻ |
| | | (dd, J = 5.5, 2.5 Hz, 1H), 3.60 | |
| | | (t, J = 5.5 Hz, 2H), 3.57-3.55 | |
| | | (m, 2H), 3.58 (q, J = 5.5 Hz, | |
| | | 2H), 3.44-3.42 (m, 2H), 3.21 (s, | |
| | | 3H) ppm | |
| 25 | 2-(3,5-Dichloro-2-fluorophenyl)-5-((2- | ¹H NMR (500 MHz, DMSO-d₆) | ESI MS |
| | (2-hydroxyethoxy)ethyl)amino)oxazole- | δ 8.71 (s, 1H), 7.92 (dd, J = 6.0, | m/z 356 |
| | 4-carbonitrile | 2.5 Hz, 1H), 7.78 (dd, J = 6.0, | [M − H]⁻ |
| | | 2.5 Hz, 1H), 4.36 (t, J = 5.0 Hz, | |
| | | 1H), 3.40 (q, J = 6.0 Hz, 2H), | |
| | | 3.34 (t, J = 7.0 Hz, 2H), 1.60 | |
| | | (quint, J = 6.0 Hz, 2H), 1.44 | |
| | | (quint, J = 7.0 Hz, 2H), 1.40-1.35 | |
| | | (m, 2H) ppm | |
| 42 | 2-(2,5-Dichloro-3-(trifluoromethyl)phenyl)- | ¹H NMR (500 MHz, CDCl₃-d) | ESI MS |
| | 5-((2-methoxyethyl)amino)oxazole- | δ 8.04 (d, J = 2.6 Hz, 1H), 7.74 | m/z 380 |
| | 4-carbonitrile | (d, J = 2.6 Hz, 1H), 5.43 (t, J = | [M + H]⁺ |
| | | 5 Hz, 1H), 3.70-3.67 (m, 2H), | |
| | | 3.64-3.62 (m, 2H), 3.43 (s, 3H) ppm | |
| 37 | 2-(2,5-Dichloro-3-(trifluoromethyl)phenyl)- | ¹H NMR (500 MHz, CDCl₃-d) | ESI MS |
| | 5-((2-(2-methoxyethoxy)ethyl)amino)oxazole- | δ 8.03 (d, J = 2.6 Hz, 1H), 7.73 | m/z 424 |
| | 4-carbonitrile | (d, J = 2.6 Hz, 1H), 6.20 (t, J = | [M + H]⁺ |
| | | 5.5 Hz, 1H), 3.78-3.76 (m, | |
| | | 2H), 3.72-3.67 (m, 4H), 3.60-3.58 | |
| | | (m, 2H), 3.43 (s, 3H) ppm | |
| 20 | 2-(2,5-Dichloro-3-(trifluoromethyl)phenyl)- | ¹H NMR (300 MHz, DMSO-d₆) | ESI MS |
| | 5-((4-hydroxybutyl)amino)oxazole- | δ 8.77 (t, J = 5.6 Hz, 1H), 8.17 | m/z 394 |
| | 4-carbonitrile | (d, J = 2.5 Hz, 1H), 8.08 (d, J = | [M + H]⁺ |
| | | 2.5 Hz, 1H), 4.47 (t, J = 5.1 Hz, | |
| | | 1H), 3.46-3.36 (m, 4H), 1.68-1.59 | |
| | | (m, 2H), 1.54-1.47 (m, 2H) ppm | |
| 31 | 2-(2,5-Dichloro-3-(trifluoromethyl)phenyl)- | ¹H NMR (300 MHz, DMSO-d₆) | ESI MS |
| | 5-((2-(2-hydroxyethoxy)ethyl)amino)oxazole- | δ 8.81 (t, J = 5.6 Hz, 1H), 8.19 | m/z 410 |
| | 4-carbonitrile | (d, J = 2.5 Hz, 1H), 8.08 (d, J = | [M + H]⁺ |
| | | 2.5 Hz, 1H), 4.60 (t, J = 5.3 Hz, | |
| | | 1H), 3.63-3.60 (m, 2H), 3.56-3.47 | |
| | | (m, 6H) ppm | |
| 27 | 2-(2,5-Dichloro-3-(trifluoromethyl)phenyl)- | ¹H NMR (300 MHz, DMSO-d₆) | ESI MS |
| | 5-((5-hydroxypentyl)amino)oxazole- | δ 8.75 (t, J = 5.4 Hz, 1H), 8.16 | m/z 408 |
| | 4-carbonitrile | (d, J = 2.6 Hz, 1H), 8.07 (d, J = | [M + H]⁺ |
| | | 2.6 Hz, 1H), 4.38 (t, J = 5.1 Hz, | |
| | | 1H), 3.43-3.36 (m, 4H), 1.65-1.56 | |
| | | (m, 2H), 1.48-1.34 (m, 4H) ppm | |
| 36 | 2-(2,5-Dichloro-3-methylphenyl)-5-((2- | ¹H NMR (500 MHz, DMSO-d₆) | ESI MS |
| | (2-methoxyethoxy)ethyl)amino)oxazole- | δ 8.66 (t, J = 5.9 Hz, 1H), 7.71 | m/z 370 |
| | 4-carbonitrile | (dd, J = 2.6, 0.6 Hz, 1H), 7.63 | [M + H]⁺ |
| | | (dd, J = 2.6, 0.7 Hz, 1H), | |
| | | 3.62-3.57 (m, 2H), 3.57-3.53 (m, | |
| | | 2H), 3.52-3.46 (m, 2H), 3.44-3.41 | |
| | | (m, 2H), 3.21 (s, 3H), 2.40 | |
| | | (s, 3H) ppm | |
| 26 | 2-(2,5-Dichloro-3-methylphenyl)-5- | ¹H NMR (500 MHz, DMSO-d₆) | ESI MS |
| | ((5-hydroxypentyl)amino)oxazole- | δ 8.59 (t, J = 5.8 Hz, 1H), 7.69 | m/z 354 |
| | 4-carbonitrile | (dd, J = 2.6, 0.6 Hz, 1H), 7.63 | [M + H]⁺ |
| | | (dd, J = 2.6, 0.7 Hz, 1H), 4.35 | |
| | | (t, J = 5.1 Hz, 1H), 3.42-3.37 | |
| | | (m, 2H), 3.35-3.30 (m, 2H, | |
| | | overlaps with water), 3.39 (s, | |
| | | 3H), 1.63-1.56 (m, 2H), 1.48-1.41 | |
| | | (m, 2H), 1.40-1.32 (m, 2H) ppm | |
| 21 | 2-(2,3-Dichloro-5-methylphenyl)-5-((4- | ¹H NMR (300 MHz, CDCl₃) δ | ESI MS |
| | hydroxybutyl)amino)oxazole-4-carbonitrile | 7.58-7.57 (m, 1H), 7.37-7.36 | m/z 340 |
| | | (m, 1H), 5.64-5.68 (m, 1H), | [M + H]⁺ |
| | | 3.76 (t, J = 5.8 Hz, 2H), 3.54 (q, | |
| | | J = 6.0 Hz, 2H), 2.35 (s, 3H), | |
| | | 1.89-1.80 (m, 2H), 1.77-1.68 | |
| | | (m, 2H) ppm | |
| 38 | 2-(2,3-Dichloro-5-methylphenyl)-5-((2- | ¹H NMR (300 MHz, CDCl₃) δ | ESI MS |
| | (2-methoxyethoxy)ethyl)amino)oxazole- | 7.59-7.58 (m, 1H), 7.37-7.36 | m/z 370 |
| | 4-carbonitrile | (m, 1H), 5.84-5.79 (m, 1H), | [M + H]⁺ |
| | | 3.77-3.74 (m, 2H), 3.71-3.65 | |
| | | (m, 4H), 3.59-3.56 (m. 2H), | |
| | | 3.43 (s, 3H), 2.35 (s, 3H) ppm | |
| 23 | 2-(3,5-Dichloro-2-(difluoromethoxy)phenyl)- | ¹H NMR (300 MHz, CDCl₃) δ | ESI MS |
| | 5-((5-hydroxypentyl)amino)oxazole- | 7.83 (d, J = 2.5 Hz, 1H), 7.51 | m/z 406 |
| | 4-carbonitrile | (d, J = 2.5 Hz, 1H), 6.68 (t, J = | [M + H]⁺ |
| | | 74.9 Hz, 1H), 5.33 (t, J = 5.7 | |
| | | Hz, 1H), 3.70 (q, J = 5.9 Hz, | |
| | | 2H), 3.52 (q, J = 6.8 Hz, 2H), | |
| | | 1.80-1.70 (m, 2H), 1.70-1.61 | |
| | | (m, 2H), 1.56-1.50 (m, 2H) ppm | |
| 18 | 2-(3,5-Dichloro-2-(difluoromethoxy)phenyl)- | ¹H NMR (300 MHz, CDCl₃) δ | ESI MS |
| | 5-((4-hydroxybutyl)amino)oxazole- | 7.83 (d, J = 2.5 Hz, 1H), 7.50 | m/z 392 |
| | 4-carbonitrile | (d, J = 2.5 Hz, 1H), 6.68 (t, J = | [M + H]⁺ |
| | | 74.9 Hz, 1H), 5.96-5.92 (m, | |
| | | 1H), 3.76 (q, J = 5.7 Hz, 2H), | |
| | | 3.57-3.51 (m, 2H), 1.89-1.67 | |
| | | (m, 4H) ppm | |
| 29 | 2-(3,5-Dichloro-2-(difluoromethoxy)phenyl)- | ¹H NMR (300 MHz, CDCl₃) δ | ESI MS |
| | 5-((2-(2-hydroxyethoxy)ethyl)amino)oxazole- | 7.84 (d, J = 2.5 Hz, 1H), 7.51 | m/z 406 |
| | 4-carbonitrile | (d, J = 2.5 Hz, 1H), 6.68 (t, J = | [M − H]⁻ |
| | | 74.9 Hz, 1H), 6.03 (t, J = 5.5 | |
| | | Hz, 1H), 3.83-3.65 (m, 8H) ppm | |
| 33 | 2-(3,5-Dichloro-2-(difluoromethoxy)phenyl)- | ¹H NMR (300 MHz, CDCl₃) δ | ESI MS |
| | 5-((2-(2-methoxyethoxy)ethyl)amino)oxazole- | 7.83 (d, J = 2.5 Hz, 1H), 7.50 | m/z 422 |
| | 4-carbonitrile | (d, J = 2.5 Hz, 1H), 6.68 (t, J = | [M + H]⁺ |
| | | 74.9 Hz, 1H), 6.11 (t, J = 5.3 | |
| | | Hz, 1H), 3.76-3.65 (m, 6H), | |
| | | 3.59-3.56 (m, 2H), 3.43 (s, 3H) ppm | |
| 40 | 2-(3,5-Dichloro-2-(difluoromethoxy)phenyl)- | ¹H NMR (300 MHz, CDCl₃) δ | ESI MS |
| | 5-((2-methoxyethyl)amino)oxazole- | 7.83 (d, J = 2.5 Hz, 1H), 7.51 (d, | m/z 378 |
| | 4-carbonitrile | J = 2.5 Hz, 1H), 6.68 (t, J = | [M + H]⁺ |
| | | 74.9 Hz, 1H), 5.41-5.37 (m, | |
| | | 1H), 3.70-3.60 (m, 4H), 3.42 (s, | |
| | | 3H) ppm | |
| 72 | 5-((2-(2-(3,3-Difluoroazetidin-1- | ¹H NMR (500 MHz, DMSO-d₆) | ESI MS |
| | yl)ethoxy)ethyl)amino)-2-(2,3,5- | δ 8.74 (t, J = 5.8 Hz, 1H), 7.99 | m/z 451 |
| | trichlorophenyl)oxazole-4-carbonitrile | (d, J = 2.5 Hz, 1H), 7.87 (d, J = | [M + H]⁺ |
| | | 2.5 Hz, 1H), 3.58-3.49 (m, 8H), | |
| | | 3.46 (t, J = 5.4 Hz, 2H), 2.66 (t, | |
| | | J = 5.4 Hz, 2H) ppm | |
| 73 | 5-((2-(2-(Methyl(2,2,2- | ¹H NMR (500 MHz, DMSO-d₆) | ESI MS |
| | trifluoroethyl)amino)ethoxy)ethyl)amino)- | δ 8.74 (t, J = 5.8 Hz, 1H), 7.99 | m/z 469 |
| | 2-(2,3,5-trichlorophenyl)oxazole- | (d, J = 2.5 Hz, 1H), 7.86 (d, J = | [M − H]⁻ |
| | 4-carbonitrile | 2.5 Hz, 1H), 3.60-3.56 (m, 2H), | |
| | | 3.55-3.49 (m, 4H), 3.18 (q, J = | |
| | | 10.2 Hz, 2H), 2.71 (t, J = 5.6 | |
| | | Hz, 2H), 2.35 (s, 3H) ppm | |
| 74 | 5-((2-(2-(3-Fluoropiperidin-1- | ¹H NMR (500 MHz, DMSO-d₆) | ESI MS |
| | yl)ethoxy)ethyl)amino)-2- | δ 8.73 (t, J = 5.7 Hz, 1H), 7.99 | m/z 461 |
| | (2,3,5-trichlorophenyl)oxazole- | (d, J = 2.5 Hz, 1H), 7.87 (d, J = | [M + H]⁺ |
| | 4-carbonitrile | 2.5 Hz, 1H), 4.62-4.42 (m, 1H), | |
| | | 3.60-3.56 (m, 2H), 3.55-3.48 | |
| | | (m, 4H), 2.76-2.62 (m, 1H), | |
| | | 2.50-2.45 (m, 1H, overlaps with | |
| | | solvent), 2.44-2.24 (m, 3H), | |
| | | 2.23-2.12 (m, 1H), 1.82-1.54 | |
| | | (m, 2H), 1.49-1.29 (m, 2H) ppm | |
| 75 | 5-((2-(2-(3,3-Difluoropiperidin-1- | ¹H NMR (500 MHz, DMSO-d₆) | ESI MS |
| | yl)ethoxy)ethyl)amino)-2-(2,3,5- | δ 8.73 (t, J = 5.8 Hz, 1H), 7.99 | m/z 479 |
| | trichlorophenyl)oxazole-4-carbonitrile | (d, J = 2.5 Hz, 1H), 7.87 (d, J = | [M + H]⁺ |
| | | 2.5 Hz, 1H), 3.61-3.47 (m, 6H), | |
| | | 2.63 (t, J = 11.8 Hz, 2H), 2.55 | |
| | | (t, J = 5.7 Hz, 2H), 2.41-2.37 | |
| | | (m, 2H), 1.84-1.73 (m, 2H), | |
| | | 1.60-1.52 (m, 2H) ppm | |
| 76 | 5-((2-(2-(Pyridin-3- | ¹H NMR (500 MHz, DMSO-d₆) | ESI MS |
| | yloxy)ethoxy)ethyl)amino)-2-(2,3,5- | δ 8.77 (t, J = 5.9 Hz, 1H), 8.23 | m/z 453 |
| | trichlorophenyl)oxazole-4-carbonitrile | (d, J = 2.7 Hz, 1H), 8.12 (dd, J = | [M + H]⁺ |
| | | 4.6, 1.4 Hz, 1H), 7.96 (d, J = | |
| | | 2.5 Hz, 1H), 7.84 (d, J = 2.5 Hz, | |
| | | 1H), 7.34-7.30 (m, 1H), 7.29-7.25 | |
| | | (m, 1H), 4.17-4.13 (m, | |
| | | 2H), 3.81-3.78 (m, 2H), 3.68 (t, | |
| | | J = 5.4 Hz, 2H), 3.57-3.52 (m, | |
| | | 2H) ppm | |
| 77 | 5-((2-(2-(3-Fluoroazetidin-1- | ¹H NMR (500 MHz, DMSO-d₆) | ESI MS |
| | yl)ethoxy)ethyl)amino)-2-(2,3,5- | δ 8.76 (t, J = 5.7 Hz, 1H), 7.99 | m/z 433 |
| | trichlorophenyl)oxazole-4-carbonitrile | (d, J = 2.5 Hz, 1H), 7.87 (d, J = | [M + H]⁺ |
| | | 2.5 Hz, 1H), 5.14-4.95 (m, 1H), | |
| | | 3.57-3.46 (m, 6H), 3.42 (t, J = | |
| | | 5.5 Hz, 2H), 2.57 (t, J = 5.5 Hz, | |
| | | 2H), 2.19-2.14 (m, 1H), 1.93-1.86 | |
| | | (m, 1H) ppm | |
| 78 | 5-((2-(2-(3-Fluoropyrrolidin- | ¹H NMR (500 MHz, DMSO-d₆) | ESI MS |
| | 1-yl)ethoxy)ethyl)amino)-2-(2,3,5- | δ 8.76 (t, J = 5.7 Hz, 1H), 7.99 | m/z 447 |
| | trichlorophenyl)oxazole-4-carbonitrile | (d, J = 2.5 Hz, 1H), 7.87 (d, J = | [M + H]⁺ |
| | | 2.5 Hz, 1H), 5.18-5.01 (m, 1H), | |
| | | 3.60-3.56 (m, 2H), 3.55-3.50 | |
| | | (m, 4H), 2.83-2.69 (m, 2H), | |
| | | 2.62-2.52 (m, 3H), 2.31-2.22 | |
| | | (m, 1H), 2.11-1.95 (m, 1H), | |
| | | 1.85-1.68 (m, 1H) ppm | |
| 79 | 5-((2-(2-(Pyridazin-3- | ¹H NMR (500 MHz, DMSO-d₆) | ESI MS |
| | yloxy)ethoxy)ethyl)amino)-2-(2,3,5- | δ 8.67 (t, J = 5.7 Hz, 1H), 7.98 | m/z 454 |
| | trichlorophenyl)oxazole-4-carbonitrile | (d, J = 2.5 Hz, 1H), 7.86 (d, J = | [M + H]⁺ |
| | | 2.5 Hz, 1H), 7.82 (dd, J = 3.8, | |
| | | 1.7 Hz, 1H), 7.35 (dd, J = 9.4, | |
| | | 3.8, 1H), 6.86 (dd, J = 9.4, 1.7 | |
| | | Hz, 1H), 4.22 (t, J = 5.7 Hz, | |
| | | 2H), 3.80 (t, J = 5.7 Hz, 2H), | |
| | | 3.61 (t, J = 5.4 Hz, 2H), 3.50-3.44 | |
| | | (m, 2H) ppm | |
| 80 | 5-((2-(2-(4-Fluoropiperidin-1- | ¹H NMR (500 MHz, DMSO-d₆) | ESI MS |
| | yl)ethoxy)ethyl)amino)-2-(2,3,5- | δ 8.74 (t, J = 5.6 Hz, 1H), 8.00 | m/z 461 |
| | trichlorophenyl)oxazole-4-carbonitrile | (d, J = 2.5 Hz, 1H), 7.9 (d, J = | [M + H]⁺ |
| | | 2.5 Hz, 1H), 4.65-4.52 (m, 1H), | |
| | | 3.59-3.51 (m, 6H), 2.44 (t, J = | |
| | | 5.8 Hz, 2H), 2.27-2.24 (m, 2H), | |
| | | 1.81-1.71 (m, 2H), 1.65-1.57 (m, | |
| | | 2H) ppm | |
| 81 | 5-((2-(2-(Phenylamino)ethoxy)ethyl)amino)- | ¹H NMR (500 MHz, DMSO-d₆) | ESI MS |
| | 2-(2,3,5-trichlorophenyl)oxazole- | δ 8.77 (t, J = 5.8 Hz, 1H), 7.98 | m/z 451 |
| | 4-carbonitrile | (d, J = 2.5 Hz, 1H), 7.86 (d, J = | [M + H]⁺ |
| | | 2.5 Hz, 1H), 7.05-7.01 (m, 2H), | |
| | | 6.56-6.54 (m, 2H), 6.51-6.48 | |
| | | (m, 1H), 5.42 (t, J = 5.7 Hz, | |
| | | 1H), 3.65-3.60 (m, 4H), 3.56-3.53 | |
| | | (m, 2H), 3.17 (q, J = 5.8 Hz, 2H) ppm | |
| 82 | 5-((2-(2-(Pyrimidin-5- | ¹H NMR (500 MHz, DMSO-d₆) | ESI MS |
| | yloxy)ethoxy)ethyl)amino)-2-(2,3,5- | δ 8.78-8.76 (m, 2H), 8.50 (s, | m/z 452 |
| | trichlorophenyl)oxazole-4-carbonitrile | 2H), 7.96 (d, J = 2.5 Hz, 1H), | [M − H]⁻ |
| | | 7.84 (d, J = 2.5 Hz, 1H), 4.27-4.26 | |
| | | (m, 2H), 3.82-3.80 (m, | |
| | | 2H), 3.68 (t, J = 5.4 Hz, 2H), | |
| | | 3.5 (q, J = 5.6 Hz, 2H) ppm | |
| 83 | 5-((2-(2-((2,6-Dimethylpyridin-4- | ¹H NMR (500 MHz, DMSO-d₆) | ESI MS |
| | yl)oxy)ethoxy)ethyl)amino)-2-(2,3,5- | δ 8.76 (t, J = 5.9 Hz, 1H), 7.95 | m/z 480 |
| | trichlorophenyl)oxazole-4-carbonitrile | (d, J = 2.5 Hz, 1H), 7.82 (d, J = | [M]⁺ |
| | | 2.5 Hz, 1H), 6.53 (s, 2H), 4.11-4.09 | |
| | | (m, 2H), 3.78-3.76 (m, 2H), | |
| | | 3.67 (t, J = 5.3 Hz, 2H), 3.54 | |
| | | (q, J = 5.6 Hz, 2H), 2.30 (s, 6H) ppm | |
| 84 | 5-((2-(2-((3-Fluoropyridin-4- | ¹H NMR (500 MHz, DMSO-d₆) | ESI MS |
| | yl)oxy)ethoxy)ethyl)amino)-2-(2,3,5- | δ 8.77 (t, J = 5.8 Hz, 1H), 8.35 | m/z 470 |
| | trichlorophenyl)oxazole-4-carbonitrile | (d, J = 3.4 Hz, 1H), 8.23 (d, J = | [M]⁺ |
| | | 5.6 Hz, 1H), 7.95 (d, J = 2.5 | |
| | | Hz, 1H), 7.83 (d, J = 2.5 Hz, | |
| | | 1H), 7.21 (q, J = 5.6 Hz, 2 Hz, | |
| | | 1H), 4.28-4.26 (m, 2H), 3.84-3.83 | |
| | | (m, 2H), 3.69 (t, J = 5.4 Hz, | |
| | | 2H), 3.55 (q, J = 5.4 Hz, 2H) ppm | |
| 103 | 5-((2-(2-(Pyridazin-4- | ¹H NMR (300 MHz, CDCl₃) δ | ESI MS |
| | yloxy)ethoxy)ethyl)amino)-2-(2,3,5- | 7.90 (d, J = 2.7 Hz, 1H), 7.79 | m/z 454 |
| | trichlorophenyl)oxazole-4-carbonitrile | (d, J = 7.7 Hz, 1H), 7.76 (d, J = | [M + H]⁺ |
| | | 2.5 Hz, 1H), 7.54 (d, J = 2.4 | |
| | | Hz, 1H), 6.41 (dd, J = 7.8, 3.1 | |
| | | Hz, 1H), 6.01-5.98 (m, 1H), | |
| | | 4.20 (t, J = 4.5 Hz, 2H), 3.88 (t, | |
| | | J = 5.0 Hz, 2H), 3.71-3.66 (m, 4H) ppm | |
| 102 | 5-((2-(2-((2-Fluoropyridin-4- | ¹H NMR (300 MHz, CDCl₃) δ | ESI MS |
| | yl)oxy)ethoxy)ethyl)amino)-2-(2,3,5- | 8.04 (d, J = 5.8 Hz, 1H), 7.76 | m/z 471 |
| | trichlorophenyl)oxazole-4-carbonitrile | (d, J = 2.4 Hz, 1H), 7.54 (d, J = | [M + H]⁺ |
| | | 2.4 Hz, 1H), 6.75-6.72 (m, 1H), | |
| | | 6.40 (d, J = 2.1 Hz, 1H), 5.46 (t, | |
| | | J = 5.4 Hz, 1H), 4.22-4.19 (m, | |
| | | 2H), 3.92-3.89 (m, 2H), 3.82-3.79 | |
| | | (m, 2H), 3.74-3.69 (m, 2H) ppm | |
| 101 | 5-((2-(2-((2-Methoxypyridin-4- | ¹H NMR (300 MHz, CDCl₃) δ | ESI MS |
| | yl)oxy)ethoxy)ethyl)amino)-2-(2,3,5- | 7.97 (d, J = 5.9 Hz, 1H), 7.76 | m/z 481 |
| | trichlorophenyl)oxazole-4-carbonitrile | (d, J = 2.4 Hz, 1H), 7.54 (d, J = | [M − H]⁻ |
| | | 2.4 Hz, 1H), 6.49 (dd, J = 5.9, | |
| | | 2.4 Hz, 1H), 6.20 (d, J = 2.1 | |
| | | Hz, 1H), 5.43 (t, J = 6.2 Hz, | |
| | | 1H), 4.17-4.14 (m, 2H), 3.91 (s, | |
| | | 3H), 3.89-3.87 (m, 2H), 3.82-3.78 | |
| | | (m, 2H), 3.74-3.69 (m, 2H) ppm | |
| 100 | 5-((2-(2-(2-(Pyridin-4- | ¹H NMR (300 MHz, CDCl₃) δ | ESI MS |
| | yloxy)ethoxy)ethoxy)ethyl)amino)-2-(2,3,5- | 8.42-8.39 (m, 2H), 7.75 (d, J = | m/z 497 |
| | trichlorophenyl)oxazole-4-carbonitrile | 3.0 Hz, 1H), 7.53 (d, J = 2.4 | [M + H]⁺ |
| | | Hz, 1H), 6.82-6.79 (m, 2H), | |
| | | 5.80-5.76 (m, 1H), 4.23-4.19 | |
| | | (m, 2H), 3.91 (t, J = 4.7 Hz, | |
| | | 2H), 3.77-3.71 (m, 6H), 3.70-3.66 | |
| | | (m, 2H) ppm | |
| 99 | 5-((2-(2-((3-Methoxypyridin-4- | ¹H NMR (300 MHz, CDCl₃) δ | ESI MS |
| | yl)oxy)ethoxy)ethyl)amino)-2-(2,3,5- | 8.18 (t, J = 2.2 Hz, 2H), 7.75 (d, | m/z 481 |
| | trichlorophenyl)oxazole-4-carbonitrile | J = 2.4 Hz, 1H), 7.53 (d, J = 2.4 | [M − H]⁻ |
| | | Hz, 1H), 6.80 (d, J = 5.3 Hz, | |
| | | 1H), 5.90-5.85 (m, 1H), 4.24-4.21 | |
| | | (m, 2H), 3.97-3.95 (m, 2H), | |
| | | 3.94 (s, 3H), 3.85-3.82 (m, | |
| | | 2H), 3.74-3.71 (m, 2H) ppm | |
| 98 | 5-((2-(2-(4,4-Difluoropiperidin- | ¹H NMR (300 MHz, CDCl₃) δ | ESI MS |
| | 1-yl)ethoxy)ethyl)amino)-2-(2,3,5- | 7.74 (d, J = 2.4 Hz, 1H), 7.54 | m/z 479 |
| | trichlorophenyl)oxazole-4-carbonitrile | (d, J = 2.4 Hz, 1H), 6.53-6.38 | [M + H]⁺ |
| | | (m, 1H), 3.74-3.65 (m, 6H), | |
| | | 2.76-2.60 (m, 6H), 2.15-1.97 | |
| | | (m, 4H) ppm | |
| 97 | 5-((2-(2-(Pyridin-4- | ¹H NMR (300 MHz, CDCl₃) δ | ESI MS |
| | yloxy)ethoxy)ethyl)amino)-2-(2,3,5- | 8.44 (d, J = 6.1 Hz, 2H), 7.76 | m/z 453 |
| | trichlorophenyl)oxazole-4-carbonitrile | (d, J = 2.4 Hz, 1H), 7.54 (d, J = | [M + H]⁺ |
| | | 2.4 Hz, 1H), 6.82 (d, J = 6.1 | |
| | | Hz, 2H), 5.58 (s, 1H), 4.20-4.17 | |
| | | (m, 2H), 3.92-3.89 (m, 2H), | |
| | | 3.82-3.79 (m, 2H), 3.75-3.69 | |
| | | (m, 2H) ppm | |
| 96 | 5-((2-(2-((2- | ¹H NMR (500 MHz, DMSO-d₆) | ESI MS |
| | Fluoroethyl)(methyl)amino)ethoxy)ethyl)amino)- | δ 8.76 (t, J = 5 Hz, 1H), 7.99 (d, | m/z 435 |
| | 2-(2,3,5-trichlorophenyl)oxazole-4-carbonitrile | J = 2.5 Hz, 1H), 7.87 (d, J = 2.5 | [M + H]⁺ |
| | | Hz, 1H), 4.44 (dt, J = 42.5, 5 | |
| | | Hz, 2H), 3.58 (t, J = 5.5 Hz, | |
| | | 2H), 3.52 (t, J = 5.5 Hz, 4H), | |
| | | 2.63 (dt, J = 27.5, 5.0 Hz, 2 H), | |
| | | 2.54 (t, J = 6.0 Hz, 2H), 2.20 (s, | |
| | | 3H) ppm | |
| 95 | 5-((2-(2- | ¹H NMR (500 MHz, DMSO-d₆) | ESI MS |
| | (Methyl(phenyl)amino)ethoxy)ethyl)amino)-2- | δ 8.72 (s, 1H), 7.98 (d, J = 2.5 | m/z 465 |
| | (2,3,5-trichlorophenyl)oxazole-4-carbonitrile | Hz, 1H), 7.84 (d, J = 2.5 Hz, | [M + H]⁺ |
| | | 1H), 7.11-7.08 (m, 2H), 6.64 (d, | |
| | | J = 8.0 Hz, 2H), 6.54 (t, J = 7.0 | |
| | | Hz, 1H), 3.59 (t, J = 6.0 Hz, | |
| | | 4H), 3.51 (t, J = 5.5 Hz, 2H), | |
| | | 3.45 (t, J = 6.0 Hz, 2H), 2.84 (s, | |
| | | 3H) ppm | |
| 94 | 2-(2,3,5-Trichlorophenyl)-5-((2-(2-((2,2,2- | ¹H NMR (500 MHz, DMSO-d₆) | ESI MS |
| | trifluoroethyl)amino)ethoxy)ethyl)amino)oxazole- | δ 8.75 (t, J = 5.5 Hz, 1H), 7.99 | m/z 456 |
| | 4-carbonitrile | (d, J = 2.0 Hz, 1H), 7.86 (d, J = | [M − H]⁻ |
| | | 2.5 Hz, 1H), 3.59 (t, J = 5.0 Hz, | |
| | | 2H), 3.53 (q, J = 5.5 Hz, 2H), | |
| | | 3.49 (t, J = 6.0 Hz, 2H), 3.24-3.21 | |
| | | (m, 2H), 2.75 (d, J = 4.0 | |
| | | Hz, 2H), 2.29 (br s, 1H) ppm | |
| 93 | 2-(2,3,5-Trichlorophenyl)-5-((2-(2-((2,2,2- | ¹H NMR (500 MHz, DMSO-d₆) | ESI MS |
| | trifluoroethyl)amino)ethoxy)ethyl)amino)oxazole- | δ 7.99 (d, J = 2.5 Hz, 1H), 7.87 | m/z 421 |
| | 4-carbonitrile | (d, J = 2.0 Hz, 1H), 4.41 (dt, J = | [M + H]⁺ |
| | | 47.5, 5.0 Hz, 2H), 3.60 (t, J = | |
| | | 5.0 Hz, 2H), 3.54-3.45 (m, 4H), | |
| | | 2.77 (dt, J = 27.5, 5.0 Hz, 2H), | |
| | | 2.68 (t, J = 5.5 Hz, 2H), ppm | |
| 92 | 5-((2-(2-(3,3-Difluoropyrrolidin-1- | ¹H NMR (500 MHz, DMSO-d₆) | ESI MS |
| | yl)ethoxy)ethyl)amino)-2-(2,3,5- | δ 8.72 (br s, 1H), 7.81 (d, J = | m/z 465 |
| | trichlorophenyl)oxazole-4-carbonitrile | 2.5 Hz, 1H), 7.77 (d, J = 2.5 | [M + H]⁺ |
| | | Hz, 1H), 3.75-3.49 (m, 6H), | |
| | | 2.86 (t, J = 22.0 Hz, 2H) 2.67 | |
| | | (t, J = 7.0 Hz, 2H), 2.58 (t, J = | |
| | | 5.5 Hz, 2H), 2.14 (septuplet, J = | |
| | | 7.0 Hz, 2H) ppm | |
| 91 | 5-((2-(2-(Pyridin-4-ylamino)ethoxy)ethyl)amino)- | ¹H NMR (500 MHz, DMSO-d₆) | ESI MS |
| | 2-(2,3,5-trichlorophenyl)oxazole-4-carbonitrile | δ 8.69 (t, J = 5.5 Hz, 1H), 8.07 | m/z 452 |
| | | (d, J = 7.0 Hz, 2H), 8.043 (s, | [M + H]⁺ |
| | | 2H), 8.00 (d, J = 2.5 Hz, 1H), | |
| | | 7.86 (d, J = 2.5 Hz, 1H), 6.79 | |
| | | (d, J = 6.5 Hz, 2H), 6.51 (br s, | |
| | | 1H), 4.27 (t, J = 5.0 Hz, 2H), | |
| | | 3.79 (t, J = 5.0 Hz, 2H), 3.60 (t, | |
| | | J = 5.5 Hz, 2H), 3.50 (q, J = 5.5 | |
| | | Hz, 2H) ppm | |
| 90 | 5-((2-(2-(Methyl(pyridin-4- | ¹H NMR (500 MHz, DMSO-d₆) | ESI MS |
| | yl)amino)ethoxy)ethyl)amino)-2-(2,3,5- | δ 8.67 (t, J = 6.0 Hz, 1H), 8.61 | m/z 466 |
| | trichlorophenyl)oxazole-4-carbonitrile | (q, J = 4.5 Hz, 1H), 8.20 (d, J = | [M + H]⁺ |
| | | 6.0 Hz, 1H), 8.03-7.99 (m, 2H), | |
| | | 7.85 (d, J = 2.5 Hz, 1H), 6.76-6.74 | |
| | | (m, 2H), 6.52 (s, 1H), 4.29 | |
| | | (t, J = 5.0 Hz, 2), 3.77 (t, J = | |
| | | 5.0 Hz, 2H), 3.60 (t, J = 5.0 Hz, | |
| | | 2H), 3.49 (q, J = 5.5 Hz, 2H), | |
| | | 2.84 (d, J = 5.0 Hz, 3H) ppm | |
| 43 | 5-((4-(Benzyloxy)but-2-yn-1-yl)amino)-2-(2,3,5- | ¹H NMR (300 MHz, DMSO-d₆) | ESI MS |
| | trichlorophenyl)oxazole-4-carbonitrile | δ 9.18 (t, J = 6.3 Hz, 1H), 8.00 | m/z 444 |
| | | (d, J = 2.5 Hz, 1H), 7.91 (d, J = | [M − H]⁻ |
| | | 2.5 Hz, 1H), 7.34-7.23 (m, 5H), | |
| | | 4.49 (s, 2H), 4.30 (d, J = 5.9 | |
| | | Hz, 2H), 4.21 (s, 2H) ppm | |
| 89 | 4-(((4-((4-Cyano-2-(2,3,5- | ¹H NMR (500 MHz, DMSO-d₆) | ESI MS |
| | trichlorophenyl)oxazol-5-yl)amino)but- | δ 12.84 (br s, 1H), 9.15 (t, J = | m/z 488 |
| | 2-yn-1-yl)oxy)methyl)benzoic acid | 5.8 Hz, 1H), 7.97 (d, J = 2.5 | [M − H]⁻ |
| | | Hz, 1H), 7.89 (d, J = 2.5 Hz, | |
| | | 1H), 7.88-7.85 (m, 2H), 7.37-7.34 | |
| | | (m, 2H), 4.57 (s, 2H), | |
| | | 4.31-4.25 (m, 4H) ppm | |
| 88 | 5-((4-(Pyridin-4-ylmethoxy)but-2-yn-1-yl)amino)- | ¹H NMR (300 MHz, CDCl₃) δ | ESI MS |
| | 2-(2,3,5-trichlorophenyl)oxazole-4-carbonitrile | 8.61-8.55 (m, 2H), 7.79 (d, J = | m/z 447 |
| | | 2.4 Hz, 1H), 7.55 (d, J = 2.4 | [M + H]⁺ |
| | | Hz, 1H), 7.26-7.23 (m, 2H), | |
| | | 5.61-5.57 (m, 1H), 4.60 (s, 2H), | |
| | | 4.34-4.27 (m, 4H) ppm | |
| 87 | 5-((4-(Pyridin-4-ylmethoxy)but-2-yn-1-yl)amino)- | ¹H NMR (300 MHz, CDCl₃) δ | ESI MS |
| | 2-(2,3,5-trichlorophenyl)oxazole-4-carbonitrile | 7.78 (d, J = 2.4 Hz, 1H), 7.55(d, | m/z 575 |
| | | J = 2.4 Hz, 1H), 7.26-7.23 (m, | [M + H]⁺ |
| | | 2H), 6.88-6.85 (m, 2H), 5.35-5.30 | |
| | | (m, 1H), 4.50 (s, 2H), | |
| | | 4.33-4.29 (m, 2H), 4.17-4.08 | |
| | | (m, 4H), 3.73 (t, J = 4.6 Hz, | |
| | | 4H), 2.80 (t, J = 5.7 Hz, 2H), | |
| | | 2.58 (t, J = 4.6 Hz, 4H) ppm | |
| 86 | 5-((4-((4-(2- | ¹H NMR (300 MHz, CDCl₃) δ | ESI MS |
| | Morpholinoethoxy)benzyl)oxy)but-2-yn-1-yl)amino)- | 7.78 (d, J = 2.4 Hz, 1H), 7.55 | m/z 559 |
| | 2-(2,3,5-trichlorophenyl)oxazole-4-carbonitrile | (d, J = 2.4 Hz, 1H), 7.26-7.23 | [M + H]⁺ |
| | | (m, 2H), 7.19-7.16 (m, 2H), | |
| | | 5.31 (t, J = 6.1 Hz, 1H), 4.54 (s, | |
| | | 2H), 4.32-4.29 (m, 2H), 4.19 (t, | |
| | | J = 1.7 Hz, 2H), 3.74 (t, J = 4.5 | |
| | | Hz, 4H), 2.82-2.78 (m, 2H), | |
| | | 2.60-2.50 (m, 6H) ppm | |
| 85 | 5-((4-((4-(2- | ¹H NMR (300 MHz, CDCl₃) δ | ESI MS |
| | Morpholinoethyl)benzyl)oxy)but-2-yn-1-yl)amino)- | 7.78 (d, J = 2.4 Hz, 1H), 7.55 | m/z 518 |
| | 2-(2,3,5-trichlorophenyl)oxazole-4-carbonitrile | (d, J = 2.4 Hz, 1H), 7.26-7.22 | [M − H]⁻ |
| | | (m, 2H), 6.92-6.88 (m, 2H), | |
| | | 5.32 (t, J =5.9 Hz, 1H), 4.50 (s, | |
| | | 2H), 4.32-4.29 (m, 2H), 4.16-4.09 | |
| | | (m, 4H), 3.76-3.73 (m, | |
| | | 2H), 3.45 (s, 3H) ppm | |

### Example 2—Biological Activity of Exemplified Compounds as Inhibitors of 12/15-LOX

| | | h12/15- | |
|---|---|---|---|
| | | LOX | Max % |
| Cmpd. | | IC₅₀, | inhi- |
| no. | Cmpd. structure | μM | bition |
| | 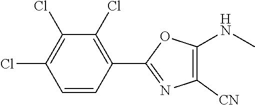 [SMILES: CNc1oc(-c2ccc(Cl)c(Cl)c2Cl)nc1C#N] | >20 | 49 |
| | 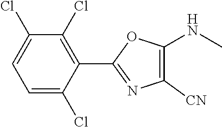 [SMILES: CNc1oc(-c2c(Cl)ccc(Cl)c2Cl)nc1C#N] | >20 | 32 |
| | 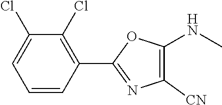 [SMILES: CNc1oc(-c2cccc(Cl)c2Cl)nc1C#N] | 0.67 | 69 |

### Example 2A—Analytical Data for the Compounds of Example 2

| Cmpd. | | | |
|---|---|---|---|
| No. | Name | NMR | Mass spec |
| 31081 | 5-(methylamino)-2-(2,3,4- | ¹H NMR (300 MHz, | ESI MS m/z |
| | trichlorophenyl)oxazole-4- | DMSO-d₆) δ 8.59 (s, 1H), | 300 [M − H]⁻. |
| | carbonitrile | 7.82 (q, J = 8.7 Hz, 7.4 Hz, | |
| | | 2H), 2.99 (s, 3H) ppm | |
| 31130 | 5-(methylamino)-2-(2,3,6- | ¹H NMR (300 MHz, | ESI MS m/z |
| | trichlorophenyl)oxazole-4- | CDCl₃) δ 7.54 (d, J = 8.7 | 301 [M + H]⁺. |
| | carbonitrile | Hz, 1H), 7.36 (d, J = 8.7 Hz, | |
| | | 1H), 4.93-4.92 (m, 1H), | |
| | | 3.18 (d, J = 5.3 Hz, 3H) ppm | |

### Example 3—Aqueous Solubility of Exemplified Compounds

| Cmpd. no. | Sol, μM |
|---|---|
| 1 | + |
| 45 | + |
| 46 | + |
| 47 | + |
| 43 | + |
| 16 | +++ |
| 17 | ++ |
| 22 | + |
| 48 | + |
| 39 | + |
| 28 | +++ |
| 32 | ++ |
| 49 | + |
| 50 | +++ |

“+” < 10 μM
“++” 10-20 μM
“+++” 20-50 μM

Combinations of Solutol HS-15 (also known as Kolliphor HS-15) with PEG400 provided a good vehicle to solubilize the tested compounds. Specifically, 10% Solutol/90% PEG400 was used to prepare 20 mg/ml solutions of compound 1, compound 44, and compound 32. 10% Solutol/15% H₂O/90% PEG400 led to 10 mg/ml solutions of the tested compounds.

### Example 4—Metabolic Stability of Exemplified Compounds

| | Human liver | | Rat liver | | Mouse liver | |
|---|---|---|---|---|---|---|
| | microsomes | | microsomes | | microsomes | |
| | | Extr | | Extr | | Extr |
| Cmpd. | CLᵢₙₜ, | (E_{H}), | CLᵢₙₜ, | (E_{H}), | CLᵢₙₜ, | (E_{H}), |
| no. | μL/min/mg | % | μL/min/mg | % | μL/min/mg | % |
| 1 | ++ | ** | + | * | ++ | ** |
| 45 | ++ | ** | ++ | ** | +++ | ** |
| 46 | ++ | ** | ++ | ** | +++ | ** |
| 47 | +++ | ** | +++ | ** | +++ | ** |
| 43 | +++ | ** | ++ | ** | ++ | ** |
| 16 | + | * | ++ | * | +++ | ** |
| 17 | + | * | + | * | + | * |
| 22 | ++ | ** | ++ | * | + | * |
| 48 | ++ | ** | ++ | ** | +++ | ** |
| 39 | ++ | ** | ++ | ** | +++ | ** |
| 28 | ++ | ** | + | * | ++ | * |
| 32 | + | * | + | * | + | * |
| 49 | + | * | ++ | ** | ++ | ** |
| 50 | ++ | ** | ++ | ** | ++ | ** |

“+” <20 μL/min/mg
“++” 20-100 μL/min/mg
“+++” 100-200 μL/min/mg
“*” <50%
“**” 50-100%

### Example 5—Biological Activity of Exemplified Compounds as Prodrugs

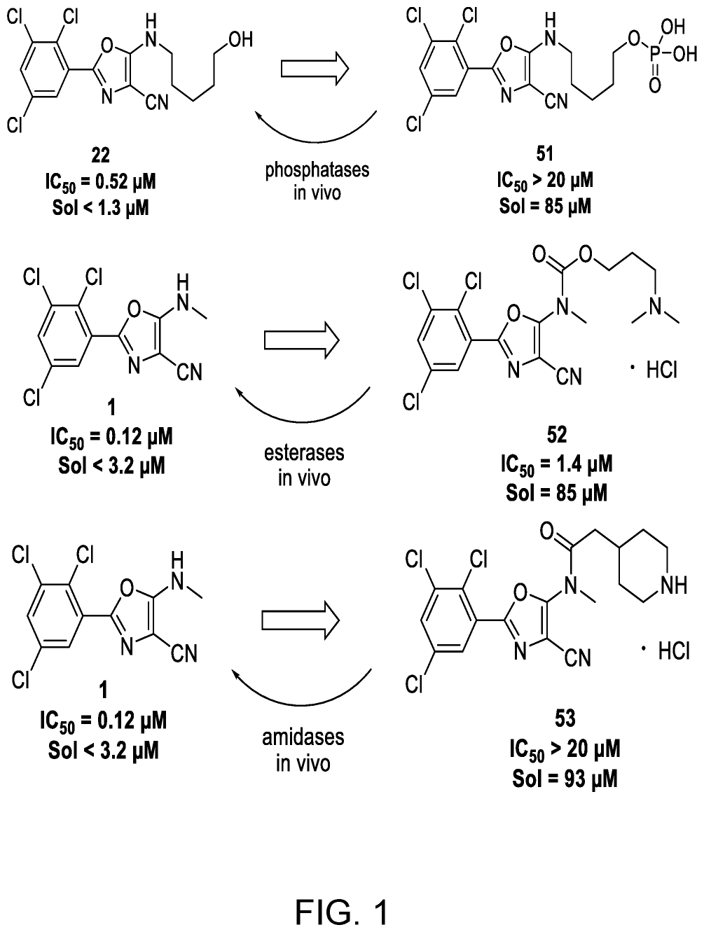

Compound 51 demonstrated high aqueous solubility versus parent compound 22, and was susceptible to hydrolysis in plasma. Compound 52 surprisingly showed activity against human 12/15-LOX in vitro, with an IC₅₀=1.4 μM. Compound 52 shows great solubility in Captisol, with 27 mg/ml achieved in a 50% Captisol solution. 20 mg/ml were obtained in 20% Captisol, allowing for intravenous delivery in the mouse stroke model. In vivo, compound 52 is processed to the parent molecule, compound 1 (see FIG. 1).

### Example 6—Evaluation of Exemplified Compounds in Animal Model of Stroke

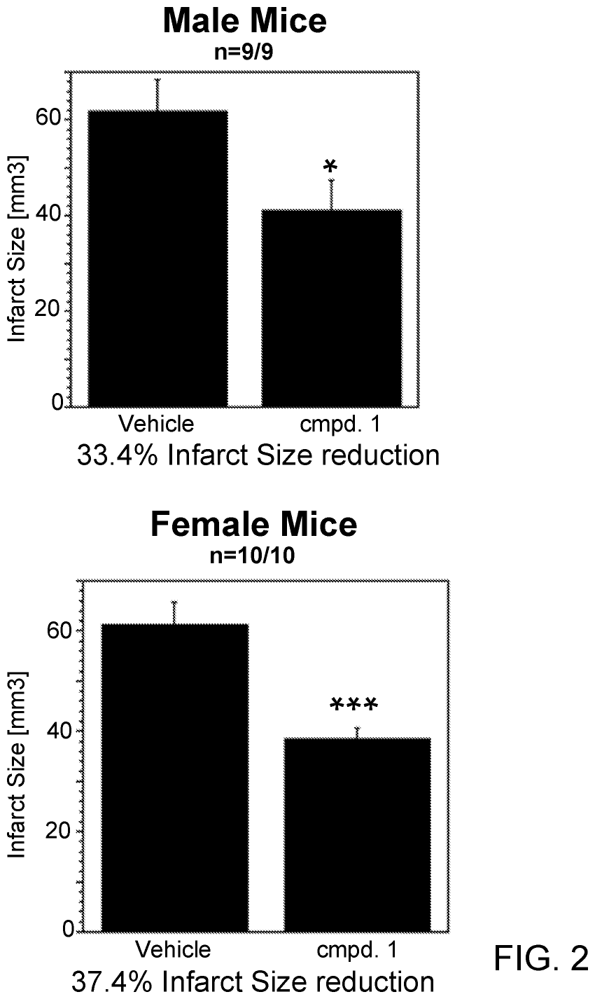

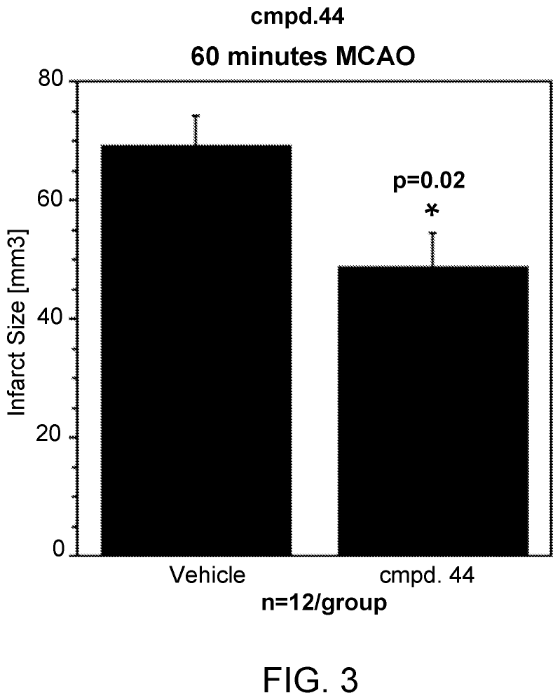

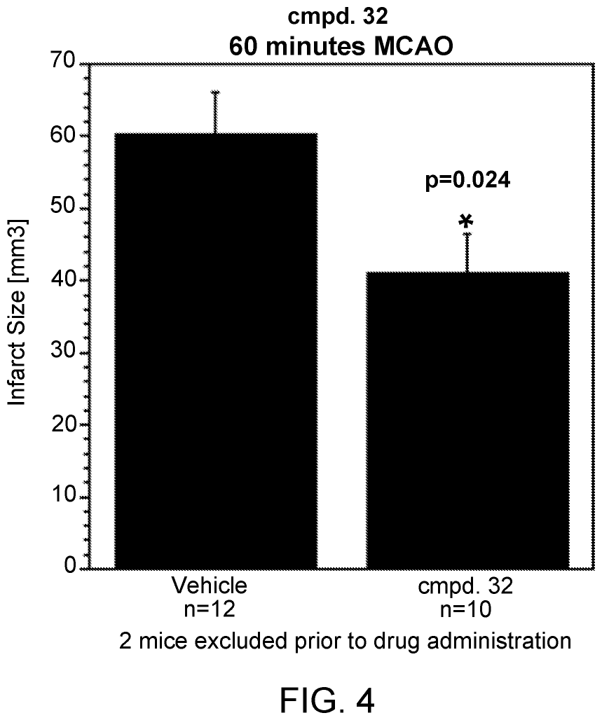

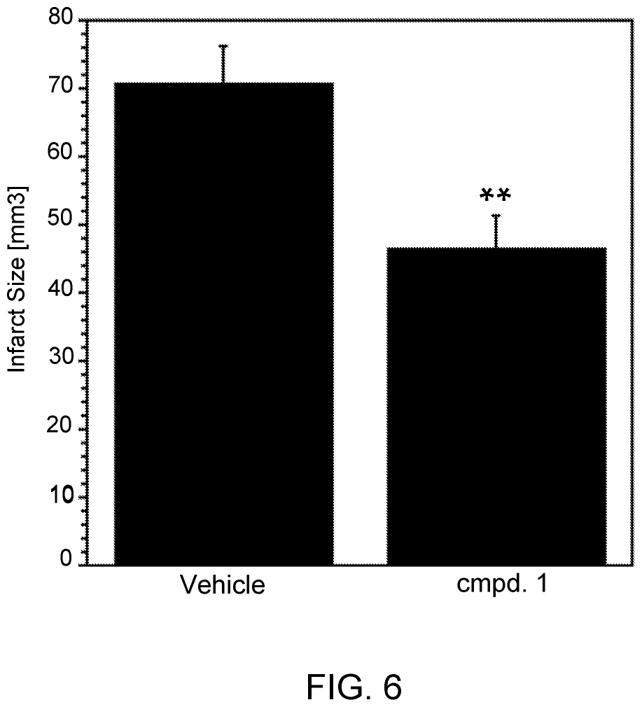

Studies were carried out in experimental stroke model in mice, using the filament model of middle cerebral artery occlusion (MCAO). For intraperitoneal delivery, compounds were dissolved in DMSO. A number of exemplified compounds were able to reduce infarct size when given at a dosage of 40 mg/kg. Results are shown in FIGS. 2-6.

### Example 7—Summary of In Vitro Results for Cmpd. 1

Human 12/15-LOX=0.12 μM (94% max).
Human 12/15-LOX E. coli IC₅₀=0.11 μM (88% max).
Mouse 12/15-LOX E. coli IC₅₀=0.18 μM (82% max).
Human 12/15-LOX HEK EC₅₀=0.92 μM.
Mouse HT22 EC₅₀=5.0 μM.
Rat Primary Neuron EC₅₀=9.5 μM.
12-LOX, 15-LOX2,5-LOX, COX-1 and COX-2>400-fold.

### Example 8—Mouse Stroke Model

40 mg/kg cmpd. 1 were given i.p. at time of reperfusion. Infarct size was measured at 24 hours after 60 minutes MCAO. 33.4% infarct size reduction was observed in male mice (n=9/9), while 37.2% infarct size reduction was observed in female mice (n=10/10).

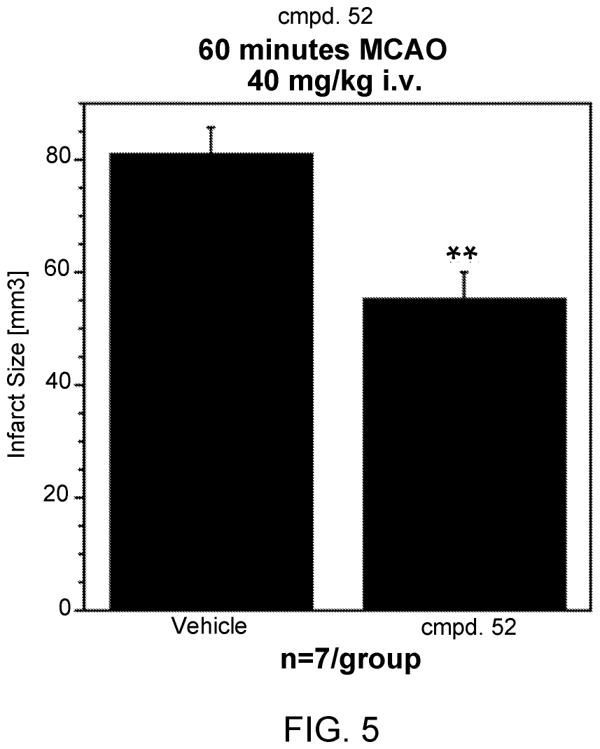

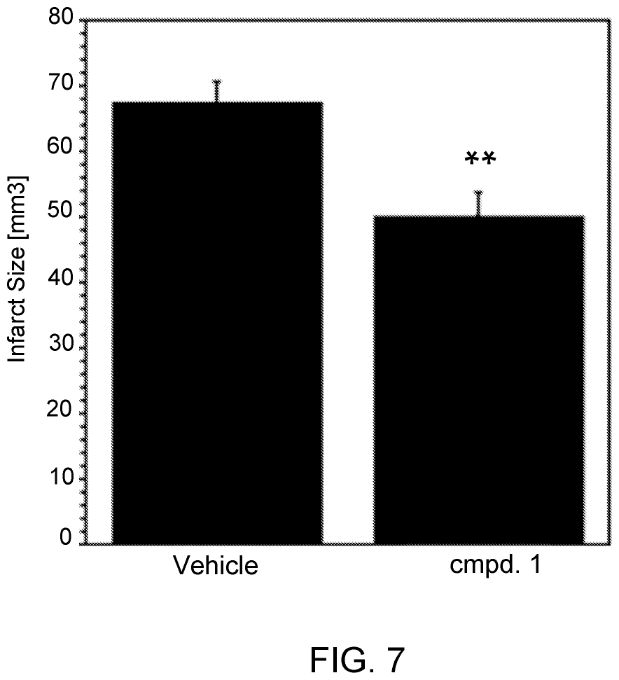

When cmpd. 1 was given i.v. 2 hours post reperfusion, 34.1% infarct size reduction was observed when the compound was administered 40 mg/kg (n=10/11, p<0.01, see FIG. 5B), and 25.8% infarct size reduction was observed when the compound was administered 20 mg/kg (n=17/18, p<0.01, see FIG. 7).

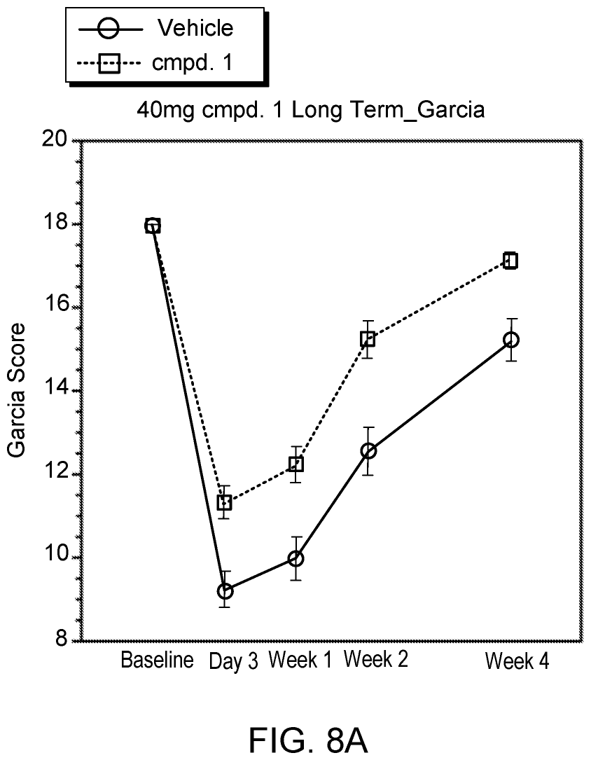

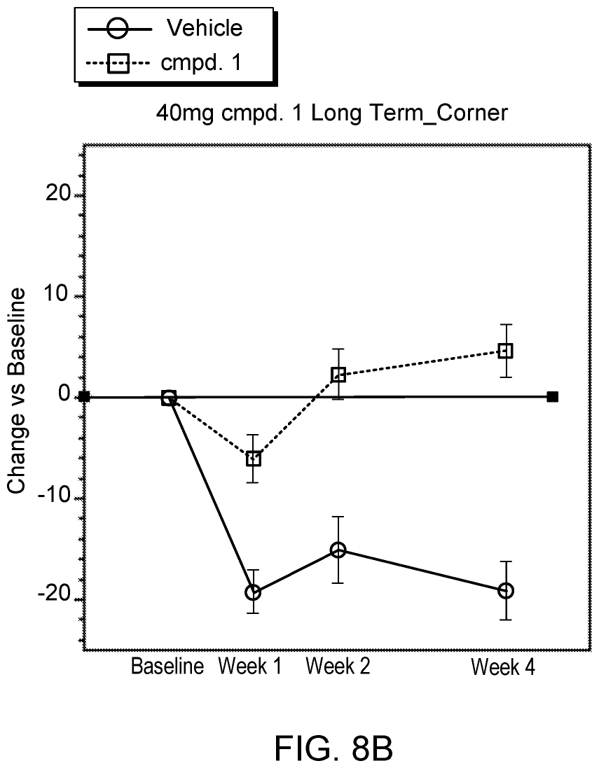

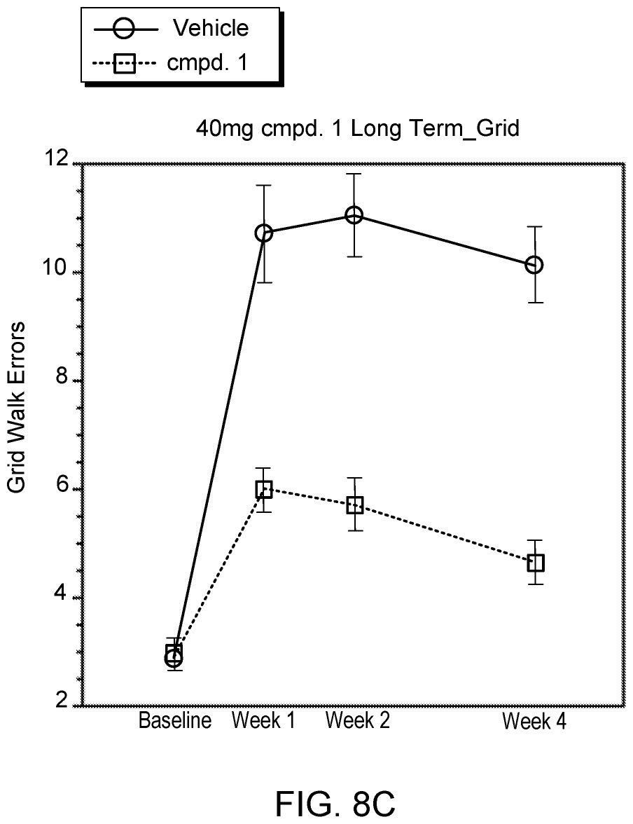

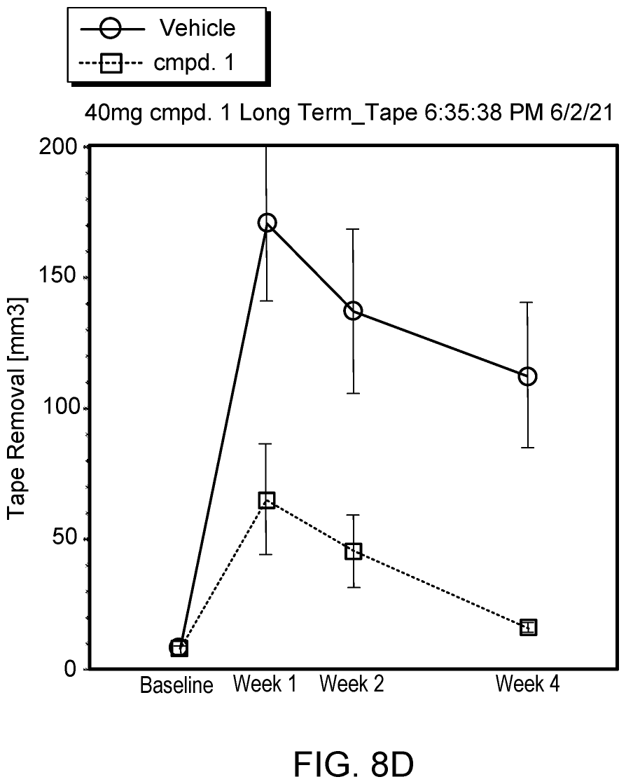

Long-term outcome experiment: 40 mg/kg cmpd. 1 delivered I.V. 2 hours post reperfusion, same as short term, compared to vehicle (10% solutol/90% PEG400). 45 mice operated, and 27 survived, mice were sacrificed on day 30. Behavioral tests included Garcia score (23.8% Day 3, 68.5% Week 4, see FIG. 8A), corner test (68.7% Week 1, 75.4% Week 4, see FIG. 8B), foot fault test (Week 1 61.7%, Week 4 77.0%, see FIG. 8C), Gridwalk test, adhesive removal test (Week 1 65.6%, Week 4 93.3%, see FIG. 8D), and Y maze (% alternation, no significant differences, see FIG. 8E; entry number; no difference between treatment groups, see FIG. 8F).

MCAO (Experimental Middle Cerebral Artery Ischemia and Functional Recovery) experiment: 40 mg/kg of compound delivered intraperitoneally 2 h post reperfusion, 60 minutes MCAO, sacrifice at 24 h, infarct size determined by TTC staining.

| Cmpd. No. | infarct size reduction |
|---|---|
| Cmpd. 44 | 29.6% |
| Cmpd. 32 | 31.8% |
| Cmpd. 52 (given iv in 50% Captisol) | 31.7% |

### Additional Experiments

The effect of the exemplified compounds (which are, e.g., inhibitors of 12/15-LOX as discussed herein) on treating bran injury after subarachnoid hemorrhage can be assessed in a mouse model as described, for example, in Gaberel et al, Stroke, 2019, 50, 520-523, which is incorporated herein by reference in its entirety. Subarachnoid hemorrhage (SAH) is a devastating form of stroke. It may lead to substantial brain injury. The mouse model to study the impact of 12/15-LOX inhibitors on subarachnoid hemorrhage includes C57Bl6 wild-type mice and Alox15 knockout mice. These mice are subjected to SAH using direct blood injection technique. In SAH wild-type mice, half received the 12/15-LOX inhibitor within the instant claims, and half received vehicle. Immunohistochemistry, brain edema, blood-brain barrier leakage and functional outcomes were assessed 1 and 3 days after SAH induction. Generally, SAH leads to increased 12/15-LOX in macrophages of the brain parenchyma, adjacent to the subarachnoid blood. Neuronal cell death after SAH is reduced in the Alox15 knockout mice and in the wild-type model treated with the exemplified compounds (e.g., cmpd. 1 herein), with improved potency as compared to 12/15-LOX inhibitor compound ML351:

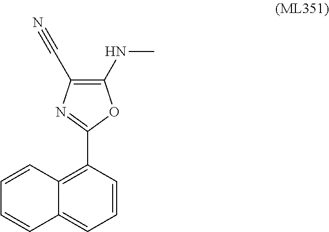 [SMILES: CNc1oc(-c2cccc3ccccc23)nc1C#N]

Also, Alox15 gene knockout and inhibitor treatment in wild-type mice with SAH led to an improved behavioral outcome.

### Animals and Study Design

All animal experiments were performed following protocols approved by the Massachusetts General Hospital Institutional Animal Care and Use Committee in accordance with the National Institutes of Health Guide for the Care and Use of Laboratory Animals. Both the surgeon carrying out the operations, and the investigators evaluating data, were blinded as to treatment groups, except for the functional evaluation of the ALOX15 knockout mice. The reports on this study are compliant with the ARRIVE guidelines. Experiments were performed in 10 week old male ALOX15 knockout mice (n=11, 20-28 g, locally bred; C57B16 genetic background) and age-matched C57B16 wild-type mice (n=80; 20-28 g; Jackson laboratory, Bar Harbor, Maine). Sample sizes were evaluated as follows: based on prior experience, coefficients of variation around 35% between mice are expected. A power analysis using an alpha=0.05, beta-0.8 suggests that for effect sizes of about 40-50% (which we can typically expect for a successful neuroprotectant), the minimum n required for functional tests comparisons are 16, and for brain water content and Blood brain barrier permeability tests it is approximately 8. Here, it was increased slightly to 20 animals per group, except for ALOX15 knockout mice, due to their low availability. For all the surgical procedure, mice were anesthetized with isoflurane (2%) in 70%/30% NO₂/O₂. Body temperature was maintained at 37° C. with a rectal temperature probe and homeothermic heating pad.

### Subarachnoid Hemorrhage Induction

Subarachnoid hemorrhage (SAH) was induced by injection of fresh arterial blood into the prechiasmatic cistern. The mouse was placed within a stereotactic frame. A hole was drilled in the skull 4.5 mm anterior to the bregma, avoiding the superior sagittal sinus. A 30-gauge needle was advanced at a 40° angle for 5 mm until the skull base was touched. It was withdrawn from 0.5-1 mm, so that the tip was in the prechiasmatic cistern. Arterial blood was withdrawn from the left ventricle of another anesthetized mouse using a 25 gauge needle. 100 μL of blood were manually injected through the 30 gauge needle in 15 seconds. For sham animals, the needle was inserted within the prechiasmatic cistern, and no injection was made. For all the animals the needle was then removed and the wound closed. The animals were then allowed to recover. All the mice were included in the final analyses

### Drug Administration

Five minutes after SAH induction, mice received a single intraperitoneal injection using a 30-gauge needle (120 μL) of DMSO (Vehicle) or test compound (e.g., ML-351) dissolved in DMSO (test compound such as ML-351 at a concentration of 12.5 mg/ml, giving a dose of 50 mg/kg). The dosage was chosen according to the studies of focal ischemia in mice. Animals were then allowed to recover. The injections were performed by an investigator blinded as to treatment group, and a predefined randomization list was used to assign mice to a treatment group.

### Behavioral Experiments

Except the mice used for IHC 24 h after SAH induction, all mice were assessed at 72 hours using a 4-point Neuroscore scale, a 18 point Neuroscore scale, and by evaluating spontaneous activity. Four point Neuroscore scale was defined as: 0=no apparent deficit; 1=slight deficit; 2-circling; 3-heavy circling or no movement at all; or 4=death. The 18 point Neuroscore scale evaluated 6 parameters, each noted from 0 to 3.5. Spontaneous locomotor activity was evaluated by counting the horizontal movements. An open field Plexiglas chamber was used, in which 4 lines were drawn to delineate 9 fields. Mice were placed in the chamber for 5 minutes to acclimate. Then a video was recorded for 5 minutes. The videos were then analyzed by an investigator blinded as to treatment group. The investigator manually counted the number of fields explored by the mice during 5 minutes.

### OTHER EMBODIMENTS

It is to be understood that while the present application has been described in conjunction with the detailed description thereof, the foregoing description is intended to illustrate and not limit the scope of the present application, which is defined by the scope of the appended claims. Other aspects, advantages, and modifications are within the scope of the following claims.

1. A compound of Formula (I): 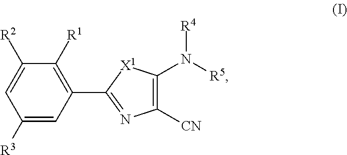
or a pharmaceutically acceptable salt thereof, wherein:
X¹ is selected from O and S;
R¹, R², and R³ are each independently selected from halo, CN, C₁₋₃ alkyl, C₁₋₃ haloalkyl, C₁₋₃ alkoxy, and C₁₋₃ haloalkoxy;
R⁴ is selected from H, C₁₋₃ alkyl, and HO—C₁₋₃ alkylene;
R⁵ is selected from C₁₋₆ alkyl, C₂₋₆ alkenyl, C₂₋₆ alkynyl, C(O)OR^{a1}, C(O) N(R^{a1})₂, P(═O)(OR^{a1})₂, and C(O)R^{b1}; wherein said C₁₋₆ alkyl, C₂₋₆ alkenyl, and C₂₋₆ alkynyl are each optionally substituted with a substituent selected from OR^{a1} and OP(═O)(OR^{a1})₂;
each R^{a1} is independently selected from H, C₁₋₆ alkyl, C₆₋₁₀ aryl, C₁₋₆ alkyl-C₆₋₁₀ aryl, and C₁₋₆ alkyl-C₆₋₁₀ aryl-C₁₋₆ alkyl, wherein said C₁₋₆ alkyl, C₆₋₁₀ aryl, C₁₋₆ alkyl-C₆₋₁₀ aryl, and C₁₋₆ alkyl-C₆₋₁₀ aryl-C₁₋₆ alkyl are each optionally substituted with a substituent selected from amino, C₁₋₆ alkylamino, (C₁₋₆ haloalkyl)amino, di(C₁₋₆ alkyl)amino, (C₁₋₆ alkyl) (C₁₋₆ haloalkyl)amino, (C₆₋₁₀ aryl)amino, (C₆₋₁₀ aryl) (C₁₋₆ alkyl)amino, (5-6-membered heteroaryl)amino, (5-6-membered heteroaryl) (C₁₋₆ alkyl)amino, C₆₋₁₀ aryl, 4-6 membered heterocycloalkyl, 5-6-membered heteroaryl, and OR^{a2}, wherein said C₆₋₁₀ aryl, 4-6 membered heterocycloalkyl, and 5-6-membered heteroaryl are each optionally substituted with 1, 2, or 3 substituents independently selected from amino, C₁₋₆ alkylamino, di(C₁₋₆ alkyl)amino, carboxy, and halo;
each R^{a2} is independently selected from H, C₁₋₃ alkyl, C₁₋₃ haloalkyl, C₁₋₃ alkoxy-C₁₋₃ alkyl, 4-7 membered heterocycloalkyl-C₁₋₃ alkyl, 5-6-membered heteroaryloxy-C₁₋₃ alkyl, C₆₋₁₀ aryl, and 5-6-membered heteroaryl, wherein said C₆₋₁₀ aryl and 5-6-membered heteroaryl are each optionally substituted with 1, 2, or 3 substituents independently selected from halo, C₁₋₃ alkoxy, C₁₋₃ haloalkoxy, C₁₋₃ alkyl, and C₁₋₃ haloalkyl; and
R^{b1} is C₁₋₆ alkyl, optionally substituted with a substituent selected from amino, C₁₋₆ alkylamino, di(C₁₋₆ alkyl)amino, and 4-7 membered heterocycloalkyl ring comprising at least one N atom.

2. The compound of claim 1, wherein the compound has formula: 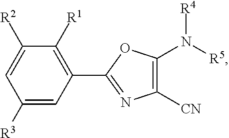
or a pharmaceutically acceptable salt thereof.

3. The compound of claim 1, wherein the compound has formula: 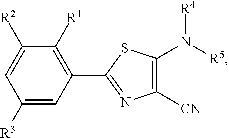
or a pharmaceutically acceptable salt thereof.

4. The compound of any one of claim 1, wherein R¹, R², and R³ are each halo.

5. The compound of any one of claim 1, wherein:
R² and R³ are each halo; and
R¹ is selected from C₁₋₃ alkyl, C₁₋₃ alkoxy, and C₁₋₃ haloalkoxy.

6. The compound of any one of claim 1, wherein:
R¹ and R³ are each halo; and
R² is selected from C₁₋₃ alkyl, C₁₋₃ alkoxy, and C₁₋₃ haloalkyl.

7. The compound of any one of claim 1, wherein:
R¹ and R² are each halo; and
R³ is selected from C₁₋₃ alkyl, C₁₋₃ haloalkyl, and CN.

8. The compound of any one of claim 1, wherein the compound has formula: 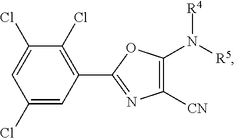
or a pharmaceutically acceptable salt thereof.

9. The compound of any one of claim 1, wherein R⁵ is C₁₋₃ alkyl.

10. The compound of any one of claim 1 wherein R⁵ is C₂₋₆ alkenyl, optionally substituted with OR^{a1}.

11. The compound of any one of claim 1, wherein R⁵ is C₂₋₆ alkynyl, optionally substituted with OR^{a1}.

12. The compound of any one of claim 1, wherein R⁵ is C₁₋₆ alkyl substituted with OR^{a1}.

13. The compound of any one of claim 1, wherein R⁵ is C₁₋₆ alkyl substituted with OP(═O)(OR^{a1})₂.

14. The compound of any one of claim 1, wherein R^{a1} is H.

15. The compound of any one of claim 1, wherein R^{a1} is C₁₋₆ alkyl, optionally substituted with C₆₋₁₀ aryl or OR^{a2}.

16. The compound of any one of claim 1, wherein R⁴ is H.

17. The compound of any one of claim 1, wherein R⁵ is C(O)OR^{a1}.

18. The compound of claim 17, wherein R^{a1} is C₁₋₆ alkyl, optionally substituted with a substituent selected from amino, C₁₋₆ alkylamino, and di(C₁₋₆ alkyl)amino.

19. The compound of any one of claim 1, wherein R⁵ is C(O)R^{b1}.

20. The compound of any one of claim 1, wherein:
R⁴ is selected from H, C₁₋₃ alkyl, and HO—C₁₋₃ alkylene; and
R⁵ is selected from C₁₋₆ alkyl, C₂₋₆ alkenyl, and C₂₋₆ alkynyl, each of which is optionally substituted with a substituent selected from OR^{a1} and OP(═O)(OR^{a1})₂; and
each R^{a1} is independently selected from H and C₁₋₆ alkyl, wherein said C₁₋₆ alkyl is optionally substituted with a substituent selected from C₆₋₁₀ aryl and OR^{a2}.

21. The compound of any one of claim 1, wherein:
R⁴ is selected from C(O)OR^{a1} and C(O)R^{b1};
R⁵ is C₁₋₃ alkyl; and
R^{a1} is C₁₋₆ alkyl, optionally substituted with a substituent selected from amino, C₁₋₆ alkylamino, and di(C₁₋₆ alkyl)amino.

22. The compound of claim 1, wherein the compound of Formula (I) is selected from any one of the following compounds: 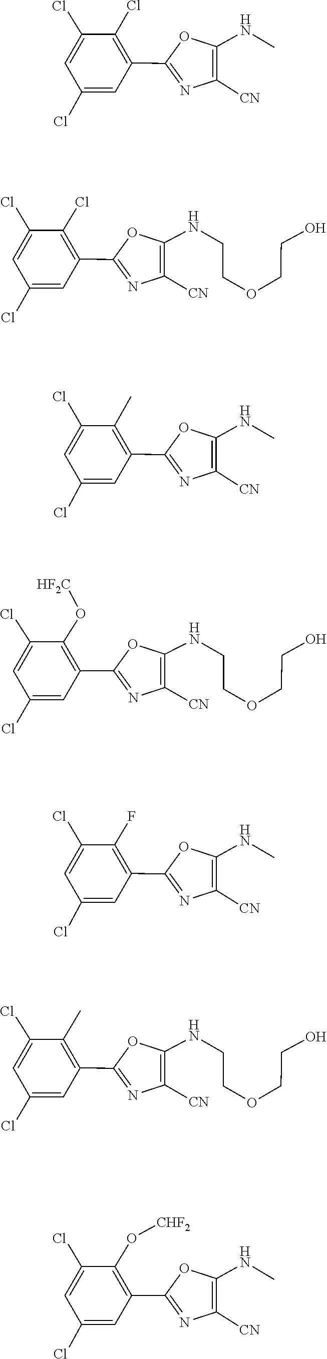 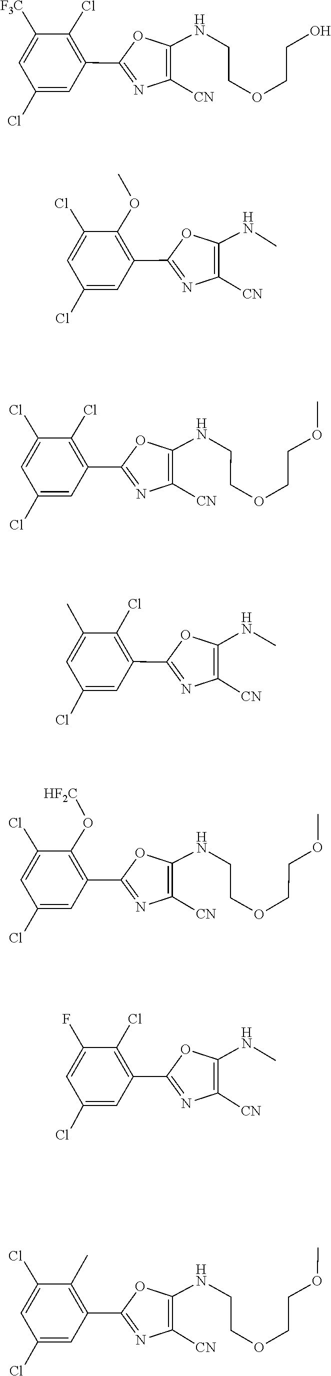 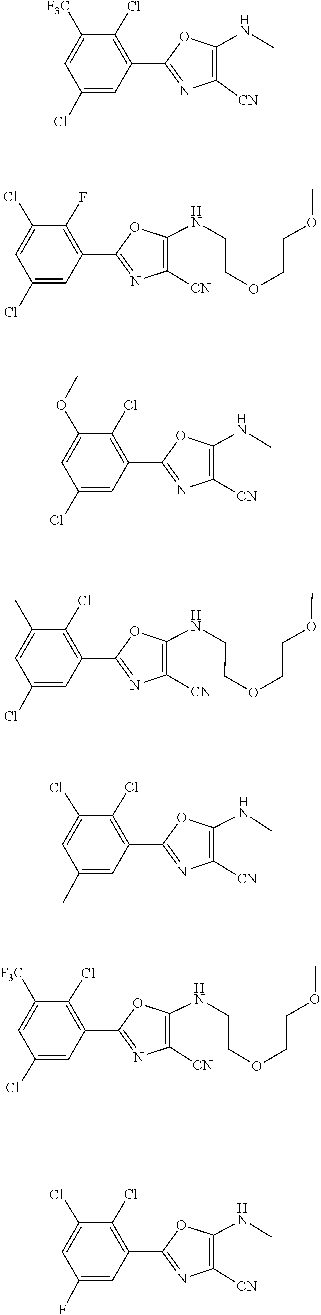 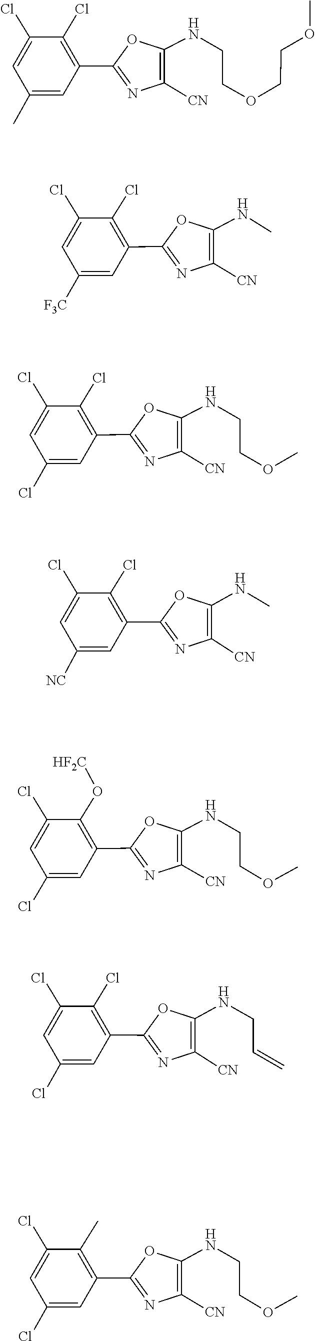 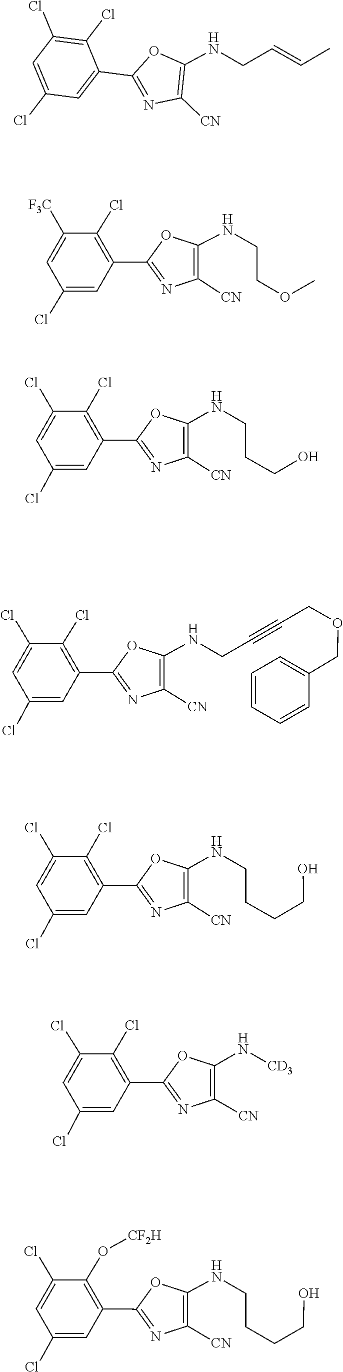 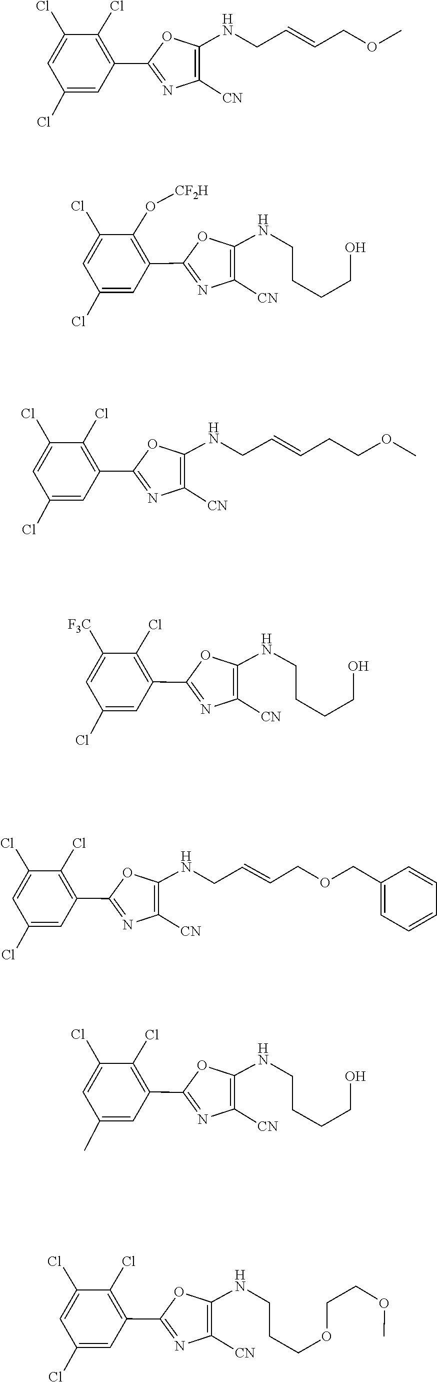 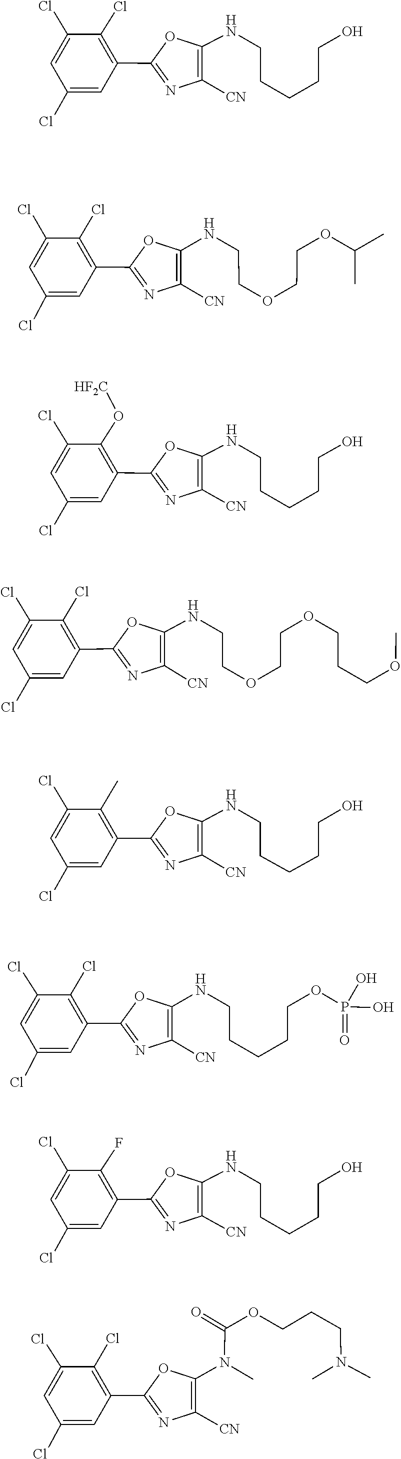 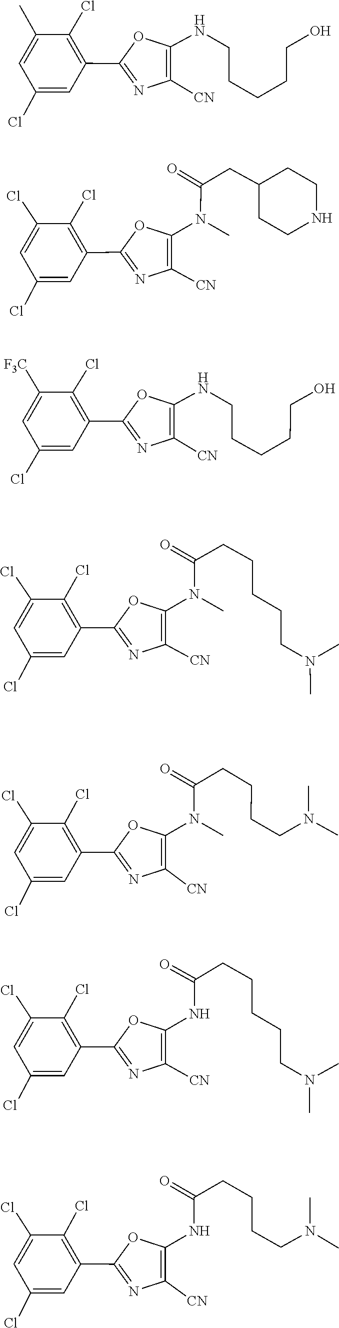 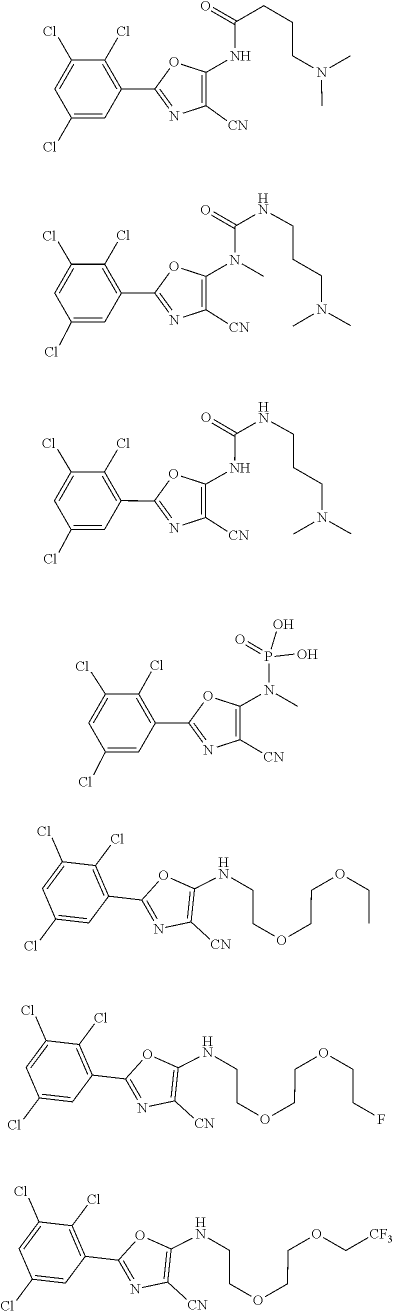 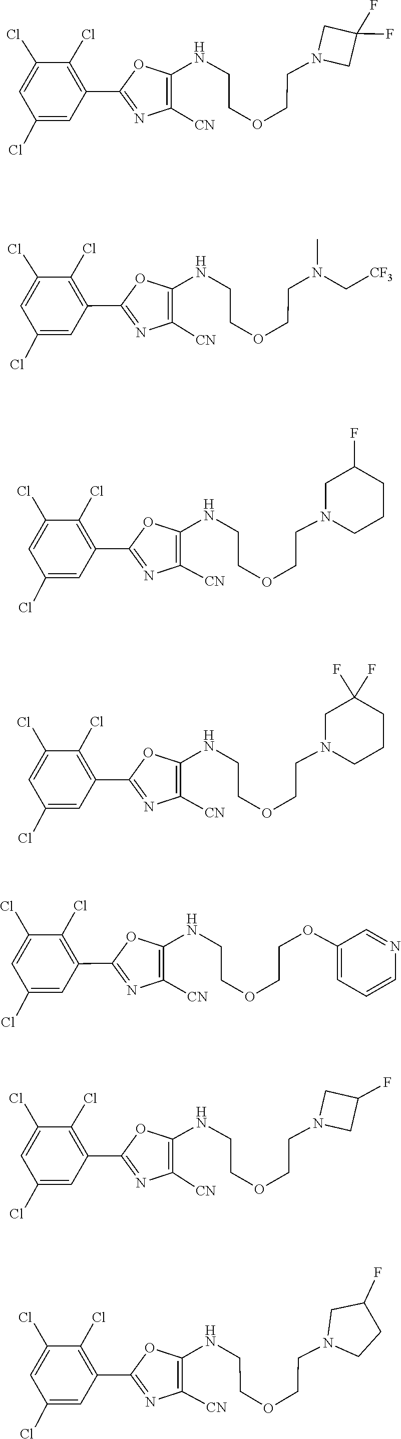 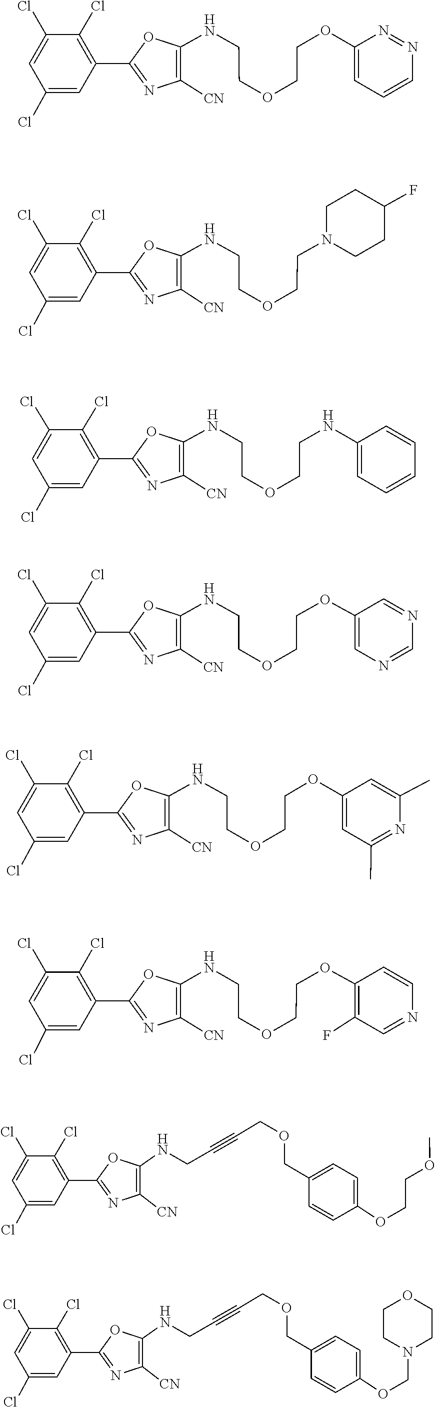 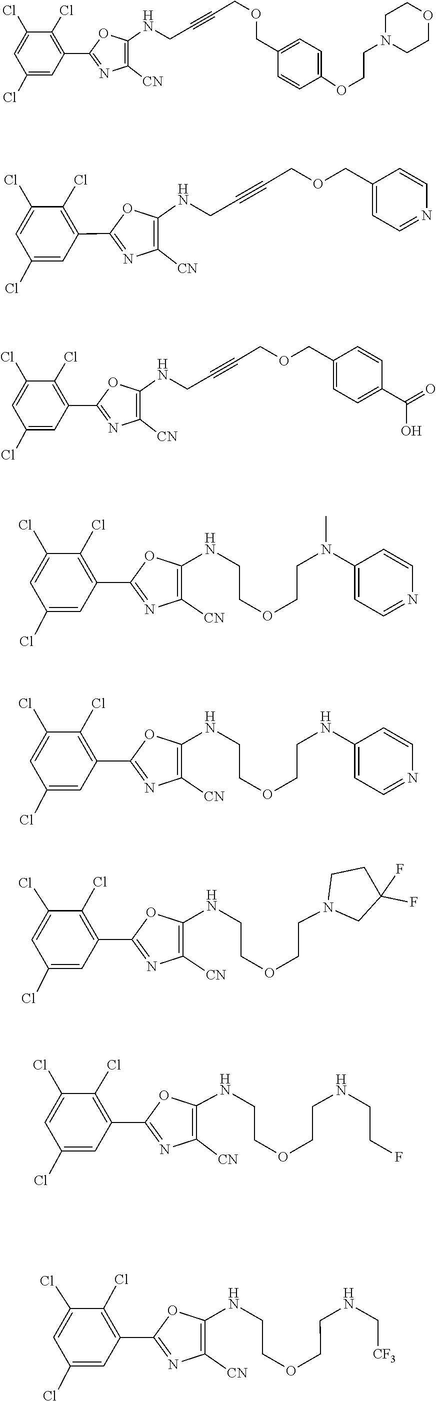 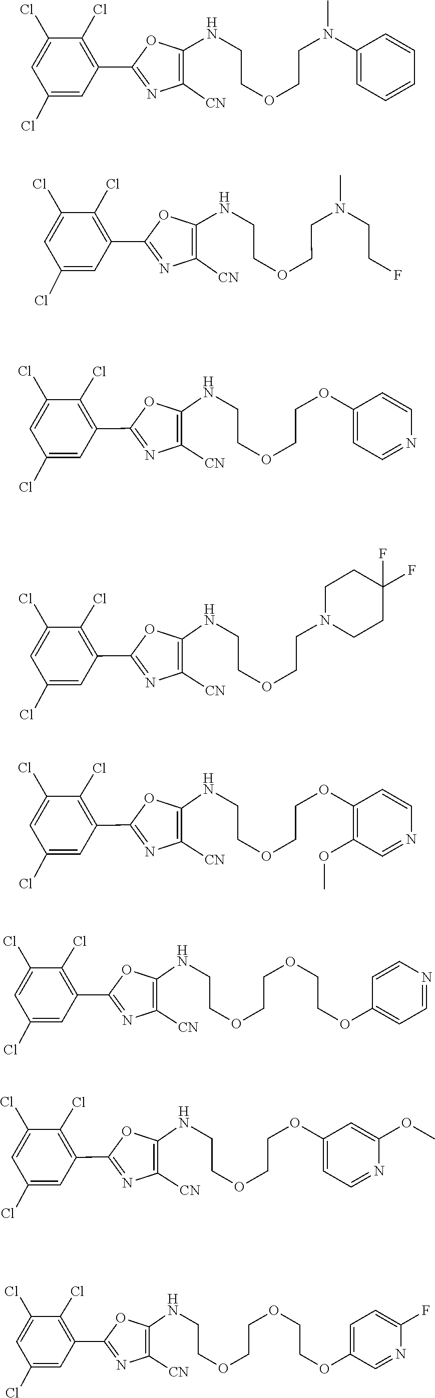 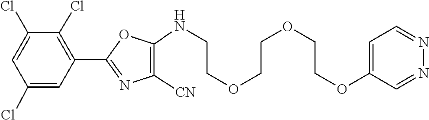 [SMILES: N#Cc1nc(-c2cc(Cl)cc(Cl)c2Cl)oc1NCCOCCOCCOc1ccnnc1]
or a pharmaceutically acceptable salt thereof.

23. A pharmaceutical composition comprising a compound of any one of claim 1, or a pharmaceutically acceptable salt thereof, and a pharmaceutically acceptable carrier.

24. A method of treating or preventing a disease or disorder in which 12/15-lipoxygenase (LOX) is implicated in the pathology, the method comprising administering to a subject in need thereof a therapeutically effective amount of a compound of any one of claim 1, or a pharmaceutically acceptable salt thereof.

25. The method of claim 24, wherein the disease or disorder is selected from: stroke, diabetes, obesity, asthma, glomerulonephritis, osteoporosis, periventricular leukomalacia, cardiac arrest with resuscitation, atherosclerosis, neurodegenerative or neuroinflammatory disorders (e.g., Parkinson's disease, Alzheimer's disease, or dementia), cancer, brain injury, a disease involving hypoxia or anoxia, myocardial infarction, cardiovascular disease, heart failure (e.g., chronic or congestive heart failure), ischemia (e.g., cerebral ischemia, retinal ischemia, myocardial ischemia, or post-surgical cognitive dysfunction), inflammatory disease (e.g., arterial inflammation, inflammatory bowel disease, Crohn's disease, renal disease, asthma, allergic rhinitis, gout, cardiopulmonary inflammation, rheumatoid arthritis, osteoarthritis, muscle fatigue, acne, dermatitis, or psoriasis), chronic bronchitis, mucus hypersecretion, chronic obstructive pulmonary disease (COPD), pulmonary fibrosis (including fibrosis caused by chemotherapy), idiopathic pulmonary fibrosis, cystic fibrosis, adult respiratory distress syndrome, CNS disorders, psychiatric disorders (e.g., anxiety or depression), peripheral neuropathy (e.g., spinal cord injury, head injury, or surgical trauma), allograft tissue or organ transplant rejection, autoimmune disorder (e.g., eczema), and disorders involving bone loss or bone formation.
